(19) United States
(12) Patent Application Publication
(10) Pub. No.: US 2026/0354373 A1
(43) Pub. Date: Nov. 12, 2026

(54) PERFUSION AND METABOLIC RESTORATION OF ORGANS

(71) Applicant: Bexorg, Inc., New Haven, CT (US)

(72) Inventors: Zvonimir Vrselja, New Haven, CT (US); Bradley Parry, Branford, CT (US); Brian Hinger, East Haven, CT (US)

(21) Appl. No.: 19/663,994

(22) Filed: Apr. 30, 2026

Related U.S. Application Data
(60) Provisional application No. 63/802,411, filed on May 8, 2025.
(60) Provisional application No. 63/995,295, filed on Mar. 3, 2026.

(51) Int. Cl. A01N 1/143; A01N 1/144; A01N 1/146; A01N 1/165; G06N 20/00

(52) U.S. Cl. CPC A01N 1/143; A01N 1/144; A01N 1/146; A01N 1/165; G06N 20/00

(57) ABSTRACT

An ex-vivo brain perfusion system for perfusion of a brain includes a reservoir configured to receive perfusate, a housing configured to contain the brain, an arterial circuit configured to fluidly couple the reservoir to the brain; a venous circuit fluidly coupling the housing to the reservoir; at least one sensor configured to generate data indicating one or more characteristics of the perfusate, one or more processors configured to execute a machine learning model to process the data generated by the at least one sensor and determine one or more chemical components to be added to the perfusate to extend the length of perfusion, and a syringe pump manifold fluidly coupled to the reservoir and configured to automatically introduce the one or more chemical components determined by the machine learning model.

### CROSS-REFERENCE TO RELATED APPLICATIONS

This application claims priority to U.S. Application Ser. No. 63/802,411, filed on May 8, 2025 and U.S. Application Ser. No. 63/995,295, filed on Mar. 3, 2026. The disclosure of the prior application is considered part of the disclosure of this application, and is incorporated in its entirety into this application.

### TECHNICAL FIELD

This specification relates to methods, systems, and compositions for perfusion and metabolic restoration of organs, in particular mammalian brains.

### BACKGROUND

Perfusion in mammalian brains refers to the process of delivering perfusate (e.g., blood or an artificial perfusate) to the brain tissue to supply inputs, such as oxygen, glucose, and nutrients, while removing outputs, such as carbon dioxide and metabolic wastes. The process of perfusion is facilitated by a dense network of blood vessels, including arteries, capillaries, and veins, which collectively carry the cerebral blood flow.

### SUMMARY

In one aspect, a method performed by one or more computers, the method includes: automatically controlling an ex-vivo brain perfusion device using a controller machine learning model, including, at each of a plurality of time steps: receiving a current observation characterizing a current state of a brain being perfused by the ex-vivo brain perfusion device; processing the current observation using the controller machine learning model to generate an output that defines one or more actions to be performed by the ex-vivo brain perfusion device; and performing the one or more actions using the ex-vivo brain perfusion device.

Implementations can include one or more of the following features in any combination

In some implementations, the method further includes determining, at each of the plurality of time steps, a reward for the time step based at least in part on a level of homeostasis of the brain; and training the controller machine learning model on the rewards for the plurality of time steps using a reinforcement learning training technique.

In some implementations, training the controller machine learning model on the rewards for the plurality of time steps using the reinforcement learning training technique includes training the controller machine learning model to increase a cumulative measure of rewards received by controlling the ex-vivo brain perfusion device using the controller machine learning model.

In some implementations, the reinforcement learning training technique includes an actor-critic reinforcement learning technique or a policy gradient reinforcement learning technique.

In some implementations, for each of the plurality of time steps, determining the reward for the time step includes determining the reward for the time step based at least in part on a discrepancy between: (i) a current metabolic state of the brain at the time step, and (ii) a target metabolic state of the brain.

In some implementations, determining the reward for the time step based at least in part on the discrepancy between: (i) the current metabolic state of the brain at the time step, and (ii) the target metabolic state of the brain, includes: determining the current metabolic state of the brain at the time step based on sensor measurements generated by Raman spectroscopy of perfusate that is being used to perfuse the brain.

In some implementations, for each of the plurality of time steps, determining the reward for the time step includes determining the reward for the time step based at least in part on a discrepancy between: (i) a current mechanical state of the perfusate at the time step, and (ii) a target mechanical state of the perfusate.

In some implementations, determining the reward for the time step based at least in part on the discrepancy between: (i) the current mechanical state of the perfusate at the time step, and (ii) the target mechanical state of the perfusate, includes: determining the current mechanical state of the perfusate at the time step based on one or more of: current apparent viscosity of the perfusate; current convective-diffusive flux of the perfusate; or a current arterial inflow waveform of the perfusate.

In some implementations, the controller machine learning model includes one or more of: a neural network model, or a linear model, or a Gaussian process model.

In some implementations, performing the one or more actions using the ex-vivo brain perfusion device includes: modifying one or more chemical parameters of a perfusate being used to perfuse the brain.

In some implementations, modifying one or more chemical parameters of a perfusate being used to perfuse the brain includes modifying one or more of: a viscosity of the perfusate; a pH of the perfusate; or a chemical composition of the perfusate.

In some implementations, performing the one or more actions using the ex-vivo brain perfusion device includes: modifying one or more operational parameters of the ex-vivo brain perfusion device.

In some implementations, modifying one or more operational parameters of the ex-vivo brain perfusion device causes modifications to one or more of: a flow waveform of a perfusate being used to perfuse the brain; a degassing rate of the perfusate being used to perfuse the brain; or a pressure applied to the perfusate being used to perfuse the brain.

In some implementations, receiving the current observation characterizing the current state of the brain being perfused by the ex-vivo brain perfusion device includes obtaining current sensor measurements generated by one or more sensors of the ex-vivo brain perfusion device; and generating the current observation based on the current sensor measurements.

In some implementations, the current observation characterizes at least a current mechanical state of perfusate being used to perfuse the brain.

In some implementations, the current observation characterizes the current mechanical state of the perfusate by one or more of: current apparent viscosity of the perfusate; a current arterial inflow waveform of the perfusate; a current flow resistance of the perfusate; and a current convective-diffusive flux of the perfusate.

In some implementations, the current observation characterizes at least a current metabolic state of the brain.

In some implementations, the current observation characterizes the current metabolic state of the brain by one or more of: concentrations of one or more metabolites in perfusate being used to perfuse the brain; oxygen partial pressure in the perfusate; carbon dioxide partial pressure in the perfusate; cerebral metabolic rate of oxygen; cerebral metabolic rate of glucose; oxygen-glucose indices; or lactate-glucose indices.

In some implementations, the one or more metabolites comprise one or more of: glucose, lactate, pyruvate, or glutamate.

In some implementations, processing the current observation using the controller machine learning model to generate the output that defines one or more actions to be performed by the ex-vivo brain perfusion device includes: processing the current observation using the controller machine learning model to generate a score distribution over a set of possible actions; and selecting one or more actions to be performed by the ex-vivo brain perfusion device in accordance with the score distribution.

In some implementations, selecting one or more actions to be performed by the ex-vivo brain perfusion device in accordance with the score distribution includes: selecting one or more actions having highest scores under the score distribution.

In a further aspect, a system includes: one or more computers; and one or more storage devices communicatively coupled to the one or more computers, wherein the one or more storage devices store instructions that, when executed by the one or more computers, cause the one or more computers to perform operations of the methods described herein.

In a further aspect, one or more non-transitory computer storage media storing instructions that when executed by one or more computers cause the one or more computers to perform operations of the methods described herein.

In a further aspect, an ex-vivo brain perfusion system for perfusion of a brain includes a reservoir configured to receive perfusate; a housing configured to contain the brain; a housing stand configured to automatically adjust a position of the brain housing; an arterial circuit configured to fluidly couple the reservoir to the brain; and a venous circuit fluidly coupling the housing to the reservoir.

Implementations can include one or more of the following features in any combination.

In some implementations, the housing stand includes a base; a plurality of legs extending from the base; and at least one linear actuator, wherein the at least one linear actuator is configured to extend and retract to automatically adjust the position of the brain housing.

In some implementations, the at least one linear actuator is configured to extend and retract at predefined intervals throughout the perfusion of the brain.

In some implementations, the system includes at least one sensor configured to generate data indicate tissue viability of the brain; and the at least one linear actuator is configured to extend and retract based on data received from the at least one sensor.

In some implementations, the at least one sensor includes at least one of a camera, a Raman spectrometer, a colorimeter, a fluorimeter, or a photometer.

In a further aspect, an ex-vivo brain perfusion system for perfusion of a brain includes a reservoir configured to receive perfusate; a housing configured to contain the brain; an arterial circuit configured to fluidly couple the reservoir to the brain; a venous circuit fluidly coupling the housing to the reservoir; at least one sensor configured to generate data indicating one or more characteristics of the perfusate; one or more processors configured to execute a machine learning model to process the data generated by the at least one sensor and determine one or more chemical components to be added to the perfusate to extend the length of perfusion; and a syringe pump manifold fluidly coupled to the reservoir and configured to automatically introduce the one or more chemical components determined by the machine learning model.

Implementations can include one or more of the following features in any combination.

In some implementations, the at least one sensor includes at least one of a Raman spectrometer, a colorimeter, a fluorimeter, or a photometer, a conductivity sensor, a pH sensor, a temperature sensor, or a viscosity sensor.

In some implementations, the syringe pump manifold is configured to automatically introduce the one or more chemical components based on one or more command signals generated by the machine learning model.

In some implementations, the system includes a filtration system; the machine learning model is configured to determine one or more chemical components to be removed to the perfusate to extend the length of perfusion; and the filtration system is configured to be operated to remove the one or more chemical components from the perfusate in the reservoir.

In some implementations, the filtration system includes a dialyzer manifold including a plurality of dialyzers; and the dialyzer manifold is configured to automatically divert flow of perfusate from a first dialyzer of the plurality of dialyzers to a second dialyzer of the plurality of dialyzers based on a sensor of the filtration system generating data indicating that the first dialyzer is clogged or malfunctioning.

In some implementations, the filtration system includes a dialysis solution receptacle configured to receive fluid flowing out of the dialyzer manifold; and a pump configured to pump fluid from the dialysis solution receptacle to the reservoir; the data generated by the at least one sensor indicates a ratio of small molecules to large molecules present in the perfusate in the reservoir; and the pump is controlled to pump fluid from the dialysis solution receptacle to the reservoir in response to a determination that the ratio of small molecules to large molecules present in the perfusate in the reservoir is below a first threshold.

In some implementations, the filtration system is configured to flow fluid from the reservoir through the dialyzer manifold in response to a determination that the ratio of small molecules to large molecules present in the perfusate in the reservoir is above a second threshold.

In some implementations, the filtration system includes a filter manifold including a plurality of mechanical filters; and the filter manifold is configured to automatically divert flow of perfusate from a first mechanical filter of the plurality of mechanical filters to a second mechanical filter of the plurality of mechanical filter based on a sensor of the filtration system generating data indicating that the first mechanical filter is clogged or malfunctioning.

In some implementations, the sensor of the filtration system includes at least one of a pressure sensor or a flow sensor.

In some implementations, the data generated by the at least one sensor is configured to be processed by the machine learning model to determine viability of tissue of the brain; the machine learning model is configured to determine at least one chemical component to be added to the perfusate to improve viability of the tissue of the brain; and the syringe pump manifold is configured to introduce the at least one chemical component identified by the machine learning model

In some implementations, the at least one sensor is a camera configured to take image data depicting the brain; and the machine learning model is configured to perform machine learning based image processing on the image data to determine the viability of the tissue of the brain in real time.

In some implementations, the system includes an impeller positioned within the reservoir and configured to mix the perfusate contained in the reservoir.

In some implementations, the one or more chemical components includes at least one of an oxygen carrier molecule, a pH buffer, a metabolite, or a hormone.

In some implementations, the metabolite includes at least one of glucose, lactate, pyruvate, or glutamate.

In some implementations, the perfusion system includes a supplemental reservoir configured to contain fresh perfusate; and a supplement pump configured to pump fresh perfusate from the supplemental reservoir to the reservoir.

In some implementations, the supplement pump is configured to pump fresh perfusate from the supplemental reservoir to the reservoir at predetermined intervals throughout perfusion of the brain.

In some implementations, the perfusion system includes a viscosity sensor positioned downstream of the reservoir; and the supplement pump is configured to pump fresh perfusate from the supplemental reservoir to the reservoir based on data generated by a viscosity sensor indicating that a viscosity of the perfusate in the reservoir is above a threshold viscosity.

In some implementations, the system includes a microdialysis system fluidly coupled to the housing and configured to perform dialysis on interstitial fluid of the brain.

In some implementations, the microdialysis system includes a membrane configured to be inserted into the brain or positioned on a surface of the brain; a microdialysis syringe pump configured to pump a buffer solution to the membrane; and a collection receptacle configured to receive filtered interstitial fluid flowing from the brain and through the membrane.

In some implementations, the system includes a housing pressurization system including: an air supply; a fluid line fluidly coupling the air supply to the housing; and a pressure regulator configured to control the pressure of air flowing from the air supply into the housing.

In some implementations, the housing pressurization system includes a pressure sensor configured to measure pressure inside the housing; and the pressure regulator is an electronic pressure regulator configured to be controlled based on one or more signals generated by the pressure sensor.

In some implementations, the housing pressurization system includes a filter configured to filter the air flowing from the air supply into the housing.

Implementations can include one or more of the following advantages. The perfusion systems and methods described herein enable restoration and preservation of a human brain, including normal cellular activity of the brain, for up to 2 weeks or longer post-mortem. As a result, cellular and metabolic health of the brain can be preserved in the brain.

In addition, the systems and methods described herein can be used to automatically analyze the chemical composition and rheological properties of the perfusate flowing through the perfusion system in real-time. Further, the systems and methods described herein systems and methods described herein can monitor and predict the tissue viability of a human brain being perfused by the system in real-time without samples of the brain tissue to be removed from the brain. Based on processing the sensor data related to the chemical composition and rheological properties of the perfusate and the health of the brain tissue (e.g., by a machine learning model), the perfusion system can automatically adjust the chemical composition of the perfusate and/or the operation of one or more of the mechanical components of the perfusion system to improve the health of the brain. As a result, a brain can be perfused using the systems and methods described herein for a prolonged period (e.g., two weeks or longer) with minimal operator intervention.

The perfusion systems and methods described herein can also be used to maintain the brain at normothermic conditions and extend the length of tissue viability during perfusion. For example, the brain housing of the perfusion systems described herein can be pressurized and/or dynamically repositioned to reduce gravitational and contact-induced mechanical stress on brain tissue and improve the brain tissue viability.

The perfusion systems and methods described herein can automatically identify malfunctioning system components and/or sub-systems and can automatically shut down or otherwise isolate malfunctioning components and/or sub-systems. As a result, the perfusion systems and methods described herein can maintain process stability and protect brain viability with minimal operator intervention during a sub-system or component malfunction. In addition, the perfusion systems and methods described herein can perform automated quality control checks and calibration of the perfusion system, allowing a perfusion system to be prepared for perfusion with minimal operator intervention.

The perfusion systems described herein can be provided with a fully modular hardware architecture formed of plug-and-play components. As a result, assembling, reconfiguration, and troubleshooting of the perfusion systems described herein can be performed easily and quickly by an operator of the system.

In addition, human brains that are perfused using the perfusion systems and methods described herein can be tested to study disease mechanisms within the human brain. For example, brains exhibiting a particular disease or condition can be exposed to certain therapeutic agents (e.g., pharmaceutical compounds, antibodies, biologics, viral vectors, lipid nanoparticles, peptides, etc.) during perfusion using the perfusion systems and methods described herein, and the response of the brains to the therapeutic agents can be studied by performing testing on the brain and/or the perfusate in real-time during the perfusion process. In addition, brains can be mechanically or chemically altered prior to perfusion in order to simulate a particular disease mechanism. The mechanically or chemically altered brains can be exposed to certain therapeutic agents during perfusion using the perfusion systems and methods described herein, and the response of the brains to the therapeutic agents can be studied by performing testing on the brain and/or the perfusate in real-time during the perfusion process. In addition, individual mechanisms associated with particular brain diseases can be studied using the systems and methods described herein. For example, brains exhibiting a particular disease or altered to simulate the disease can be analyzed using the systems and methods described herein to identify and study particular compounds or molecules generated by the brain related to the disease or particular chemical or electrical activity of the brain related to disease.

The perfusion systems and methods described herein can also be used to determine the pharmacokinetics of therapeutic agents (also referred to herein as “drugs”), the pharmacodynamics of therapeutic agents, the likely efficacy of particular therapeutic agents on treating targeted diseases, and potential side effects that may result from administration of therapeutic agents. The perfusion systems and methods described herein can also be used to determine whether a therapeutic agent is able to cross the blood-brain barrier of the brain. As a result, the safety and efficacy of potential new drugs can be tested using the perfusate systems and methods described herein to accurately determine their likely effectiveness in treating the targeted diseases in humans without requiring administration of the drug to humans. For example, perfusion systems and techniques described herein provide a tightly controlled arterial input and high-dimension data sampling that allows investigators to expose the brain to any therapeutic modality in order to generate real and precise human multi-omic brain response networks required for phenotype-based drug discovery. The perfusion systems and techniques described herein can also provide highly differentiated capacity to determine safety profiles, pharmacological properties, and target engagement validation and deconvolution of desired therapeutics. The systems and methods described herein can be used to study the effects of drugs on the brain using non-diseased brains, diseased brains, or modeled brains.

The perfusion systems and techniques described herein can also be used to study mechanisms and potential treatments for brain injuries.

The accuracy of the data related to the brain captured using the systems and methods described herein has a high level of accuracy (e.g., a high signal to noise ratio) due to the isolation of the brain and the lack of influence from peripheral organs. The response of the brain to a particular stimulus can be isolated and determined with high levels of accuracy.

The details of certain implementations are set forth in the accompanying drawings and the description below. Other aspects, features, and advantages will be apparent from the description and drawings, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic illustration of a system for ex-vivo perfusion of a mammalian brain.

FIG. 2A is a perspective view of an example pulse generator of the system of FIG. 1.

FIG. 2B is an exploded view of the example pulse generator of FIG. 2A.

FIG. 2C is a side view of the example pulse generator of FIG. 2A.

FIG. 2D is a cross-sectional view of the pulse generator of FIG. 2A along axis A-A depicted in FIG. 2C.

FIG. 3 is a schematic illustration of a process of sampling perfusate and brain tissue during perfusion of a brain using the system of FIG. 1.

FIG. 4 is a schematic illustration of molecules in perfusate crossing the blood-brain barrier of a brain being perfused by the system of FIG. 1.

FIG. 5 depicts example Raman spectroscopy data indicating release profiles of rapamycin provided through perfusate of the perfusion system of FIG. 1.

FIG. 6 depicts example Raman spectroscopy data indicating the concentration of rapamycin in the tissue of a brain following introduction of rapamycin into the perfusate of the system of FIG. 1.

FIG. 7 depicts a schematic illustration of different mammalian brains perfused and tested using the system of FIG. 1.

FIG. 8 depicts example rates of lactate released by the mammalian brains depicted in FIG. 7 during perfusion of the brains using the system of FIG. 1.

FIG. 9 is a block diagram of an example computer system by which a computer system of the perfusion system of FIG. 1 can be implemented.

FIG. 10A is a perspective view of an example brain housing of the system of FIG. 1.

FIG. 10B is a side view of the example brain housing of FIG. 10A.

FIG. 10C is a bottom view of the example brain housing of FIG. 10A.

FIG. 10D is an exploded view of the example brain housing of FIG. 10A.

FIG. 10E is a perspective view of an example hemispherical brain chamber of the example brain housing of FIG. 10A.

FIG. 10F is a top view of the example hemispherical brain chamber of FIG. 10E.

FIG. 10G is a cross-sectional view of the example hemispherical brain chamber of FIG. 10E.

FIG. 11A is a perspective view of another example brain housing.

FIG. 11B is an exploded view of the example brain housing of FIG. 11A.

FIG. 11C is a side view of the example brain housing of FIG. 11A.

FIG. 11D is a cross-sectional view of the example brain housing of FIG. 11A.

FIG. 12A is a perspective view of an example brain interface configured to fluidly couple a brain to an organ line of the system of FIG. 1.

FIG. 12B is a side view of the example brain interface of FIG. 12A.

FIG. 12C is cross-sectional view of the example brain interface of FIG. 12A.

FIG. 13 shows an example control system.

FIG. 14 is a flow diagram of an example process for controlling an ex-vivo brain perfusion device using a controller machine learning model.

FIG. 15 is a flow diagram of an example process for training a controller machine learning model used to control an ex-vivo brain perfusion device.

FIG. 16 is an RNAScope image showing GFP and mCherry expressed in the frontal cortex of a brain.

FIG. 17A is a perspective view of the example brain housing stand of the system of FIG. 1.

FIG. 17B is a top view of the example brain housing stand of FIG. 17A.

FIG. 18A is a graph depicting adenosine levels of brains during a perfusion process.

FIG. 18B depicts a heat map of the ratio of total metabolome of the brain to the perfusate after 6 hours of perfusion and 24 hours of perfusion.

FIG. 19 is an RNAScope image showing AAV9 expressed in brain parenchyma of a perfused brain, without expression of AAV2 in the brain parenchyma.

FIG. 20A depicts a schematic of Parkinson's disease modeling by systemic MPTP administration to a brain perfused using the example system of FIG. 1.

FIG. 20B depicts a representative image of H&E staining of MPTP-treated brain tissue.

FIG. 20C depicts a graph showing reduction in plasma dopamine concentrations following systemic MPTP administration to a brain perfused using the example system of FIG. 1.

FIG. 21A depicts a graph showing principal component analysis (PCA) of the untargeted metabolomic data collected from perfusate samples collected from drug-treated and vehicle-treated brains 24 hours after perfusion using the example system of FIG. 1.

FIG. 21B depicts a volcano plot showing differential analysis of the untargeted metabolomic data collected from perfusate samples collected from drug-treated and vehicle-treated brains 24 hours after perfusion using the example system of FIG. 1.

FIG. 21C depicts a heatmap showing the metabolite abundances as measured in the untargeted metabolomic data collected from perfusate samples collected from drug-treated and vehicle-treated brains 24 hours after perfusion using the example system of FIG. 1.

FIG. 22A depicts graph showing principal component analysis (PCA) of the untargeted metabolomic data collected from prefrontal cortex (PFC) tissue samples collected from drug-treated and vehicle-treated brains 24 hours after perfusion using the example system of FIG. 1.

FIG. 22B depicts graph showing principal component analysis (PCA) of the untargeted metabolomic data collected from perfusate samples collected from drug-treated and vehicle-treated brains 24 hours after perfusion using the example system of FIG. 1.

FIG. 22C depicts a volcano plot showing differential analysis of the untargeted metabolomic data collected from prefrontal cortex (PFC) tissue samples collected from drug-treated and vehicle-treated brains 24 hours after perfusion using the example system of FIG. 1.

FIG. 22D depicts a volcano plot showing differential analysis of the untargeted metabolomic data collected from perfusate samples collected from drug-treated and vehicle-treated brains 24 hours after perfusion using the example system of FIG. 1.

FIG. 22E depicts a heatmap showing the metabolite abundances as measured in the untargeted metabolomic data collected from prefrontal cortex (PFC) tissue samples collected from drug-treated and vehicle-treated brains 24 hours after perfusion using the example system of FIG. 1.

FIG. 22F depicts a heatmap showing the metabolite abundances as measured in the untargeted metabolomic data collected from perfusate samples collected from drug-treated and vehicle-treated brains 24 hours after perfusion using the example system of FIG. 1.

FIG. 23A depicts a pathway analysis of metabolite changes as measured in the untargeted metabolomic data collected from perfusate samples collected from drug-treated and vehicle-treated brains 24 hours after perfusion using the example system of FIG. 1.

FIG. 23B depicts a mechanistic model for detecting oxidative stress and neuroinflammation by metabolite changes in perfusate samples collected using the example system of FIG. 1.

FIG. 24 depicts real-time metabolite monitoring of the mammalian brains depicted in FIG. 7 during perfusion of the brains using the system of FIG. 1.

FIG. 25 depicts time-resolved stable-isotope tracing of drug-treated and vehicle-treated brains during perfusion of the brains using the system of FIG. 1.

Like references symbols in the various drawings indicate like elements.

### DETAILED DESCRIPTION

This specification generally describes devices, systems, and methods for ex-vivo perfusion of mammalian organs. In particular, this specification describes devices, systems, and methods for perfusing a human brain for ex-vivo restoration and preservation of the human brain, including normal cellular activity of the brain, for two weeks or longer post-mortem or after global ischemia. In addition, this specification describes devices, systems, and methods for performing pharmacokinetic modeling and pharmacological testing using a perfused brain.

An example of an electromechanical perfusion device is described in: Zvonimir Vrselja et al., “Restoration of brain circulation and cellular functions hours postmortem,” Nature, 2019 April; 568(7752 ), which is incorporated by reference herein. Examples of electromechanical perfusion devices and artificial perfusion experiments are also described in U.S. patent application Ser. No. 16/967,925 and U.S. patent application Ser. No. 19/070,309, which are incorporated by reference herein.

Sustained, fully autonomous perfusion of ex vivo brain tissue requires a dynamically regulated environment that simulates the vascular-physics and metabolic feedback loops known to exist in the in vivo cerebral circulation. Cerebrovascular endothelial and mural cells transduce variations in wall shear stress, pulsatility, and apparent viscosity at successive arterial bifurcations and within the capillary network. As a result, deviations from the physiologically appropriate hemorheological gradient, which are characterized by higher effective viscosity and shear in large vessels and markedly lower values after shear-thinning in the microvasculature, can lead to maladaptive vasomotor responses, perturb mass-transfer of oxygen and glucose, and induce transcriptional reprogramming notwithstanding efficient elimination of soluble proteins and metabolites via the glymphatic system. Further, ongoing consumption of metabolic substrates and byproduct efflux drive time-dependent changes in perfusate viscosity, density, gas solubility, and electrolyte composition, which in turn drive responses in cerebrovascular endothelial cells.

To address these complex dynamics and enable automatic control of brain perfusion, the devices, systems, and methods described herein are configured to reflect human brain physiology and mass-transfer, integrate real-time rheological surveillance, and perform high-resolution hemodynamic modelling. In addition, the devices, systems, and methods described herein translate continuous bioenergetic function into a single functionally integrated adaptive control framework. Further, by iteratively updating perfusate mechanical properties, vascular-physics parameters, and metabolic set-points in a coordinated manner, the perfusion system described herein is configured to preserve cerebral homeostasis without physical intervention (e.g., fully autonomously), thereby enabling viable brain perfusion durations of two weeks of longer. In some implementations, the system is configured to control the viscosity, rheology, pulsatile flow, and oncotic pressure of the perfusate to closely match the characteristics of blood in order to ensure that the perfusate supports endothelial and metabolic functions. In addition, each of the mechanical components of the perfusion system described herein are sufficiently durable to withstand prolonged operation of the system (e.g., perfusion processes lasting two weeks or longer).

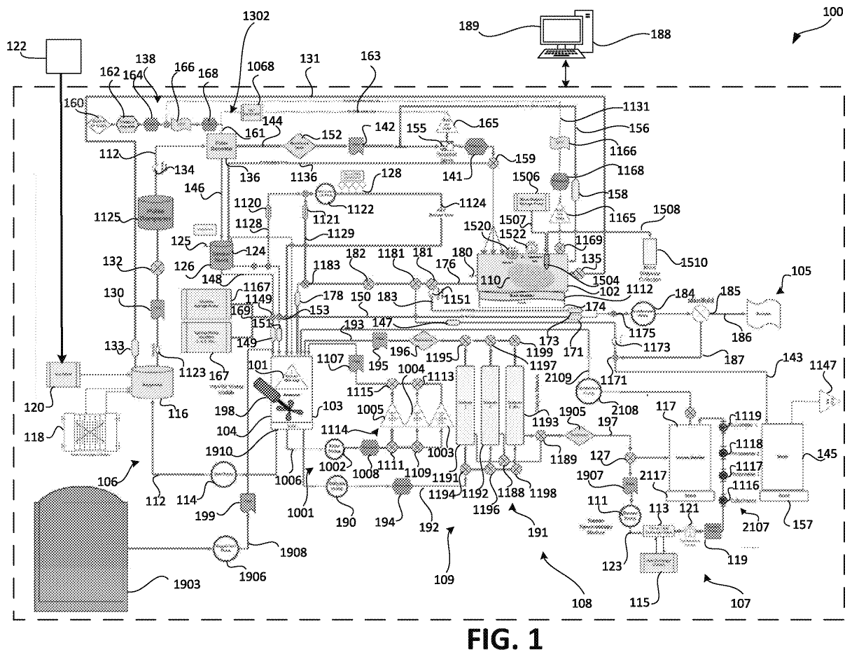

FIG. 1 depicts an example perfusion system 100 for perfusion, restoration, and preservation of a human brain 110. The perfusion system 100 includes a brain housing 102 that is configured to enclose a human brain 110 and a perfusate reservoir 104 that is configured to contain a perfusate solution (also referred to herein as “perfusate”). As will be described in further detail herein, the perfusate contained within the perfusate reservoir 104 is configured to perfuse and restore a human brain 110 contained inside the brain housing 102.

The brain housing 102 is fluidly connected to the perfusate reservoir 104 through an arterial circuit 106 and a venous circuit 108, which together form a closed-loop fluid circuit to circulate a synthetic acellular perfusate under a pulsatile flow.

The perfusate system 100 also includes a drain line 143 fluidly coupled to a waste container 145 configured to receive perfusate from the venous circuit 108. The waste container 145 can be, for example, a rigid, liquid tight container formed of glass or plastic. Alternatively, the waste container 145 can be a plastic bag. A solenoid valve 147 is positioned along the drain line 143 upstream of the waste container 145 and is configured to control flow of used perfusate to the waste container 145. The drain line 143 and the waste container 145 can be used to drain used perfusate from the system 100 without sending the used perfusate back to the perfusate reservoir 104. To drain perfusate from the system 100, the solenoid valve 147 along the drain line 143 is opened to allow perfusate flowing along the housing outlet line 176 to flow into the waste container 145. The waste container 145 is positioned on a waste scale 157 that is configured to record the weight of perfusate contained in the waste container 145.

At the beginning of the perfusion process, an initial flush of the brain 110 with perfusate is typically performed in order to remove toxins and other unwanted chemical compounds that have built up in the brain 110 during the post-mortem period prior to perfusion During the initial flush of the brain 110, the used perfusate flowing out of the brain 110 is sent to the waste container 145 rather than back to the perfusate reservoir 104. In order to sufficiently remove toxins and other unwanted chemical compounds that have built up in the brain 110 during the post-mortem period prior to perfusion, the brain 110 is flushed with a predetermined amount of perfusate that is not recirculated through the perfusate reservoir 104. During the initial flush of the perfusate process, the solenoid valve 178 along the housing outlet line 176 is closed and the solenoid valve 147 along the drain line 143 is open to direct used perfusate flowing out of the brain 110 along the drain line 143 to the waste container 145. Throughout the initial flush of the brain 110, the waste scale 157 measures the weight of used perfusate inside the waste container 145 in real-time. In response to the waste scale 157 detecting that a predetermined amount of used perfusate has been provided to the waste container 145 during the initial flush, the solenoid valve 178 along the housing outlet line 176 between the brain housing 102 and the perfusate reservoir 104 is opened and the solenoid valve 147 along the drain line 143 is closed, which directs used perfusate flowing along the housing outlet line back to the perfusate reservoir 104. In some implementations, the brain 110 is initially flushed with 500 mL of perfusate, as detected by the waste scale 157.

In some implementations, the drain line 143 and the waste container 145 are used to drain the venous circuit 108 at the end of a perfusion process. For example, at the end of the perfusion process, the solenoid valve 147 along the drain line 143 is opened, and the pump 114 is operated to flow perfusate from the brain housing 102 along drain line 143 into the waste container 145 to drain the brain housing 102, the perfusate reservoir 104, and the fluid lines of the venous circuit 108 of used perfusate.

In addition, the perfusate reservoir 104 is positioned on a scale 1910 and data generated by the scale 1910 can be used to detect fluid leaks within the perfusion system 100. For example, at the beginning of a perfusion treatment, the weight detected by the scale 1910 under the perfusion reservoir 104 can be combined with the weight detected by a scale 174 under the brain housing 102, a scale 2117 under a dialysis solution receptacle 117, and a scale 157 under a waste container 145 to record an initial perfusate weight. Throughout perfusion of the brain 110, the weights measured by each of the scales 1910, 174, 2117, 157 of the system 100 can be monitored in real-time, and a leak can be detected in the system 100 based on determining that the sum of the weights measured by each of the scales 1910, 174, 2117, 157 has deviated more than a threshold amount below initial perfusate weight.

The arterial circuit 106 includes an arterial fluid line 112 and an organ line 144 fluidly coupling the perfusate reservoir 104 to the brain housing 102, and the fresh perfusate contained within the perfusate reservoir 104 is provided to brain 110 inside the brain housing 102 along the arterial fluid line 112 and the organ line 144. The arterial circuit 106 also includes a flow pump 114 positioned along the arterial fluid line 112 between the perfusate reservoir 104 and an oxygenator 116. The flow pump 114 is a peristaltic pump configured to pump fresh perfusate from the perfusate reservoir 104 to the oxygenator 116. The peristaltic flow pump 114 is configured to minimize shear of the perfusate as it is pumped along the arterial fluid line 112. In some implementations, the flow pump 114 is configured to flow the perfusate to the oxygenator 116 at a rate of 400 mL/minute.

The oxygenator 116 includes a heat exchanger 118 and a gas mixer 120. The heat exchanger 118 includes one or more metal heat exchanging coils configured to regulate the temperature of the perfusate received by the oxygenator 116. As will be described in further detail herein, the arterial circuit 106 includes a sensor block 124 and sensor probes 128 positioned along a sensor line 1124 configured to measure certain characteristics of the perfusate flowing through the arterial circuit 106, including the temperature of the perfusate, and the heat exchanger 118 is configured to adjust the temperature of the perfusate flowing through the heat exchanger 118 based on the temperature of the perfusate detected by the sensor probes 128. For example, the heat exchanger 118 is configured to adjust the temperature of the perfusate to be within a predetermined range of temperature based on the temperature signals generated by a temperature sensor of the sensor probes 128. For example, during perfusion, a solenoid valve 1120 along an arterial sensor line 1128 is open to fluidly couple the sensor line 1124 to the sensor block 124 and a peristaltic sensor pump 1122 is operated to draw perfusate from the sensor block 124 along the sensor line 1124 and through the sensor probes 128. The temperature of the perfusate flowing along the sensor line 1124 is measured by a temperature sensor of the sensor probes 128. The heat exchanger 118 can be automatically controlled to adjust the temperature of the perfusate measure by the sensor probes 128. As will be described in further detail herein, at the beginning of treatment, the temperature of the perfusate in the system 100 is in a range of 16 °C to 20 °C and the heat exchanger 118 can be used to increase the temperature of the perfusate at a controlled rate at the beginning of the perfusion process. In some implementations, the heat exchanger 118 is configured to increase to temperature of the perfusate to be within a range of 35 °C to 37 °C.

The gas mixer 120 of the oxygenator 116 is fluidly coupled to and receives one or more gasses from a gas source 122, and the gas mixer 120 is configured to dissolve the one or more gasses into the perfusate flowing through the oxygenator 116. The gas mixer 120 includes three inlets fluidly coupled to the gas source 122. A first inlet of the gas mixer 120 is configured to be fluidly coupled to a source of O₂, a second inlet of the gas mixer 120 is configured to be coupled to a source of N₂, and a third inlet of the gas mixer 120 is configured to be coupled to a source of CO₂. The gas mixer 120 is controlled based on one or more signals generated by the sensor probes 128 indicating the dissolved gas levels of the perfusate. For example, the sensor probes 128 can generate one or more signals indicating the dissolved oxygen concentration of the perfusate withdrawn from the sensor block 124 and flowing along the sensor line 1124, the dissolved nitrogen concentration of the perfusate withdrawn from the sensor block 124 and flowing along the sensor line 1124, and the dissolved carbon dioxide concentration of the perfusate withdrawn from the sensor block 124 and flowing along the sensor line 1124 and, based on the signals, the gas mixer 120 adjusts the amount of oxygen, nitrogen, and/or carbon dioxide provided to the perfusate within the oxygenator 116 in order to control the dissolved oxygen concentration, the dissolved nitrogen concentration, and the dissolved carbon dioxide concentration of the perfusate to be within a predetermined range. For example, based on the signals generated by the sensor probes 128, the gas mixer 120 generates commands to control internal gas valves of the gas mixer 120 to control the flow rate of gas into each respective inlet of the gas mixer 120 in order to control the amount and rate that each gas (O₂, N_{2,} and CO₂) are provided to the oxygenator 116. In some implementations, the system is configured to maintain the oxygen concentration of the perfusate within 10% of a target oxygen saturation. In some implementations, the partial pressure of oxygen is measured every 100 s and if the change in partial pressure of O₂ (ΔPO₂) exceeds a threshold value (e.g., (ΔPO₂≤−20%), indicating simulated hypoxia, an alert is triggered.

In addition, the gas mixer 120 monitors the pressure and flow rate of gas provided from the gas mixer 120 to the oxygenator 116. In some implementations, the gas mixer 120 maintains the flow rate of gas from the gas mixer 120 to the oxygenator 116 at approximately 700 mL/minute. As described herein, in some implementations, the flow pump 114 is configured to flow the perfusate to the oxygenator 116 at a rate of 400 mL/minute. By flowing the gas into the oxygenator 116 at a rate of 700 mL/minute and simultaneously flowing perfusate into the oxygenator 116 at a rate of 400 mL/minute, a predetermined ventilation/perfusion coefficient of 700/400 can be maintained throughout the perfusion process. By adjusting the flow rate of each of the gasses flowing into the gas mixer 120 while maintaining the rate of gas flowing out of the gas mixer 120 into oxygenator 116, the₂ gas mixer 120 can control and adjust the concentration of each of the respective gasses within the perfusate while maintaining the predetermined ventilation/perfusion coefficient.

The concentration of the CO₂, N₂, and O₂ within the perfusate are maintained within physiological limits, as determined based on the concentration detected using the sensor probes 128 in the sensor block 124. In some implementations, the gas mixer 120 and oxygenator 116 are controlled based on an alpha-stat approach to maintain the blood gas concentrations within normal, physiological limits. In some implementations, the gas mixer 120 and oxygenator 116 are controlled based on a pH-stat approach to maintain the blood gas concentrations within normal, physiological limits. In some implementations, the partial pressure of the CO₂ within the perfusate is maintained within a range 20 mmHg to 50 mmHg during the perfusion process. In some implementations, the partial pressure of O₂ in the perfusate is maintained within a range of 350 mmHg to 400 mmHg during the perfusion process. As will be described in further detail herein, at the beginning of a perfusion procedure, the temperature of the perfusate is gradually increased by the heat exchanger 118 and the O₂ concentration of the perfusate is gradually increased by the gas mixer 120 until a target temperature and O₂ concentration for the perfusate are reached, as detected based on the sensor probes 128 in the sensor block 124. For example, at the beginning of perfusion, the partial pressure of O₂ in the perfusate is 200 mmHg and the gas mixer 120 and oxygenator 116 are controlled to gradually increase the partial pressure of O₂ in the perfusate from 275 mmHg to 375 mmHg. By gradually increasing the temperature and the O₂ concentration of the perfusate, the internal resistance of the brain 110 is decreased, which results in improved perfusion of the brain 110 and reduced risk of injury to the brain 110.

In some implementations, the oxygenator 116 is configured to exhibit no structural failure or substantial pressure increase throughout a prolonged perfusion process (e.g., 2 weeks or longer of operation). In addition, in some implementations, the oxygenator 116 is controlled by a proportional-integrative-derivative (PID) controller, and the oxygenator 116 maintains a 95% or greater PID response efficiency to changes in the concentration of the CO₂, N₂, and O₂ throughout a prolonged perfusion process (e.g., 2 weeks or longer of operation).

A shunt line 131 is coupled to and extends from the oxygenator 116 and fluidly couples the oxygenator 116 to the brain housing 102. The shunt line 131 is configured to carry fresh perfusate out of the oxygenator 116 directly to the brain housing 102 without passing through a pulse generator 136 of the perfusate system 100. The shunt line 131 can be used in preparation stages to quality check the device prior to the connection of the brain. The shunt line 131 can be used to provide perfusate to the brain housing 102 during the quality check period but is generally not used during the primary application of brain perfusion. A stopcock 135 is positioned along the shunt line 131 downstream of the oxygenator 116. The stopcock 135 can be used to fluidly coupled the shunt line 131 to the brain housing 102 and to sample the perfusate flowing out of the oxygenator 116 prior to providing the perfusate to the brain 110, for example, in order to conduct tests on the perfusate following oxygenation of the perfusate by the oxygenator 116.

A solenoid valve 133 is positioned along the shunt line 131 and is configured to control the flow of perfusate between the oxygenator 116 and the brain housing 102 along the shunt line 131. For example, during the initial set up of the perfusate system 100 for a perfusate procedure, the stopcock 135 is controlled (e.g., using a servo-motor) to fluidly couple the shunt line 131 to the brain housing 102, the solenoid valve 133 along the shunt line 131 is opened and the flow pump 114 is operated to gradually increase the flow of perfusate through the oxygenator 116 to 400 mL/minute, and perfusate flows through the oxygenator 116 along the shunt line 131 and into the brain housing 102. As perfusate flows through the oxygenator 116 and the shunt line 131, any air in the fibers of the oxygenator 116 is forced out of the oxygenator 116 along the shunt line 131 to the brain housing 102, which allow for gas exchange into the perfusate in the oxygenator 116. Once all of the air has been flushed out of the oxygenator 116, the solenoid valve 133 is closed to prevent flow of perfusate from the oxygenator 116 to the brain housing 102 along the shunt line 131.

The arterial circuit 106 includes a reducer 1123 configured to fluidly couple the oxygenator 116 to the portion of the arterial line 112 downstream of the oxygenator 116. The arterial circuit 106 include also includes a flow sensor 130 positioned along the arterial fluid line 112 downstream of the oxygenator 116. When the solenoid valve 133 along the shunt line 131 is closed and the flow pump 114 is operated, fresh perfusate exiting the oxygenator 116 flows along the arterial fluid line 112 through the flow sensor 130, and the flow pump 114 is controlled based on signals generated by the flow sensor 130. In some implementations, the pump speed of the flow pump 114 is adjusted based on one or more signals received from the flow sensor 130 indicating the flow rate of the perfusate exiting the oxygenator 116 in order to control the flow rate of the perfusate exiting the oxygenator 116 to be within a predetermined range. In some implementations, the flow pump 114 is controlled to maintain the flow rate of perfusate exiting the oxygenator 116 to be 400 mL/minute. As previously discussed, the flow rate of perfusate into the oxygenator 116 and the flow rate of gas into the oxygenator 116 can each be controlled to maintain a predetermined ventilation/perfusion coefficient. Thus, in some implementations, the flow pump 114 and the gas mixer 120 are each controlled to adjust the flow rates of perfusate and gas, respectively, into the oxygenator 116 in order to maintain the predetermined ventilation/perfusion coefficient.

Still referring to FIG. 1, the arterial circuit 106 includes a stopcock 132 positioned along the arterial fluid line 112 downstream of the oxygenator 116. The stopcock 132 can be used to sample the perfusate flowing out of the oxygenator 116, for example, in order to conduct tests on the perfusate following oxygenation of the perfusate by the oxygenator 116.

The arterial circuit 106 also includes a one-way valve 134 positioned downstream of the oxygenator 116 and the flow sensor 130. The one-way valve 134 is configured to prevent perfusate in upstream portions of the arterial fluid line 112 from flowing back into the oxygenator 116.

The arterial circuit 106 also includes a pulse generation system 1302 configured to convert laminar flow of perfusate along the arterial fluid line 112 to pulsatile flow along the organ line 144. The pulse generation system 1302 includes a pulse dampener 1125, a pulse generator device 136 (also referred to herein as “pulse generator 136”) and an air supply system 138 fluidly coupled to the pulse generator 136.

After flowing out of the oxygenator 116, the perfusate flows along the arterial fluid line 112 through the pulse dampener 1125. The pulse dampener 1125 includes a pressurized air chamber that reduces the oscillation of the fluid flow generated by the peristaltic flow pump 114, which reduces oscillatory flow disturbances in the organ line 144. For example, as will be described in detail, the pulse generation system 1302 includes a flow sensor 142 and a pressure sensor 141 that are configured to measure the flow rate and pressure, respectively, of perfusate flowing along the organ line between the pulse generator and the brain housing 102. Fast Fourier Transform algorithms can be applied to the signals generated by the flow sensor 142 and the pressure sensor 141 in order to detect variances (noise) in the pressure and flow rate of perfusate along the organ line 144 associated with actuation of the peristaltic flow pump 114. The pulse dampener 1125 can be dynamically controlled based on the data received from the flow sensor 142 and the pressure sensor 141 in order to reduce the variance in pressure and flow of perfusate along the organ line 144. In some implementations, the pulse dampener 1125 is controlled to maintain the variance in pressure and flow rate of the perfusate flowing to the pulse generator 136 within a target frequency range.

After flowing out of the pulse dampener 2116, the perfusate flows along the arterial fluid line 112 through the one-way valve 134 to the pulse generator 136. The pulse generator 136 is configured to convert the steady state flow of the fresh perfusate along the arterial fluid line 112 to pulsatile flow. As a result, fresh perfusate is provided to the brain 110 as a pulsatile flow that more closely mimics the anatomical flow of blood to the human brain, which results in improved perfusion of the brain 110. As will be described in detail herein, the pulsatile flow of perfusate generated by the pulse generation system 1302 is dynamically adjusted throughout the perfusion process to optimize the flow rate and fluid pressure of perfusate provided to the brain 110 responsive to the level of resistance of the brain 110, which improves longevity of the cellular and metabolic activity and tissue of the brain 110 being perfused by the system 100.

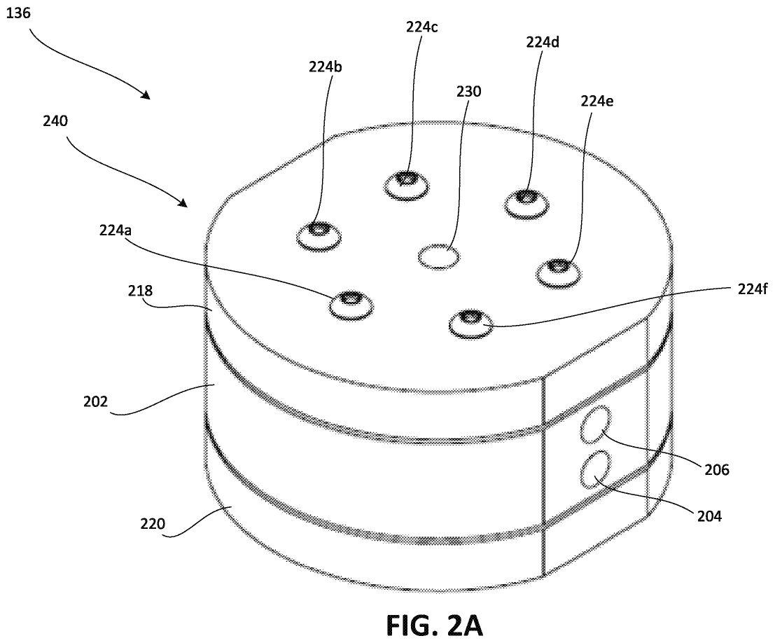

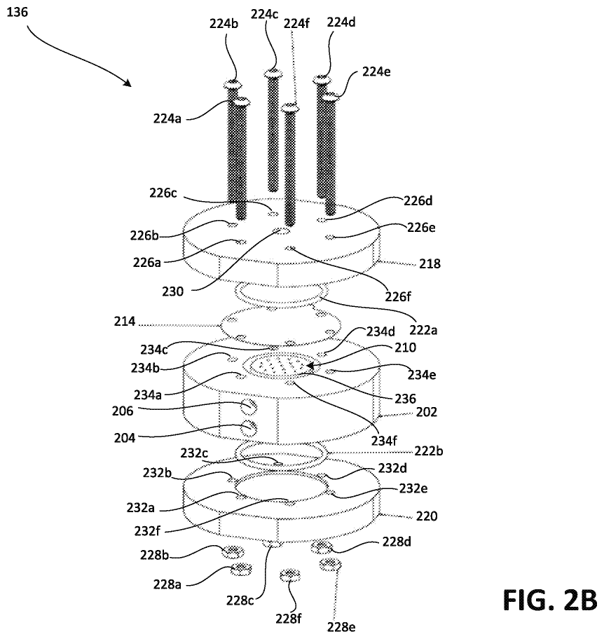

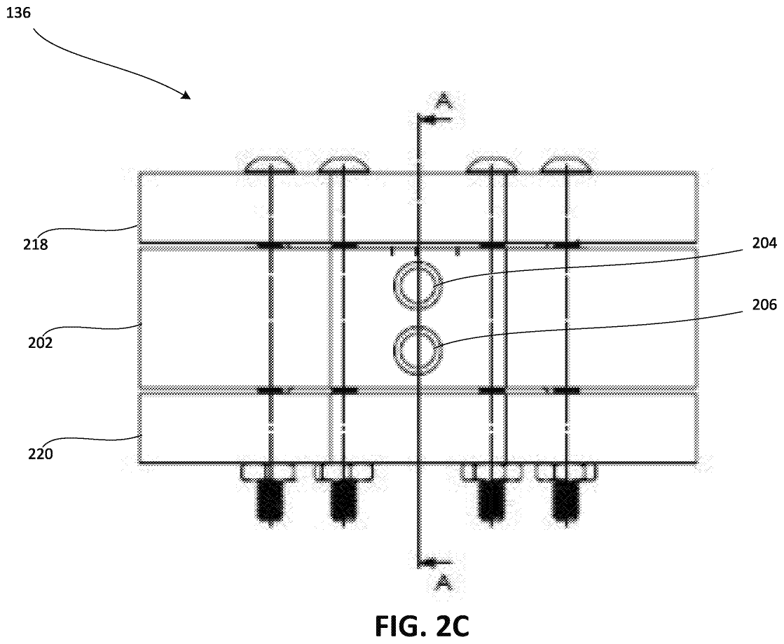

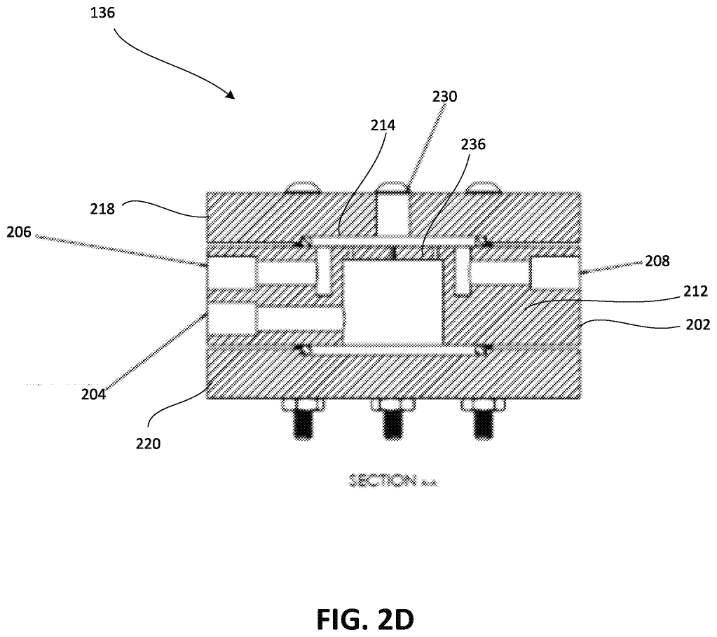

FIGS. 2A-2D depict the example pulse generator device 136. The pulse generator 136 includes a body 240 that defines a perfusate inlet 204, a sensor block outlet 206, and an organ line outlet 208. Referring to FIGS. 1 and 2A-2D, the perfusate inlet 204 is fluidly coupled to a fluid chamber 236 of the pulse generator 136. The fluid chamber 236 is configured to receive perfusate flowing into the pulse generator 136 from the arterial fluid line 112 through the perfusate inlet 204. The fluid chamber 236 includes a plurality of openings 210 through an upper surface of the fluid chamber 236, and perfusate received by the fluid chamber 236 from the perfusate inlet 204 is configured to flow through the openings 210 into the interior 212 of a central housing 202 of the pulse generator 136. Fluid contained within the interior 212 of the central housing 202 flows through the sensor block outlet 206 and/or the organ line outlet 208, as will be described herein.

Referring to FIG. 2B, the pulse generator 136 further includes a lid 218 and a base 220. A flexible diaphragm 214 is positioned over the fluid chamber 236 between the lid 218 and the central housing 202 of the pulse generator 136. The lid 218 is coupled to an upper surface of the central housing 202 and the base 220 is coupled to a lower surface of the central housing 202. O-rings 222a, 222b are positioned between the lid 218 and the central housing 202 and between the base 220 and the central housing 202, respectively, in order to prevent perfusate contained within the interior 212 of the central housing 202 from leaking outside of the central housing 202. The lid 218 and the base 220 are coupled to the central housing 202 by a plurality of bolts 224a, 224b, 224c, 224d, 224e, 224f (collectively referred to herein as bolts 224) inserted through a series of respective openings 226a, 226b, 226c, 226d, 226e, 226f through the lid 218, respective openings 234a, 234b, 234c, 234d, 234e, 234f through the central housing 202, and respective openings 232a, 232b, 232c, 232d, 232e, 232f through the base 220. A respective plurality of nuts 228a, 228b, 228c, 228d, 228e, 228f are to the bolts 224.

The lid 218 of the pulse generator 136 defines an air inlet 230 fluidly coupled to and configured to receive pressurized air from the air supply system 138. The air supply system 138 is configured to provided pressurized air to the pulse generator 136 in order to control the pulsatile flow of perfusate received by pulse generator 136. For example, the diaphragm 214 flexes downwards towards the fluid chamber 236 in response to pressurized air being provided by the air supply system 138 through the air inlet 230 of the pulse generator 136. The flexure of the diaphragm 214 causes the openings 210 in the fluid chamber 236 to be covered by the diaphragm 214 and increases the pressure within the interior 212 of the central housing 202, which results in increased flow through the organ line outlet 208 of the pulse generator 136 to the organ line 144 that fluidly couples the pulse generator to the brain 110.

In some implementations, the diaphragm 214 is configured to retain greater than 95% amplitude fidelity after 24 hours or more of continuous operation of the pulse generator 136. In some implementations, the diaphragm 214 exhibits actuation waveform deviation of 5% or less from initial calibration after 24 hour or more of continuous operation of the pulse generator 136. The diaphragm is also configured to exhibit no visible mechanical fatigue, leaks, or pressure irregularities during continuous operation of the pulse generator 136 for 24 hours or longer. In some implementations, the diaphragm 214 is a back pressure regulator membrane manufactured by EQUILIBAR®.

Referring to FIG. 1, the air supply system 138 includes an air source 160, a first pressure regulator 162, a first pressure sensor 164 positioned downstream of the first pressure regulator 162, a second pressure regulator 166 positioned downstream of the first pressure regulator 162, and a second pressure sensor 168 positioned downstream of the second pressure regulator 166. The air source 160, the first pressure regulator 162, the second pressure regulator 166, and the pulse generator 136 are each fluidly coupled along an air supply line 161.

The first pressure regulator 162 is to provide a pressurized stream of air in a particular pressure range to the second pressure regulator 166, which is an electronic pressure sensor. In some implementations, the first pressure regulator 162 is configured to regulate the air supply 160 in order to provide a stream of pressurized air to the second pressure regulator 166 that is in a range of 1 to 5 psi. The first pressure sensor 164 can be used to confirm the pressure of the air stream generated by the first pressure regulator 162 to ensure that the air stream provided to the second pressure regulator 166 is within the appropriate pressure range.

The second pressure regulator 166 is an electronic pressure regulator that has tighter tolerances compared to the first pressure regulator 162 and is configured to provide a pressurized air stream within a particular pressure range to the air inlet 230 of the pulse generator 136. In some implementations, the second pressure regulator 166 is configured to generate an air stream with a pressure of 5 psi. The pressure of the air stream generated by the second pressure regulator 166 is controlled based on pressure signals received from the second pressure sensor 168 of the air supply system 138. In addition, as will be described in further detail herein, the second pressure regulator 166 is controlled based on signals received from a pressure sensor 141 and a flow sensor 142 positioned along the organ line 144 that fluidly couples the pulse generator 136 to the brain 110 inside the brain housing 102. The use of two electronic pressure regulators 162, 166 to regulate the pressure of the air stream provided to the pulse generator 136 results in a smoother and more controlled supply of pressurized air to the pulse generator 136 compared to using a single electronic pressure regulator. In some implementations, the one or more of the pressure regulators 162, 166, 1166 is a QPV electronic pressure regulator manufactured by EQUILIBAR®.

The air supply system 138 also includes an air calibration line 163 that fluidly couples the air supply line 161 to the organ line 144 upstream of the pressure sensor 141 positioned along the organ line 144. As will be described in further detail, the pressure sensor 141 along the organ line 144 is calibrated by providing pressurized air to the organ line 144 along the air calibration line 163. A filter 165 is positioned along the air calibration line 163 in order to filter out and prevent any particulates in the pressurized air stream flowing along the air calibration line 163 from entering the organ line 144. In some implementations, the filter 165 is a 0.22 μm filter.

In order to restore and maintain the cellular and metabolic activity of the brain 110, the pulse generator 136 and the air supply system 138 are controlled to provide a pulsatile flow of fresh perfusate to the brain 110 within predetermined boundaries of pressure and flow rate that are selected to effectively perfuse the brain while minimizing damage to the vasculature and other tissues of the brain 110. Referring to FIGS. 1 and 2D, a sensor block inlet line 146 is coupled to the sensor block outlet 206 of the pulse generator 136 to fluidly couple the pulse generator 136 to the sensor block 124. The organ line 144 is coupled to the organ line outlet 208 of the pulse generator 136 to fluidly couple the pulse generator 136 to the brain 110. The sensor block inlet line 146 has a larger diameter than the organ line 144. In addition, the organ line 144 and the pulse generator 136 are each positioned on a respective platform that maintains the brain housing 102, the organ line 144, and the pulse generator 136 at substantially equal heights above the height of the sensor block inlet line 146.

In some implementations, the sensor block inlet line 146 is a y-splitter line with a single line flowing out of the pulse generator 136 that splits into two fluid lines flowing into the sensor block 124. The sensor block inlet line 146 extends downwards relative to the organ line 144. As a result of the relative positioning of the pulse generator 136, the brain housing 102, the organ line 144, and the sensor block 124, there is no passive flow of fluid from the pulse generator 136 to the brain 110 inside the brain housing 102, and all of the perfusate contained within the interior 212 of the central housing 202 of the pulse generator 136 passively flows through the sensor block inlet line 146 when the diaphragm 214 of the pulse generator 136 is in an unflexed (“neutral”) position. Therefore, in order to provide fresh perfusate from the pulse generator 136 to the brain 110 via the organ line 144, the diaphragm 214 of the pulse generator 136 must be flexed downwards towards the fluid chamber 236 to cover the openings 210 in the fluid chamber 236 and increase the pressure within the interior 212 of the central housing 202. As result of the increased pressure caused by flexing the diaphragm 214, at least a portion of the fresh perfusate contained within the interior 212 of the pulse generator is forced through the organ line outlet 208 and along the organ line 144 to the brain 110.

In addition, the sensor block inlet line 146 has a larger diameter than the organ line 144. In some implementations, the sensor block inlet line 146 includes a Y-connector and two fluid lines extending from the Y-connector to fluidly couple the pulse generator 136 to the sensor block 124, further decreasing the resistance to flow between the pulse generator 136 and the sensor block 124. As a result of the decreased resistance to flow between the pulse generator 136 and the sensor block 124 compared to the resistance to flow between the pulse generator 136 and brain 110 inside the brain housing 102 due to the structural differences in the sensor block inlet line 146 and the organ line 144, at least a portion of the perfusate flowing through the pulse generator 136 is provided to the sensor block 124 from the pulse generator 136, even when the diaphragm 214 of the pulse generator 136 is in a flexed position.

In order to maintain an optimal pulsatile flow of perfusate along the organ line 144 that is responsive to the internal resistance of the brain 110, the air supply system 138 coupled to the pulse generator 136 is controlled based on signals generated by a pressure sensor 141 and a flow sensor 142 positioned along the organ line 144. In some implementation, the pulse generation system 1302 is controlled to maintain the pressure along the organ line 144, as measured by the pressure sensor 141, between 5 mmHg and 100 mmHg. In some implementation, the pulse generation system 1302 is controlled to maintain the flow rate along the organ line 144, as measured by the flow sensor 142, between 0 mL/min and 750 mL/minute. The flow rate measured by the flow sensor 142 corresponds to the average flow rate of the pulse of fluid provided by the pulse generator 136.

The second pressure regulator 166 of the air supply system 138 is controlled to provide pressurized air to the air inlet 230 of the pulse generator 136 at particular intervals and durations based on the signals generated the pressure sensor 141 and the flow sensor 142 along the organ line 144 in order to achieve a particular pressure and/or flow rate of perfusate along the organ line 144. As the second pressure regulator 166 provides pressurized air to the air inlet 230 of the pulse generator 136, the diaphragm 214 of the pulse generator 136 flexes downwards towards the fluid chamber 236 in response to the increased positive pressure in the air inlet 230 and covers the openings 210 in the fluid chamber 236. As result of the increased pressure within the interior 212 of the pulse generator 136 caused by the flexing of the diaphragm 214, at least a portion of the fresh perfusate contained within the interior 212 of the pulse generator is forced through the organ line outlet 208 and along the organ line 144 to the brain 110. By adjusting the frequency and/or duration of the pressurized air streams provided by the second pressure regulator 166 to the pulse generator 136 in response to pressure and flow rate signals received from sensors 141, 142, the amplitude and base of the pulse of perfusate provided by the pulse generator 136 to the organ line 144 can be optimized based on the internal resistance of the brain detected by the sensor 141, 142.

In addition, as depicted in FIG. 1, the perfusion system 100 includes a resistance valve 152 positioned along the organ line 144 and that can be operated to further provide a particular flow resistance along the organ line 144. In particular, the resistance valve 152 is controlled to prevent passive flow of perfusate along the organ line 144 due to the driven flow provided by the flow pump 114.

The resistance valve 152 is positioned along the organ line 144 upstream of the flow sensor 142 and the pressure sensor 141. During calibration of the system 100, the flow pump 114 is operated to flow perfusate along the organ line 144 toward the brain 110 at a predetermined rate without operating the pulse generation system 1302. As the flow pump flows perfusate along the organ line 144 during calibration, the pressure along the organ line 144 downstream of the resistance valve 152 is measured by the pressure sensor 141. Based on the pressure measured by the pressure sensor 141, the resistance valve 152 can be adjusted (e.g., partially or fully opened or partially or fully closed) in order to predetermined internal resistance along the organ line 144. In some implementations, the resistance valve 152 is controlled, based on the data generated by the pressure sensor 141 to provide an internal resistance of 1 mmHg along the organ line 144.

During the post-mortem interval prior to beginning the perfusion process, the brain 110 undergoes processes resulting in an increased internal resistance to flow within the vasculature of the brain 110. For example, during the post-mortem interval prior to beginning the perfusion process, water leaks into the brain tissue and collapses the capillaries of the brain 110, resulting in the formation of internal Starling resistors in the brain and an increased internal resistance to flow within the vasculature of the brain 110. Therefore, in order to condition the vasculature of the brain 110 to sufficiently uptake the fresh perfusate and perfuse the brain, the pulse generator 136 is controlled at the beginning of the perfusion process to gradually increase the pressure and flow rate of the fresh perfusate provided along the organ line 144 in a stepwise manner based on signals received from the pressure sensor 141 and the flow sensor 142 positioned along the organ line 144. For example, at the beginning of the perfusion process, the pressure regulators 162, 166 of the air supply system 138 are controlled to provide pressurized air to the pulse generator 136 and cause the pulse generator 136 to increase the pressure of the pulsatile flow of the fresh perfusate along the organ line 144 until an initial pressure threshold is detected by the pressure sensor 141. In some implementation, the initial pressure threshold along the organ line is 16 mmHg. Once the initial pressure threshold along the organ line 144 has been maintained for a predetermined period, the electronic pressure regulator 166 of the air supply system 138 is controlled to cause the pulse generator 136 to increase the pulsatile flow along the organ line 144 until a second pressure threshold is detected along the organ line 144 by the pressure sensor 141.

In some implementations, the air supply system 138 and the pulse generator 136 are controlled based on signals received from the pressure sensor 141 to increase the pressure along the organ line 144 at a rate of 0.0333 mmHg/minute. In some implementations, the signals generated by the pressure sensor 141 and the flow sensor 142 are provided to a machine learning model that is trained to detect and predict internal resistance in the brain 110, and the air supply system 138 and the pulse generator 136 to increase the pressure along the organ line 144 at a rate determined by the machine learning model to be optimal for reducing internal resistance of the brain 110. By controlling the pulse generation system 1302 to gradually increase the pressure of perfusate provided to the brain 110, the internal resistance of the brain 110 is correspondingly gradually reduced, which enables the vasculature of the brain 110 to more effectively receive the perfusate while minimizing injury or other damage to the brain 110.

As the pressure along the organ line 144 is increased, the flow rate of perfusate along the organ line 144 is simultaneously monitored using the flow sensor 142, and the pulse generation system 1302 is controlled to continually increase the pressure along the organ line 144 until a target flow rate is detected along the organ line 144. In some implementations, the target flow rate is 10 mL/minute. Once the target flow rate is detected by the flow sensor 142, the pulse generation system 1302 is controlled based on signals generated by the flow sensor 142 in order to provide pulsatile flow along the organ line 144 sufficient to maintain the flow rate along the organ line 144 as detected by the flow sensor 142.

In some implementations, the target flow rate along the organ line 144 is a predetermined flow rate. In some implementations, data generated in real time by one or more sensors of the system 100 (e.g., sensor probes 128, Raman probe 119, colorimeter 1116, photometers 1117, 1118, fluorimeter 1119) is provided to a machine learning model that is trained to detect and predict the health of a brain 110 being perfused by the system 100 based on the sensor data, and the target flow rate along the organ line 144 is adjusted based on the output of the machine learning model in order to improve health of the brain tissue and extend the potential length of perfusion of the brain 110.

The pulse generation system 1302 is controlled throughout the perfusion period (e.g., 24 hours or longer, 48 hours or longer, 1 week or longer, 2 weeks or longer, etc.) based on the signals generated by the pressure sensor 141 and the flow sensor 142 in order to maintain the target flow rate of perfusate along the organ line 144 throughout the perfusion process while also maintaining the pressure along the organ line 144 within the target pressure boundaries. For example, while perfusate is provided to the brain 110 in pulses generated by the pulse generation system 1302 to maintain a target flow rate, the pressure along the organ line 144 continues to be measured by the pressure sensor 141. If the pressure detected by the pressure sensor 141 falls outside of target pressure boundaries, the pulse generation system 1302 is controlled to adjust the pulsatile flow along the organ line 144 is adjusted so that the pressure measured by the pressure sensor 141 is within the target pressure boundaries. Because the pulsatile flow provided by the pulse generator 136 to the brain 110 is controlled in real-time based on the pressure and the flow rate detected by the pressure sensor 141 and the flow sensor 142, respectively, the flow of perfusate to the brain 110 is controlled dynamically in response to the real-time changes in internal resistance experienced by the brain 110 throughout the perfusion process.

As used herein, a real-time operation may describe an operation that is performed with minimal delay, taking into account the limitations of the computing system(s) performing the operation. For example, in some implementations, the flow sensor 142 is sampled at a rate of 1 kHz or greater, the pressure sensor 141 is sampled at a rate of 300 Hz or greater, and the pulse generation system 1302 is controlled adjust the flow of perfusate to the brain every 100 milliseconds (or more frequently) based on the data received from the flow sensor 142 and the pressure sensor 141. In some implementations, the pressure sensor 141 is sampled at a rate of 1 kHz or greater. In some implementations, the flow sensor 130 is sampled at a rate of 1 kHz or greater and the flow pump 114 is controlled every 100 milliseconds (or more frequently) based on the data generated by the flow sensor 130. In some implementations, the sensor probes 128 along the sensor line 1124 and the sensor(s) in the sensor block 124 are each sampled at a rate of 1 kHz or greater, and the operations of the heat exchanger 118, the gas mixer 120, and the syringe pump manifolds 167, 1167 are each updated every 100 milliseconds (or more frequently) based on the data generated by the sensor probes 128 and sensor(s) in the sensor block. In some implementations, the resistance valve 152 is controlled at a 1 kHz or greater frequency. The dynamic nature of the perfusate flow provided to the brain 110 by the pulse generation system 1302 ensures that the brain 110 is sufficiently perfused while simultaneously preventing injury to the brain 110.

The pressure sensor 141 and the flow sensor 142 are each high speed data acquisition devices. For example, in some implementations, the pressure sensor 141 and the flow sensor 142 are each sampled at a rate of 1kHz or greater, the pressure sensor 141 and the flow sensor 142 each has a resolution of 0.1 mmHg (or less), and the pressure sensor 141 and the flow sensor 142 each has an error rate (e.g., signal noise) of 2% or less under dynamic pressure trials.

In some implementations, the mechanical actuation components of the system 100 (e.g., pumps 111, 114, 184, 190, 1002, 1122, 1906, 2108, the pressure regulators 162, 166, the resistance valves 152, 196, 1905, and the pulse generator 136, etc.) are controlled, based on sensor feedback, a central programmable logic controller (PLC). In some implementations, the actuation response time of the system 100 (e.g., the time elapsed between sensor signal detection and updated mechanical component actuation commands) is 100 milliseconds for less. In addition, the output variability of the system 100 can be maintained within ±5% of target values during perfusion. For example, the target flow rate, pressure, and pulse amplitude along the organ line 144 can maintained with ±5% variability across 3 or more sequential trials of the perfusion system 100. In some implementations, the pressure along the organ line 144 is maintained within ±5% of target pressure setpoints for 5 minutes or longer during dynamic trials of the perfusion system 100. In some implementations, the pulse waveform generated by the pulse generation system 1302 deviates 2% or less from a target signal profile throughout during real-time modulation of the system 100.

As previously discussed, at least a portion of the fluid flowing through the pulse generator 136 is provided to the sensor block 124 along the sensor block inlet line 146. The sensor block 124 includes a housing 126 configured to receive and contain fresh perfusate flowing from the pulse generator 136. The flow rate of perfusate into and out of the sensor block 124, the volume of perfusate flowing into and out of the sensor block 124, and the times at which perfusate is added to and drained from the sensor block 124 can be monitored and recorded.

The sensor block 124 also includes one or more sensor probes, including a viscometer, that are positioned within the housing 126 of the sensor block 124. The sensor probes in the sensor block 124 are configured to measure certain characteristics of the fresh perfusate inside the housing 126, including viscosity. In some implementations, based on detecting that the viscosity of the perfusate is outside a target range using the viscometer in the sensor block 124, the system 100 is automatically controlled to adjust the viscosity of the perfusate until the viscosity of the perfusate is within the target range.

For example, the perfusion system 100 includes a supplementary fresh perfusate reservoir 1903 that contains a supply of base perfusate solution that is maintained within a predetermined temperature range. The supplementary fresh perfusate reservoir 1903 is fluidly coupled to the perfusate reservoir 104 along a fresh perfusate line 1908 and a supplement pump 1906 is configured to pump fresh perfusate fluid contained within the fresh perfusate reservoir 1903 into the perfusate reservoir 104 along a fresh perfusate line 1908. As fresh perfusate is added from the supplementary perfusate reservoir 1903 to the perfusate reservoir 104, a solenoid valve 147 along a drain line 143 is simultaneously opened to direct a portion of the perfusate flowing through the venous circuit to a waste container 145. As depicted in FIG. 1, the waste container 145 includes a filter 1147 configured to filter perfusate solution flowing into the waste container 145. In some implementations, the filter 1147 is a 0.22 μm filter By adding fresh perfusate to the perfusate reservoir 104 while simultaneously directing a portion of the used perfusate into the waste container 145, the viscosity of the perfusate in circulation through the arterial and venous circuits 106, 108 can be dynamically adjusted.

In some implementations, based on detecting that the viscosity of the perfusate is outside a target range using the viscometer in the sensor block 124, the solenoid valve 146 is opened and the syringe pump manifold 167 is automatically controlled to introduce one or more chemical components into the perfusate reservoir 104 in order to adjust the viscosity of the perfusate flowing through the arterial and venous circuits 106, 108. For example, the syringe pump manifold 167 can be controlled to automatically introduce one or more crowding agents, thickening agents, polymeric agents, and/or droplet like agents in order to modify the mechanical properties of perfusate. In some implementations, the chemical component(s) that are introduced by the syringe pump manifold based on signals received from the viscometer are selected based on their ability to adjust the non-Newtonian properties of the perfusate to impart shear-thinning characteristics similar to the physiological shear-thinning characteristics present in blood. In some implementations, cone and plate rheometry is used to screen biocompatible polymers and lipid droplets with surfactant across physiological shear rates and to select blends of chemical components that exhibit a high shear velocity plateau of about 4 cP and thin to less than 2 cP in capillary-scale flow.

In some implementations, the viscosity of the perfusate detected by a viscometer in the sensor block 124 is provided as input to a trained machine learning model and, based on the viscosity data together with other sensor data provided to the machine learning model, the machine learning model generates control commands to automatically control the system 100 to adjust the viscosity of the perfusate to a particular viscosity target and, as a result, extend the potential length of perfusion.

The housing 126 of the sensor block 124 is fluidly coupled to the perfusate reservoir 104 along a sensor block outlet line 148 and an arterial sample line 150, and fresh perfusate flowing through the sensor block 124 is provided to the perfusate reservoir 104 along the sensor block outlet line 148 and/or the arterial sample line 150. A stopcock 153 is positioned along the arterial sample line 150, and can be used to sample the perfusate flowing out of the sensor block 124, for example, to conduct tests on the perfusate exiting the sensor block 124. In some implementations, the stopcock 153 is automatically controlled (e.g., by a servo-motor) to open a predetermined times throughout the duration of perfusion to enable automated sampling of perfusate in the arterial circuit 106.

Solenoid valves 149, 151 are positioned along the sensor block outlet line 148 and the arterial sample line 150, respectively, and are configured to control flow of fluid from the sensor block 124 to the perfusate reservoir 104. For example, the sensor block 124 includes a level sensor 125 that is configured to detect the level of fluid (e.g., fresh perfusate) inside the housing 126 of the sensor block 124, and the solenoid valve 149 along the sensor block outlet line 148 is controlled to open or close based on signals generated by the level sensor 125. In some implementations, the level sensor 125 is an optical level sensor, such as an infrared level sensor. For example, the solenoid valve 149 is controlled to remain in a closed state and prevent flow of fresh perfusate from the sensor block 124 into the perfusate reservoir 104 until the level sensor 125 generates a signal indicating that the fluid level inside the housing 126 of the sensor block 124 has exceeded a threshold fluid level. In response to the level sensor 125 generating a signal indicating that the fluid level inside the housing 126 of the sensor block 124 has exceeded a threshold fluid level, the solenoid valve 149 is controlled to open to allow perfusate to flow from the sensor block 124 along the sensor block outlet line 148 to the perfusate reservoir 104. Once the level sensor 125 detects that the fluid level inside the housing 126 of the sensor block 124 has fallen below the threshold level, the solenoid valve 149 is controlled to close, which prevents perfusate from flowing from the sensor block 124 to the perfusate reservoir 104 along the sensor block outlet line 148.

The sensor block 124 is fluidly coupled to the sensor line 1124 and a portion of the perfusate contained within the sensor block 124 flows through the sensor 1124 for testing of the perfusate by the sensor probes 128 along the sensor line 1124. For example, during perfusion, the solenoid valve 1120 along the arterial sensor line 1124 is open and the sensor pump 1122 along the sensor line 1124 is operated to draw perfusate from the sensor block 124, along the sensor line 1124, and back to the perfusate reservoir 104. As the perfusate passes through the sensor line 1124, sensor probes 128 along the sensor line 1124 collect data related to the perfusate. The sensor probes 128 are configured to measure certain characteristics of the perfusate flowing along the sensor 1124 including, but not limited to, pH, dissolved oxygen concentration, dissolved nitrogen concentration, dissolved carbon dioxide concentration, conductivity, redox potential, and temperature. Data generated by the sensor probes 128 while the solenoid valve 1120 along the arterial sensor line 1128 is open and fluid is flowing from the arterial circuit 106 through the sensor line 1124 can be automatically labelled as being related to perfusate in the arterial circuit 106.

As previously described, the oxygenator 116, the heat exchanger 118, and the gas mixer 120 are controlled based on the characteristics measured by the sensor probes 128 in order to optimize the temperature, O₂ level, and gas composition of the perfusate. In some implementations, an alpha-stat approach is used to control the concentration of gasses with the fresh perfusate and the pH of the fresh perfusate. In some implementations, a pH-stat approach is used to control the concentration of gasses with the fresh perfusate and the pH of the fresh perfusate.

Referring to FIG. 1, the perfusion system 100 includes a syringe pump manifold 167 that is configured to support one or more syringes and a stepper motor that is configured to control movement of a respective plunger of each of the syringes contained within the manifold 167. The syringe pump manifold 167 is fluidly coupled to the perfusate reservoir 104 along a drug input line 169.

In some implementations, a dialysis filtration system 109 and a syringe pump in the syringe pump manifold 167 that contains a buffer (e.g., sodium bicarbonate) can be automatically controlled to remove buffer from the perfusate or add buffer to the perfusate, respectively, based on the pH of the perfusate measured by the sensor probes 128 in order to maintain the pH of the perfusate within a physiological range. In some implementations, during calibration of the system 100, the data generated by the sensor probes 128 can be used to measure the change in pH of the perfusate following addition of a buffer (e.g., sodium bicarbonate) into the perfusate reservoir 104 and quantified response curves can be generated based on the pH data generated by the sensor probes 128 to validate and calibrate system responsiveness and buffering capacity of the syringe pump manifold 167.

In some implementations, the dialysis filtration system 109 and one or more syringe pumps in the syringe pump manifold 167 can be automatically controlled to remove electrolytes from the perfusate or add electrolytes to the perfusate, respectively, based on the electrolyte concentration of the perfusate measured by the sensor probes 128 in order to maintain the electrolyte concentration within a physiological range. In some implementations, during calibration of the system 100, the data generated by the sensor probes 128 can be used to measure the change in electrolyte concentration of the perfusate following addition of an electrolyte into the perfusate reservoir 104 and quantified response curves can be generated based on the data generated by the sensor probes 128 to validate and calibrate system responsiveness of the syringe pump manifold 167.

In some implementations, the syringe modules 167, 1167 are controlled by a PID controller. For example, the syringe manifold 167 containing a syringe with a buffer can be configured to maintain a threshold (e.g., 95% or greater) PID response efficiency to changes in the pH of the perfusate throughout a prolonged perfusion process (e.g., 2 weeks or longer of operation).

In some implementations, the data collected by the sensor probes 128 is provided to a trained machine learning model in real-time and, based on the data received from the sensor probes 128 together with other sensor data provided to the machine learning model, the machine learning model generates control commands to automatically control one or more mechanical components of the system 100 (e.g., the oxygenator 116, the heat exchanger 118, the gas mixer 120) to improve the quality of the perfusate and, as a result, extend the potential length of perfusion.

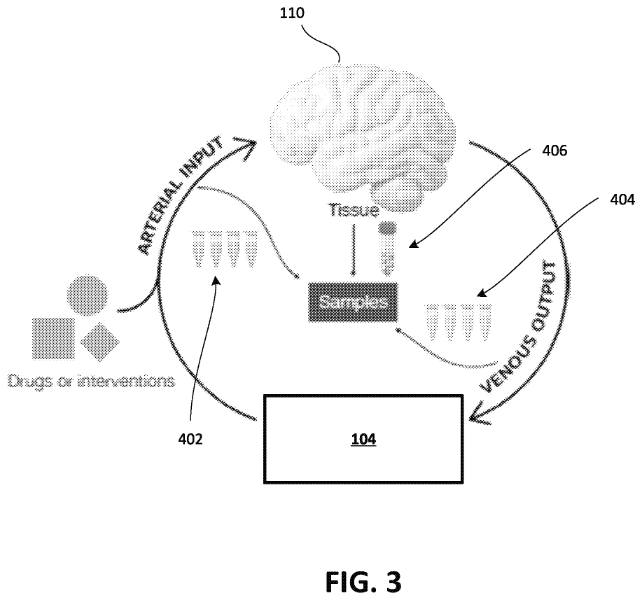

Referring to FIGS. 1 and 3, in addition to fluidly coupling the sensor block 124 to the perfusate reservoir 104, the arterial sample line 150 fluidly couples the sensor block 124 to an automatic sampler system 105. The automatic sampler system 105 includes a pump 184, a solenoid manifold 185, a sample collection line 186, and a purge line 187. The automatic sampler system 105 can be operated to automatically collect a sample of perfusate from the sensor block 124, for example, for testing one or more characteristics of the fresh perfusate contained inside the sensor block 124. A solenoid valve 171 is positioned along the arterial sample line 150 between the sensor block 124 and the automatic sampler system 105. In order to automatically collect a sample of perfusate from the arterial sample line 150, the solenoid valve 171 along the arterial sample line 150 between the sensor block 124 and the automatic sampler system 105 is opened, and the pump 184 of the automatic sampler system 105 is operated to draw perfusate out of the sensor block 124 along the arterial sample line 150, and the solenoid manifold 185 is controlled to direct the perfusate through the sample collection line 186 and into a sample collection vessel. The automatic sampler system 105 can be controlled to collect samples of fresh perfusate 302 from the sensor block 124 at predetermined intervals during the perfusion process. For example, in some implementations, the automatic sampler system 105 is configured to collect samples of fresh perfusate 302 from the sensor block 124 at the start of a perfusion process (“0 hour”), 6 hours after the start of a perfusion process, 12 hours after the start of a perfusion process, and every 24 hours after the start of a perfusion process for the length of the perfusion process (e.g., for two weeks or longer). Samples can alternatively be collected at any other desired frequency.

The purge line 187 of the automatic sampler system 105 is used to purge the contents of the arterial sample line 150 and the contents of a corresponding venous sample line 183 prior to collecting a fluid sample from the arterial sample line 150 or the venous sample line 183, respectively. For example, prior to sampling fluid from the arterial sample line 150, the solenoid valve 171 along the arterial sample line 150, a stopcock 1175 along the purge line 187, and a stopcock 1171 along the drain line 143 are automatically opened, the pump 184 and the solenoid manifold 185 of the automated sampler system 105 are controlled to draw the fluid contained inside the arterial sample line 150 through the purge line 187, through the drain line 143 to a waste container 145. The drain line 143 includes a one-way valve 1173 upstream of the stopcock 1171 fluidly coupling the purge line 187 to the drain line 143. The one-way valve 1173 prevents fluid flowing into the drain line 143 from the purge line 187 from flowing upstream away from the waste container 145. After a predetermined amount of time has elapsed, the solenoid manifold 185 is operated to direct the fluid flowing along the arterial sample line 150 through the sample collection line 186 and into a sample collection vessel. As a result of this purging process, the perfusate samples 402 collected by the automatic sampler system 105 from the arterial sample line 150 corresponds to fresh perfusate that has exited the sensor block 124 at the time the sample collection is performed, rather than perfusate that was present in the arterial sample line 150 prior to the time of sample collection.

Still referring to FIG. 1, a stopcock 155 is positioned along the organ line 144 downstream of the resistance valve 152. An operator of the system 100 can use the stopcock 155 to sample fluid from the organ line 144 downstream, of the resistance valve 152, for example, to test one of more properties of the perfusate being provided to the brain 110. In addition, the stopcock 155 can be operated in order to control the flow of air along the organ line 144 during initial set up and calibration of the system 100. For example, during initial calibration of the pressure sensor 141, pressurized air is provided to the organ line 144 along the air calibration line 163 through stopcock 155, and the stopcock 155 is closed to generate a stable air pressure along the organ line 144, and the pressure sensor 141 can be calibrated based on the measured pressure. For example, during calibration of the pressure sensor 141, an NIST certified manometer 1068 can be fluidly coupled to the air calibration line 163, the organ line 144 is pressurized with air provided along the air calibration line 163, and the pressure detected by the manometer 1068 is compared to the pressure detected by the pressure sensor 141 and is used to calibrate the pressure sensor 141.

A second stopcock 159 is positioned along the organ line 144 downstream of the pressure sensor 141 and the first stopcock 155. The stopcock 159 is configured to control fluid flow from the organ line 144 to a brain 110 inside the brain housing 102. For example, during perfusion of the brain 110, the stopcock 159 is automatically opened (e.g., using a servo-motor) to allow fluid within the organ line 144 flow to the brain 110 inside the brain housing 102. As will be described in further detail herein, the stopcock 159 is closed during calibration of the pressure sensor 141 and the flow sensor 142 to prevent fluid along the organ line 144 from flowing into the brain housing 102 during calibration of the pressure sensor 141 and flow sensor 142.

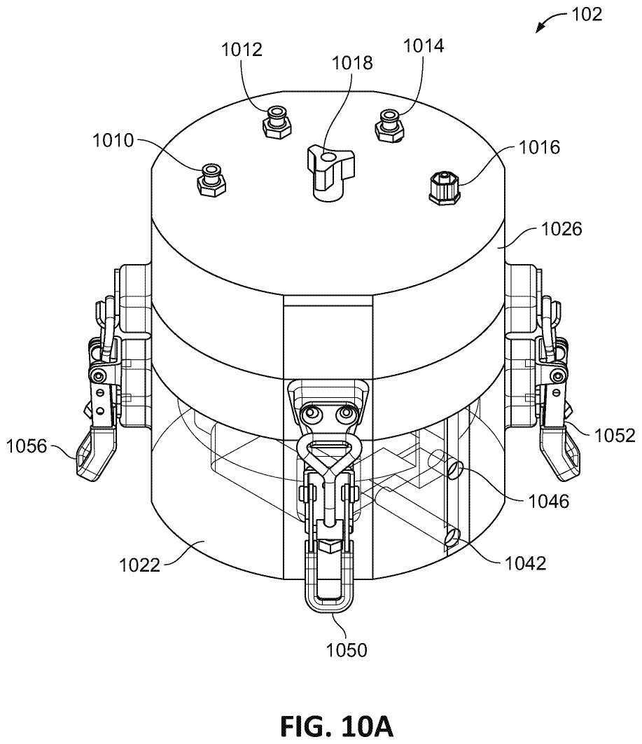

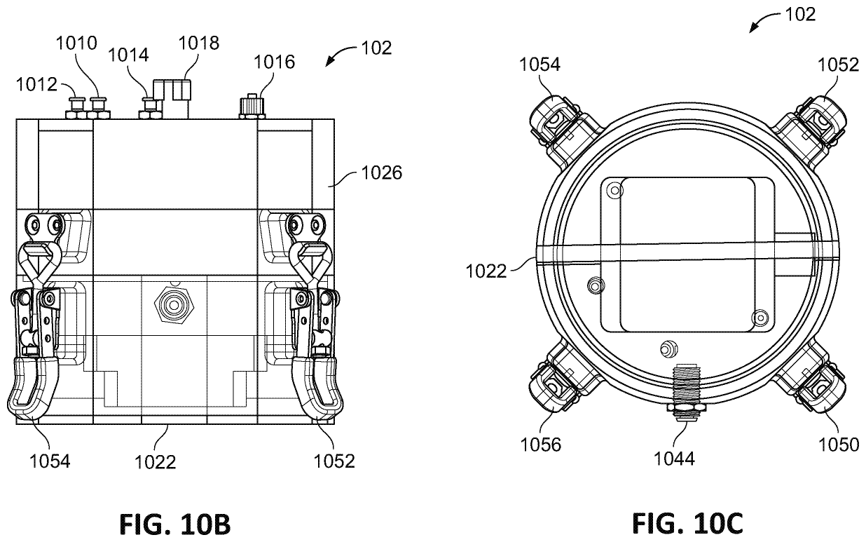

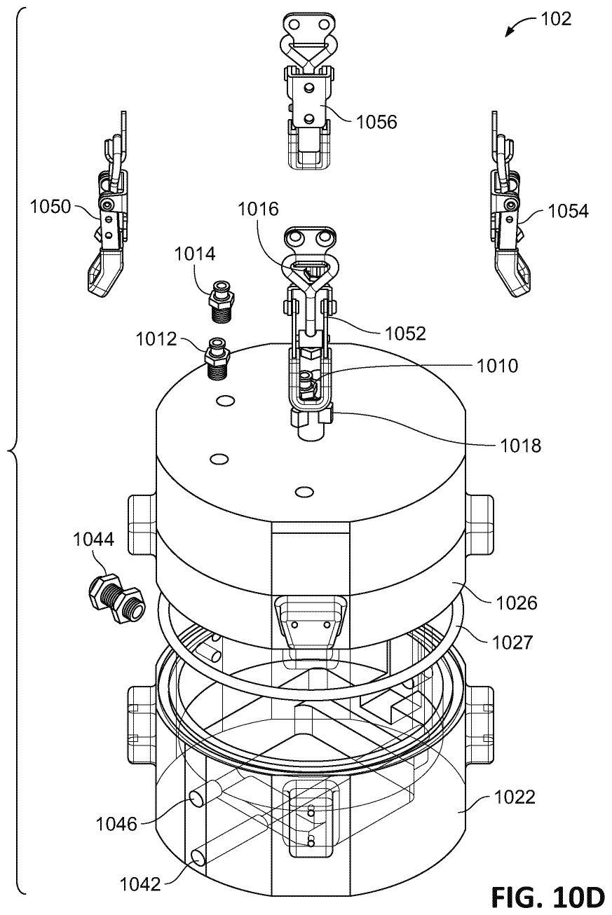

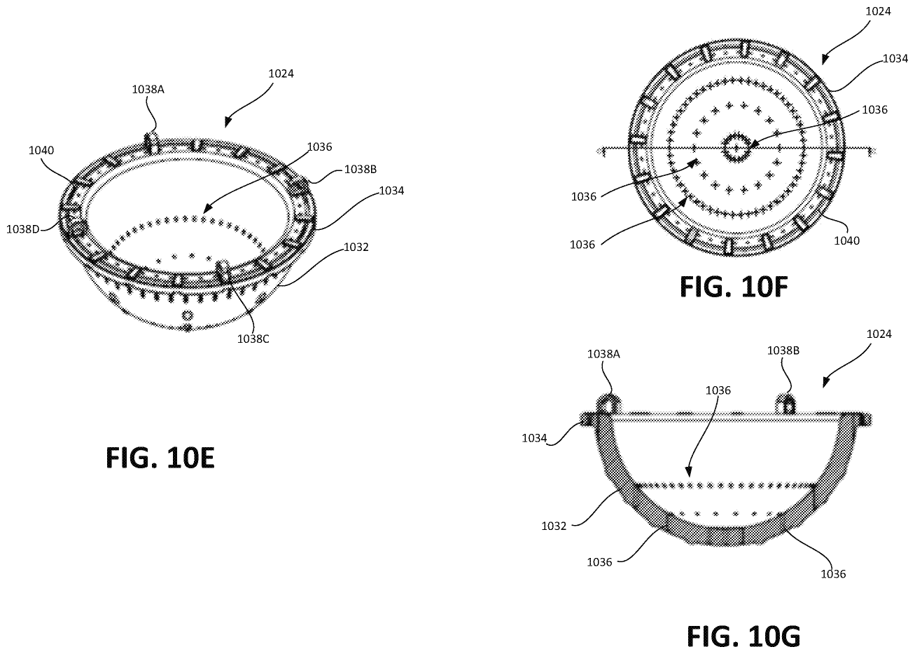

A brain housing shunt line 156 extends from the organ line 144 to the brain housing 102 and fluidly couples the organ line 144 to the interior of the brain housing 102. Referring to FIGS. 10A-10C, the brain housing 102 includes a fluid coupling 1046 that fluidly connects the shunt line 156 to the interior of the brain chamber 102. In some implementations, the coupling 1046 is a quick-turn tube coupling. The brain housing shunt line 156 is positioned downstream of the resistance valve 152 and upstream of the pressure sensor 141. A solenoid valve 158 is positioned along the brain housing shunt line 156 and can be operated to fluidly couple the organ line 144 to the brain housing 102 along the brain housing shunt line 156. In some implementations, the solenoid valve 158 along the brain shunt line 156 is opened to allow perfusate to flow from the organ line 144 to the brain housing 102 along the brain housing shunt line 156 during initial calibration of the pressure sensors 140, 141 and flow sensor 142 along the organ line 144. In addition, as will be described in further detail herein, the brain shunt line 156 can be used to perform a quality checking process on the perfusion system 100, including the calibration of the pressure sensor 141 and the flow sensor 142 along the organ line 144.

In some implementations, samples of pig vasculature are sutured to the carotid arteries and basilar artery of the human brain 110 at a first end and are fluidically connected to the organ line 144 (e.g., through suturing) at a second end. Perfusate flowing through the organ line 144 flows through the pig vasculature sutured to the brain 110, through the carotid and basilar arteries of the brain 110, and perfuses the brain 110.

The pressure and flow rate of the perfusate provided to the brain 110 are controlled to enable the perfusate entering the carotid arteries of the brain 110 to perfuse the entire vasculature of the brain 110, including the penetrating arterioles, pre-capillary arterioles, and capillaries of the brain 110.

The brain 110 is contained inside the brain housing 102 throughout the perfusion process. FIGS. 10A-C depict an example brain housing 102 and FIGS. 10E-10G depict an example hemispherical brain chamber 1024 of the brain housing 102. Referring to FIGS. 10A and 10D-10G, the brain housing 102 includes a basin 1022, a hemispherical brain chamber 1024, and an upper lid 1026.

The lid 1026 is configured to releasably couple to the basin 1022 of the brain housing 102 and can be separated from the basin 1022 to allow a brain 110 to be positioned inside the brain housing 102 on the hemispherical brain chamber 1024. In some implementations, the lid 1026 includes The brain housing 1102 also includes a three arm knob 1018 releasably coupled to the lid 1106 of the brain housing 1102. The knob 1018 serves as an ergonomic handle for opening the lid 1106 of the brain housing 1102. The brain housing 102 includes four latches 1050, 1052, 1054, 1056 that, when closed, hold the lid 1026 tightly onto the basin 1022. The brain housing 102 includes an O-ring 1027 positioned between the lid 1026 and the basin 1022. The O-ring 1027 is configured to form a pressure tight seal between the lid 1026 and the basin 1022, which enables the interior of the brain housing 102 to be pressured (e.g., to normal intracranial pressures). The lid 1026 of the brain housing includes a port fitting 1044 to fluidly couple the organ line to the brain 110 positioned in the brain housing 102. The port fitting 1044 is a push-to connect pressure tight port fitting. The pig vasculature sutured to the carotid arteries and basilar artery of the human brain 110 can then be fluidly coupled to the other side of the port fitting 1044 from the interior of the brain housing 102 (e.g., using a fluid line splitter device or other interface device).

The lid 1026 includes four Luer locks 1010, 1012, 1014, 1016 for fluidly coupling fluid lines, sensors and other devices to the brain housing 102. The lid 1026 of the brain housing includes a male Luer lock port 1016 configured to fluidly couple a brain housing pressure line 1131 to the interior of the brain housing 102. As will be described in detail herein, a filter 1165 is positioned along the brain housing pressure line 1131 and is configured to filter air being provided to the interior of the brain housing 102 from the brain housing pressure line 1131 to pressurize the brain housing 102. The lid 10126 includes three female Luer locks 1010, 1012, 1014. A first female Luer lock 101 fluidly couples a pressure sensor 1168 to the interior of the brain housing 102 and the pressure sensor 1168 monitors the pressure inside the brain housing 102. As will be described in further detail, the pressure sensor 1168 and the brain housing pressure line 1131 can be used to control the pressure inside the brain housing 102 to correspond to a normal intracranial pressure range. The other Luer locks 1012, 1014 can be used to fluidly couple other fluid lines and devices to the brain housing 102, such as an environment sensor 1522 and/or a microdialysis syringe pump 1506. The additional Luer locks 1012, 1014 can be capped off to prevent flow through the Luer locks 1012, 1014 when the Luer locks 1012, 1014 are not in use.

Referring to FIG. 10E-10G, the hemispherical brain chamber 1024 is configured to be positioned within the basin 1022 and is covered by the upper lid 1026 when the upper lid 1026 is attached to the basin 1022. During the perfusion process, the brain 110 is positioned on the hemispherical brain chamber 1024 with the carotid and basilar arteries of the brain 110 facing upwards toward the lid 1026.

Referring to FIGS. 10E-10G, the hemispherical brain chamber 1024 includes a chamber body 1032 that is configured to receive and support the brain 110 throughout a perfusion process. The chamber body 1032 is formed of a polymer material that provides a soft surface on which the brain 110 can be positioned and supported within the brain housing 102 throughout the perfusion process. The size, shape, and material of the chamber body 1032 are selected to maintain integrity of the brain 110 throughout the perfusion process. The chamber body 1032 defines a plurality of drain holes 1036 through the chamber body 1032. The drain holes 1036 allow perfusate flowing out of the veins of the brain 110 to flow through the hemispherical brain chamber 1024 into the basin 1022 such that the perfusate flowing out of the brain 110 is collected in the basin 1022.

As can be seen in FIGS. 10E-10G, the hemispherical brain chamber 1024 includes an upper lip 1034 extending from the chamber body 1032. When the hemispherical brain chamber 1024 is positioned within the basin 1022, the upper lip 1034 of the hemispherical brain chamber 1023 rests on an upper edge of the basin 1022 and allows the hemispherical brain chamber 1024 to be suspended within the basin 1022 without the contacting the bottom surface of the basin 1022 or blocking a level sensor 180 of the brain housing 102. The upper lip 1034 also defines a plurality of support slots 1040 configured to receive corresponding support structures of the basin 1022 that help support the weight of the brain 110 positioned on the hemispherical brain chamber 1024.

Referring to FIGS. 10E and 10G, the hemispherical brain chamber 1024 includes loops 1038A-1038D that are coupled to the upper lip 1034 and that can be used to facilitate removal of the hemispherical brain chamber 1024 from the basin 1022. For example, an operator of the system 100 can grasp one or more of the loops 1038A-1038D with her fingers to lift the hemispherical brain chamber 1024 out of the basin 1022.

The brain housing 102 is configured to maintain the brain 110 under normothermic conditions. For example, when the basin 1022 and lid 1026 of the brain housing 102 are securely coupled together, a warm, humid environment is maintained within the brain housing 102. In some implementations, the brain housing 102 includes temperature and humidity controls configured maintain a particular temperature and humidity level to prevent drying or other injury to the brain 110. As depicted in FIG. 1, the brain housing 102 includes an environment sensor 1522 that detects one or more conditions of the interior of the brain housing 102 including, but not limited to, the temperature of the brain housing 102, the level of humidity of the brain housing 102, and the differential pressure inside the brain housing 102. Based on the signals generated by the environment sensor 1522, one or more components of the perfusion system 100 can be automatically to optimize the environment inside the brain housing 102. For example, based on the temperature and humidity information generated by the environment sensor 1522, the heat exchanger 118 can be automatically controlled to adjust the temperature of the perfusate flowing through the system 100. In addition, based on the differential pressure inside the brain housing 102 detected by the brain housing 102, the pressure regulator 1166 on the brain housing pressure line 1131 can be automatically controlled to adjust the pressure inside the brain housing 102. By dynamically adjusting the environment inside the brain housing 102, the viability of the tissued of the brain 110 can be maintained and the length of perfusion can potentially be extended (e.g., to two weeks or longer of perfusion).

After the perfusate flows through the capillaries of the brain 110, the perfusate flows out of the brain 110 via veins, through the drain holes 1036 in the hemispherical brain chamber 1024, and into the basin 1022 of the brain housing 102. The basin 1022 of the brain housing 102 collects and contains the perfusate that flows out of the brain 110 (“used perfusate”).

As depicted in FIG. 1, the perfusion system 100 includes a camera 1520 coupled to the brain housing 102. The camera 1520 is configured to generate high-resolution image data (e.g., photographs and/or video data) throughout the perfusion process that depicts the brain 110 contained inside the brain housing 102. In some implementations, the camera 1520 is a Nicla Vision sensor manufactured by Arduino®.

The image data generated by the camera 1520 can be processed by the system 100 to predict the health of the brain 110. For example, the image data generated by the camera 1520 depicting the surface of the brain 110 can be provided to a trained machine learning model to automatically detect markers of viability of the tissue of the brain 110 including, but not limited to, the color of the brain 110, the size of the brain 110, and/or the shape of the brain 110 and, based on these detections, determine the health of the brain 110 in real-time. For example, the perfusate provided to the brain 110 via the perfusion system 100 can include a dye and the color of the brain 110 can be monitored in real time based on the image data generated by the camera 1520 to determine how efficiently the perfusate is being flushed out of the brain 110.

In addition, based on detecting changes in the size and shape of the brain 110 based on processing the image data generated by the camera 1520, the machine learning model can predict the amount of swelling in the brain 110 and can automatically control one or more components of the perfusion system 100 to reduce brain swelling, which can potentially extend the length of perfusion. For example, based on detecting an increased rate of swelling of the brain 110, the machine learning model can generate command signals to control (i) the heat exchanger to adjust the temperature of the perfusate, (ii) the pressure regulator 1166 on the brain housing pressure line 1131 to adjust the pressure inside the brain housing 102, (iii) one or more of the syringe pumps in the syringe pump manifolds 167, 1167, the stopcock 1149, the solenoid valve 149 to add one or more substances to the perfusate in the perfusate reservoir 104, (iv) the supplement pump 1906 to add fresh perfusate from the supplementary fresh perfusate reservoir 1903 to the reservoir 104, and/or (v) the dialysis filtration system 109 to remove one or more substances from the perfusate.

In some implementations, in addition to generating image data of the surface of the brain 110, the camera 1520 is configured to generate image data depicting the perfusate inside the brain housing 102, and the image data depicting the perfusate inside the brain housing 102 can be provided to the trained machine learning model to determine a state of the brain 110 and the perfusate. For example, the trained machine learning model can process the image data depicting the perfusate inside the brain housing 102 to automatically detect changes in the color of the perfusate inside the brain housing 102 in real-time and, based on the detected changes in the perfusate color, can determine control one or more of the syringe pumps 167, 1167, the dialysis filtration system 109, and/or the supplement pump 1906 to adjust the composition of the perfusate flowing through the system 100 in order to improve viability of the brain tissue and potentially extend the length of the perfusion.

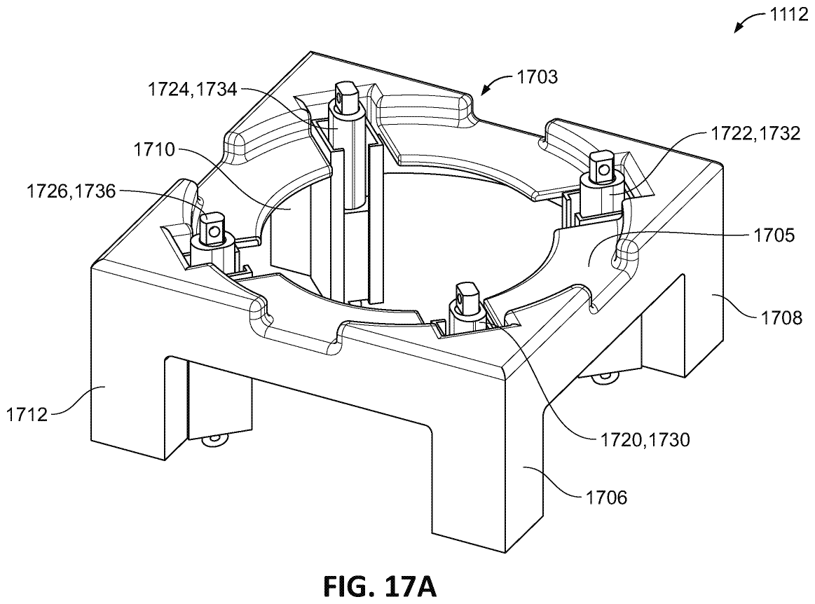

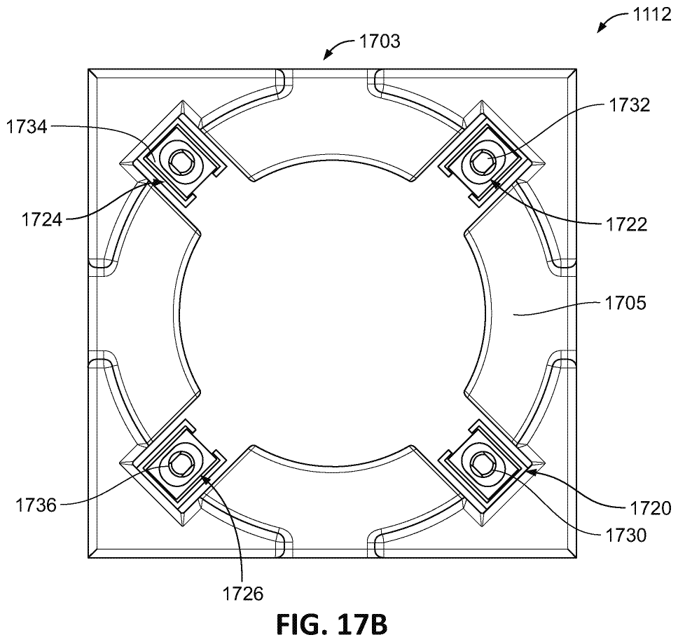

The brain housing 102 is positioned on a brain housing stand 1112 that supports and dynamically repositions the brain housing 102 throughout the perfusion process. FIGS. 17A and 17B depict an example brain housing stand 1112. As can be seen in FIG. 17A, the brain housing stand 1112 includes a frame 1703 with a base 1705 and four legs, 1706, 1708, 1710, 1712 extending from the base 1705. The brain housing stand 1112 also includes four linear actuators 1720, 1722, 1724, 1726 that positioned within and extend through the respective four legs four legs, 1706, 1708, 1710, 1712 of the frame 1703. The rod 1730, 1732, 1734, 1736 of each of the four linear actuators 1720, 1722, 1724, 1726 extends above surface of the base 1705. The brain housing 102 containing the brain 110 is configured to be position on and supported by the base 1705 of the frame 1703 throughout perfusion.

When the brain housing 102 is positioned on the brain housing stand 1112, rod 1730, 1732, 1734, 1736 of each of the four linear actuators 1720, 1722, 1724, 1726 is in contact with the brain housing 102. Throughout perfusion of the brain 110, the system 100 controls the rods 1730, 1732, 1734, 1736 of the linear actuators 1720, 1722, 1724, 1726 to extend and retract in sequence (e.g., one at a time) at predetermined times throughout the perfusion process. When the rod 1730 of one of the linear actuators 1720, is extended while the rods 1732, 1734, 1736 of the other three linear actuators 1722, 1724, 1726 remain retracted, the portion of the brain housing 102 positioned over the extended linear actuator 1720 and in contact with the extended rod 1730 will be raised, causing the brain housing 102 to be tilted at an angle. After a predetermined amount of time has elapsed, the rod 1730 of the first linear actuator 1720 is retracted, and the rod 1732, 1734, 1736 of one of the other three linear actuators 1722, 1724, 1726 is extend. The rods 1730, 1732, 1734, 1736 of each of the linear actuators 1720, 1722, 1724, 1726 are extended and retracted in sequence repeatedly throughout perfusion of the brain 110 to continuously reposition and tilt the brain housing 102. By repeatedly repositioning and tilting the brain housing 102 throughout the perfusion process, the localized pressure on the brain 110 caused by contact between the brain 110 and the brain housing 102 is reduced, which results in a reduction in localized pressure-induced ischemia of the tissue of the brain 110 compared to localized pressure-induced ischemia of the tissue of the brain 110 that occurs with static positioning of the brain 110 during perfusion due to gravitational and contact-induced stress on the tissue. In addition, by repeatedly repositioning and tilting the brain housing 102 throughout the perfusion process using the brain housing stand 1112, swelling of the brain 110 during perfusion can be reduced by approximately 5% (or more) compared to static positioning of the brain 110 during perfusion. As a result, use of the brain housing stand 1112 can potentially extend the length of perfusion of the brain compared to perfusion performed with a stationary brain housing.

The brain housing 102 and the brain housing stand 1112 are positioned on a brain housing scale 174 that measures the weight of the brain 110 and the contents inside the housing (e.g., the brain 110 and used perfusate) throughout the perfusion process. Based on the weight measurements generated by the brain housing scale 174, the amount of swelling of the brain 110 due to fluid retention can be estimated in real-time throughout the perfusion process. In addition to monitoring swelling, the brain housing scale 174 can be used also to calculate and validate the flow measurements generated by the flow sensor 142 along the organ line 144. For example, the amount of perfusate provided to the brain housing 102 during a particular period of time as determined by the weight measured by the brain housing scale 174 can be compared to the amount of perfusate flow measured by the flow sensor 142 during the particular time period in order to validate the measurements of the flow sensor 142. In addition, the brain housing scale 174 can be used to validate the time at which samples of perfusate is collected from the brain housing 102 by the automatic sampler system 105.

A housing outlet line 176 extends from the brain housing 102 and fluidly couples the brain housing 102 to the perfusate reservoir 104. A solenoid valve 178 is positioned along the housing outlet line 176 to control the flow of used perfusate from the brain housing 102 into the perfusate reservoir 104. For example, when the solenoid valve 178 is open, used perfusate collected in the brain housing 102 flows along the housing outlet line 176 into the perfusate reservoir 104.

Referring to FIG. 10C, the brain housing 102 includes a coupling 1042 connected to the side of the basin 1022. The coupling 1042 fluidly connects the interior of the basin 1022 of the brain chamber 102 to the housing outlet line 176. In some implementations, the coupling 1042 is a quick-turn tube coupling. In some implementations, the brain housing 102 includes a second coupling that can be coupled to the outlet line 176 or to another outlet line in order to increase the rate of drainage of fluid out of the brain housing 102 to the perfusate reservoir 104.

The brain housing 102 is fluidly coupled to the air supply 160 along a brain housing pressure line 1131 that fluidly couples the air supply line 161 to the brain housing 102 in order to pressurize the interior of the brain housing 102. The lid 1026 includes a Luer lock port 1012 configured to fluidly couple the interior of the brain housing 102 to the pressure line 1331. An electronic pressure regulator 1166 is positioned along the brain housing pressure line 1131 and a and a pressure sensor 1168 is fluidly coupled to the interior of the brain housing 102 via the Luer lock port 1010 on the brain housing lid 1026. Together, the electronic pressure regulator 1166 and the pressure sensor 1168 control the pressure inside the housing 102. For example, the pressure regulator 1166 along the brain housing pressure line 1161 is an electronic pressure regulator that has tighter tolerances compared to the first pressure regulator 162 and is configured to provide a pressurized air stream to the brain housing 102. The pressure of the air stream generated by the electronic pressure regulator 1166 is controlled based on pressure signals received from the pressure sensor 1168 of the brain housing pressure line 1161, which measures the pressure along the brain housing pressure line 1161 and within the brain housing 102. A stopcock 1169 fluidly couples the brain housing pressure line 1131 to the interior of the brain housing 102 and can be controlled to automatically open (e.g., using a servo-motor) to fluidly couple the brain housing pressure line 1131 to the interior of the brain housing 102.

In some implementations, the pressure regulator 1166 is controlled, based on signals received from the pressure sensor 1168, to provide a pressurized air stream to the brain housing that pressurizes the interior of the brain housing in a range that corresponds to physiological intracranial pressure (e.g., in a range of 5 mmHg to 15 mmHg). When the brain housing 102 is pressurized by the air pressure system 138, the overall cerebral perfusion pressure provided to a brain 110 in the brain housing 102 being perfused by the perfusion system 100 is equal to the difference between the average pressure of perfusate flowing into the brain 110 along the organ line (i.e., mean arterial pressure) and the intracranial pressure supplied to the brain housing 102 along the brain housing pressure line 1161.

Pressurizing the interior of the brain housing 102 to a pressure range similar to physiological intracranial pressure improves tissue integrity and viability of the brain contained within the brain housing 102. In addition, pressurizing the interior of the brain housing 102 to a pressure range similar to physiological intracranial pressure improves the histological and metabolic viability of the brain 110 contained within the brain housing 102. As a result, pressurizing the interior of the brain housing 102 to a pressure range similar to physiological intracranial pressure enables the brain 110 to be perfused by the perfusion system 100 for extended periods (e.g., two weeks or longer) while maintaining normal cellular activity of the brain 110.

A filter 1165 is positioned along the brain housing pressure line 1131 in order to filter out and prevent any particulates in the pressurized air stream flowing along the brain housing pressure line 1131 from entering the brain housing 102. In some implementations, the filter 1165 is a 0.22 μm filter.

The brain housing 102 includes a level sensor 180 to detect the level of fluid (e.g., used perfusate) inside the basin 1022 of the brain housing 102, and the solenoid valve 178 along the housing outlet line 176 is controlled to open or close based on signals generated by the level sensor 180. As depicted in FIG. 10C, the level sensor 180 is coupled to the basin 1022 of the brain chamber 102. In some implementations, the level sensor 180 is coupled to the basin 1022 at the same or similar height along the basin 1022 as the fluid coupling 1046 that fluidly connects the shunt line 156 to the interior of the brain chamber 102. In some implementations, the level sensor 180 is an optical level sensor, such as an infrared level sensor. For example, the solenoid valve 178 is controlled to remain in a closed state and prevent flow of used perfusate from the brain housing 102 into the perfusate reservoir 104 until the level sensor 180 generates a signal indicating that the fluid level inside the brain housing 102 has exceeded a threshold fluid level. In response to the level sensor 180 generating a signal indicating that the fluid level inside the brain housing 102 has exceeded a threshold fluid level, the solenoid valve 178 and the solenoid valve 1121 along the venous sensor line 1129 are opened and the sensor pump 1122 is operated to pump used perfusate from the brain housing 102 along the housing outlet line 176 and along the sensor line 1124 to the perfusate reservoir 104. Once the level sensor 180 detects that the fluid level inside the brain housing 102 has fallen below the threshold level, the solenoid valves 178, 1121 are closed, which prevents fluid from flowing from the brain housing 102 to the perfusate reservoir 104.

Four stopcocks 181, 182, 1181, 1183 are positioned along the housing outlet line 176 between the brain housing 102 and the perfusate reservoir 104. An operator of the system 100 can use the stopcocks 181, 182, 1181, 1183 to sample the used perfusate flowing out of the brain housing 102, for example, to test one of more properties of the used perfusate discharged from the brain 110. In some implementations, the stopcocks 181, 182, 1181, 1183 are automatically operated (e.g., using a servo-motor) to sample the used perfusate flowing out of the brain housing 102 at predetermined intervals throughout the perfusion process.

The stopcock 181 along the housing outlet line 176 fluidly couples the housing outlet line 176 to a venous sample line 183, which fluidly couples the brain housing 102 to the automatic sampler system 105. The automatic sampler system 105 can be operated to automatically collect a sample of used perfusate from the brain housing 102, for example, to test one or more characteristics of the used perfusate. A solenoid valve 173 is positioned along the venous sample line 183 between the brain housing 102 and the automatic sampler system 105. In order to automatically collect a sample of perfusate from the venous sample line 183, the stopcock 181 is automatically controlled (e.g., using a servo motor) to fluidly couple the housing outlet line 176 to the venous sample line 183, the solenoid valve 173 along the venous sample line 183 is opened, and the pump 184 of the automatic sampler system 105 is operated to draw used perfusate out of the brain housing 102 along the venous sample line 183, and the solenoid manifold 185 is controlled to direct the used perfusate through the sample collection line 186 and into a sample collection vessel. A one-way valve 1151 is positioned along the venous sample line 183 downstream of the stopcock 181 to prevent fluid flowing along the venous sample line 183 from flowing backwards into the housing outlet line 176. Referring to FIGS. 1 and 3, the automatic sampler system 105 can be controlled to collect samples of used perfusate 304 from the brain housing 102 at predetermined intervals during the perfusion process. For example, in some implementations, the automatic sampler system 105 is configured to collect samples of used perfusate 304 from the brain housing 102 at the start of a perfusion process (“0 hour”), 6 hours after the start of a perfusion process, 12 hours after the start of a perfusion process, and every 24 hours after the start of a perfusion process for the length of the perfusion process (e.g., for two weeks or longer). Samples can alternatively be collected at any other desired frequency.

The automatic sampler system 105 purges the contents of the venous sample line 183 prior to collecting a fluid sample from the venous sample line 183. For example, prior to sampling fluid from the venous sample line 183, the solenoid valve 173 along the venous sample line 183 is opened, the pump 184 and the solenoid manifold 185 of the automated sampler system 105 are controlled to draw the fluid contained inside the venous sample line 183 through the purge line 187 to a drain or waste container. After a predetermined amount of time has elapsed, the solenoid manifold 185 is operated to direct the fluid flowing along the venous sample line 183 through the sample collection line 186 and into a sample collection vessel. As a result of this purging process, the samples 404 collected by the automatic sampler system 105 from the venous sample line 183 correspond to used perfusate that exited the brain housing 102 at the time of the sample collection, rather than used perfusate that was present in the venous sample line 183 prior to the time of sample collection.

The stopcock 1183 along the along the housing outlet line 176 fluidly couples the housing outlet line 176 to a venous sensor line 1129. During perfusion, the solenoid valve 1120 along the arterial sensor line 1124 can be closed open, the solenoid valve 1121 along the venous sensor line 1129 can be opened, and the sensor pump 1122 along the sensor line 1124 can be operated to draw perfusate from the housing outlet line 176, along the venous sensor line 1129 and the sensor line 1124, and back to the perfusate reservoir 104. As the used perfusate exiting the brain housing 102 passes through the sensor line 1124, sensor probes 128 along the sensor line 1124 collect data related to the used perfusate exiting the brain housing 102. The sensor probes 128 are configured to measure certain characteristics of the perfusate flowing along the sensor 1124 including, but not limited to, pH, dissolved oxygen concentration, dissolved nitrogen concentration, dissolved carbon dioxide concentration, conductivity, redox potential, and temperature. Data generated by the sensor probes 128 while the solenoid valve 1120 along the arterial sensor line 1128 is closed, the solenoid valve 1121 along the venous sensor line 1129 is open, and fluid is flowing from the venous circuit 108 through the sensor line 1124 can be automatically labelled as being related to perfusate in the venous circuit 108. In some implementations, samples of perfusate are withdrawn from the brain housing 102 and passed through the sensor line 1124 in response to a level sensor 180 of the brain housing 102 detecting that the level of perfusate inside the brain housing 102 has exceeded a threshold level.

As previously described, the oxygenator 116, the heat exchanger 118, and the gas mixer 120 are controlled based on the characteristics measured by the sensor probes 128 in order to optimize the temperature, O₂ level, and gas composition of the perfusate. In some implementations, the data collected by the sensor probes 128 while used perfusate from the venous circuit 108 is flowing through the sensor line 1124 is provided to a trained machine learning model in real-time and, based on the data received from the sensor probes 128 together with other sensor data provided to the machine learning model, the machine learning model generates control commands to automatically control one or more mechanical components of the system 100 (e.g., the oxygenator 116, the heat exchanger 118, the gas mixer 120) to improve the quality of the perfusate and, as a result, extend the potential length of perfusion.

The perfusion system 100 also includes a microdialysis system 1502 that is configured to extract interstitial fluid from the brain's inter-vascular space via microdialysis. The microdialysis system 1502 includes a microdialysis membrane 1504, a microdialysis syringe pump 1506, a buffer solution line 1507, a collection line 1508, and a microdialysis collection receptacle 1510. The microdialysis membrane 1504 is implanted into the tissue of the brain 110 in the brain housing 102. The microdialysis syringe pump 1506 pumps a buffer solution to the microdialysis membrane 1504 along the buffer solution line 1507. As the buffer solution flow through the microdialysis membrane 1504 into the tissue of the brain 110, the buffer solution reacts with the interstitial fluid of the brain and causes a portion of the interstitial fluid containing to flow through the membrane (e.g., via passive diffusion) along the collection line 1508 and into the microdialysis collection receptacle 1510.

The sample of interstitial fluid collected from the brain 110 in the microdialysis collection receptacle 1510 can be analyzed using one or more testing methodologies (e.g., mass spectrometry, high-performance liquid chromatography (HPLC), etc.) in order to detect the levels of metabolic and pharmacokinetic markers. For example, downstream analysis of the interstitial fluid collected from the brain 110 by the microdialysis system 1502 can be used to detect key tissue metabolites in the brain including, but not limited to, lactate, glucose, and neurotransmitters. The data generated based on analyzing the interstitial fluid collected from the brain 110 by the microdialysis system 1502 can be provided to a trained machine learning model to detect the health of the brain 110 and, based on the detected brain health, control the system 100 to dynamically adjust the chemical composition of the perfusate in order to improve brain health and extend the length of perfusion.

Still referring to FIG. 1, the perfusate reservoir 104 includes a reservoir housing 103 that is configured to receive fresh perfusate from the sensor block 124 along the sensor block outlet line 148 and along the arterial sample line 150, and to receive used perfusate from the brain housing 102 along the housing outlet line 176. The reservoir housing 103 is a watertight container that can be formed of any suitable material including, but not limited to, plastic, metal, or glass. The perfusate reservoir 104 includes a filter bag 101 positioned within the reservoir housing 103 to filter the fluid flowing into the perfusate reservoir 104. In some implementations, the filter bag 101 is formed of a 0.22 μm filter.

In addition to the filter bag 101 positioned inside the reservoir housing 103, the perfusion system 100 includes an in-line mechanical filtration system 1001 that includes a peristaltic filtration pump 1002, a filter manifold 1114 fluidly coupled to the filtration pump 1002 along a filtration line 1006, and a pressure sensor 1008 positioned along the filtration line 1006 between the filtration pump 1002 and the filter manifold 1114 and a flow sensor 1107 positioned along the filtration line 1006 downstream of the filtration manifold 1114. The filter manifold includes three 0.2/0.1 μm scrubbing filters 1003, 1004, 1005 that are connected the filtration line 1006 in parallel.

In order to further filter the perfusate contained in the perfusate reservoir 104 prior to providing the perfusate to the brain 110, the filtration pump 1002 can be operated to draw fluid out of the perfusate reservoir 104 along the filtration line 1006, through one of the filters 1003, 1004, 1005 in the filtration module 1114, and back to the perfusate reservoir 104. The filtration pump 1002 is communicably coupled to the pressure sensor 1008, and the rate of the filtration pump 1002 is controlled to maintain a predetermined range of pressure along the filtration line 1006, as detected by the pressure sensor 1008. In some implementations, the filtration pump 1002 is controlled based on signals from the pressure sensor 1008 to maintain the pressure along the filtration line 1006 within a range of 50 mmHg and 150 mmHg. In some implementations, the filtration pump 1002 is controlled to generate a particular pressure along the filtration line 1006, as measured by pressure sensor 1008, based on the condition of the perfusate and the level of residue in the filter 1003, 1004, 1005 being operated to filter the perfusate.

The filtration pump 1002 is also communicably coupled to the flow sensor 1107 positioned along the filtration line 1006 downstream of the filtration manifold 1114 and the rate of the filtration pump 1002 is controlled to maintain the flow rate along the filtration line 1006 within a target range. In some implementations, the filtration pump 1002 is controlled to maintain the flow rate along the filtration line 1006, as detected by the flow sensor 1107, within a target range while simultaneously controlling the pressure along the filtration line 1006, as detected by the pressure sensor 1008, to remain below a threshold pressure.

As the perfusate flows through one of the filters 1003, 1004, 1005 in the filtration module 1114, additional particulates and other potential contaminants are removed from the perfusate before returning the perfusate to the perfusate reservoir 104 and flowing the perfusate to the brain 110. The in-line mechanical filtration system 1001 can be operated throughout the process of perfusing the brain 110 to further filter the perfusate any bacteria or contaminates. The filtration module 1114 includes three filters 1003, 1004, 1005 that are fluidly coupled to the filtration line 1006 in parallel to provide redundancy in case one or more of the filters becomes clogged or otherwise starts to function sub optimally during the perfusion process. For example, at the beginning of a perfusion process, the stopcocks 1109, 1111 upstream of a second filter 1004 and a third filter 1005, respectively, and the stopcocks 1113, 1115 downstream of the second filter 1004 and the third filter 1005, respectively, are closed. As a result, the perfusate fluid flowing along the filtration line 1006 is pumped through the first filter 1003 without passing through the second or third filters 1004, 1005. Throughout perfusion process, signals generated by the pressure sensor 1008 and flow sensor 1107 are processed (e.g., by a machine learning module) to detect whether the one or more filters of the filtration manifold 1114 that are in use are becoming clogged or otherwise operating sub optimally. For example, at the beginning of perfusion, only the first filter 1003 is in use and the first filter 1003 can be determined as being clogged in response to determining that (i) the pressure along the filtration line 1006, as determined based on signals generated by the pressure sensor 1008, is above a threshold pressure and/or (ii) the flow rate of perfusate along the filtration line 1006 downstream of the filtration module 1114, as determined based on signals generated by the flow sensor 1107, is below a threshold flow rate. In response to determining that the first filter 1003 is becoming clogged or otherwise operating sub optimally, the stopcocks 1109, 1113 upstream and downstream of the second filter 1004 are opened to allow perfusate flowing along the filtration line 1006 to pass through the second filter 1004. Further, the system can similarly detect, when the second filter 1004 is in use, that the second filter 1004 is becoming clogged or otherwise operating sub optimally based on signals generated by the pressure sensor 1008 and/or the flow sensor 1107. In response to detecting that both the first filter 1003 and the second filter 1004 becoming clogged or otherwise operating sub optimally, the stopcocks 1111, 1115 upstream and downstream of the third filter 1005 are opened to allow perfusate flowing along the filtration line 1006 to pass through the third filter 1005. The system 100 can similarly detect that the third filter 1005 is becoming clogged or otherwise operating sub optimally based on signals generated by the pressure sensor 1008 and/or the flow sensor 1107. In response to determining that all three filters 1003, 1004, 1005 of the filtration manifold 1114 are clogged or otherwise operating sub optimally, the system 100 can generate an alarm and/or automatically shut down the in-line mechanical filtration system 1001 (e.g., by automatically stopping the pumping of the filter pump 1002). By providing a filter manifold 1114 with three filters 1003, 1004, 1005 that can be independently controlled, the in-line mechanical filtration system 1001 is capable of filtering perfusate flowing through the system 100 throughout extended periods of perfusion (e.g., 2 weeks or longer).

The filtration manifold 1114 is configured to maintain bacterial levels in the perfusate below target levels throughout the perfusion process. As discussed above, in response to determining, based on the signals generated by the pressure sensor 1008 and/or the flow sensor 1107, that one or more of the filters 1003, 1004, 1005 is performing sub optimally, the manifold 1114 is controlled to flow the perfusate through another one of the filters 1003, 1004, 1005 in the manifold 1114.

In some implementations, the filters 1003, 1004, 1005 retain 85% or more of their original removal efficiency for target molecules (e.g., metabolic waste, inflammatory compounds, etc.) throughout a testing period of 24 hours or longer. In some implementations, the filters 1003, 1004, 1005 exhibit a pressure drop within an acceptable ranges (e.g., less than 20% increase in pressure drop from the baseline pressure drop of the filter) throughout a testing period of 24 hours or longer. In addition, the filters 1003, 1004, 1005 do not exhibit structural failure or bypass flow during extended use (e.g., during a perfusion period of two weeks or longer).

In some implementations, prior to perfusion using the system 100, the pressure sensor 1008 is calibrated using a manometer 1068 (e.g., a NIST-certified manometer).

The perfusion system 100 also includes a dialysis filtration system 109 that is configured to further filter the perfusate flowing through the system 100 and remove metabolic waste products or other unwanted substances from the perfusate. The dialysis filtration system 109 includes a dialysis pump 190, a dialyzer manifold 191, a dialyzer inlet line 192 coupled to the dialyzer manifold 191, and a dialyzer outlet line 193 coupled to the dialyzer manifold 191. The dialyzer manifold 191 three dialyzers 1191, 1192, 1193 that are fluidly coupled to the dialyzer inlet line 192 in parallel. Each of the dialyzers 1191, 1192 in the dialyzer manifold 191 includes a membrane configured filter waste particles out of the perfusate. In some implementations, the dialysis membrane is configured to maintain a rate of solute clearance (e.g., urea and/or creatinine) of 90% of more of the initial solute clearance rate after 24 hours or longer of perfusion. In addition, the dialysis membrane is configured to maintain physical integrity (e.g., no tears, channeling, or clogging) and maintain a flow-through rate and pressure drop within design parameters throughout a perfusion process lasting 24 hours or longer.

In some implementations, the dialysis filtration system 109 is configured to filter substances including, but not limited to, urea, creatinine, and lactate, to a dialysis solution receptacle 117. As will be discussed in further detail herein, the substances filtered out of the perfusate by the dialysis filtration system 109 can be detected by the Raman spectroscopy system 109 and sensor module 2107 positioned downstream of the dialyzer manifold 191 and upstream of the dialysis solution receptacle 117.

The dialysis pump 190 is a peristaltic pump that is controlled throughout the perfusion process to draw perfusate from the perfusate reservoir 104 along the dialyzer inlet line 192 and into one or more of the dialyzers 1191, 1192, 1193 of the dialyzer manifold 191 at a controlled pressure. As the perfusate flows through one or more of the dialyzers 1191, 1192, 1193 of the dialyzer manifold 191, waste particles within the perfusate are filtered out of the perfusate by the membrane of each of the dialyzers 1191, 1192, 1193 being operated to receive perfusate and the filtered perfusate exits the dialyzer manifold 191 along the dialyzer outlet line 193.

Stopcocks 1195, 1197, 1199 are positioned along the dialyzer outlet line 193 downstream of each of the respective dialyzer 1191, 1192, 1193. The stopcocks 1195, 1197, 1199 can be used to fluidly couple the respective dialyzers 1191, 1192, 1193 to the dialyzer outlet line 193 and to sample perfusate fluid flowing out of the dialyzer manifold 191 to test one of more properties of the perfusate being provided to the perfusate reservoir 104, for example, to test the effectiveness of the dialysis filtration system 109.

The dialysis filtration system 109 includes a pressure sensor 194 positioned upstream of the dialyzer 191 and a flow sensor 195 positioned downstream of the dialyzer 191, and the rate of the dialysis pump 190 is controlled based on pressure signals generated by pressure sensor 194 195 in order to control the pressure within the dialyzer 191 to provide efficient filtration across the membrane of the dialyzer 191. For example, the pressure upstream of the dialyzer 191 can be measured by the pressure sensor 194 and the pump rate of the dialysis pump 190 can be controlled in order to maintain a pressure along the dialyzer inlet line 192 within a predetermined range that is sufficient to enable efficient waste removal and nutrient exchange within the dialyzer manifold 191. The dialysis pump 190 is also communicably coupled to the flow sensor 195 positioned along the dialyzer outlet line 193 downstream of the dialyzer manifold 191 and the rate of the dialysis pump 190 is controlled to maintain the flow rate along the dialyzer outlet line 193 within a target range. In some implementations, the dialysis pump 190 is controlled to maintain the flow rate along the dialyzer outlet line 193, as detected by the flow sensor 195, within a target range while simultaneously controlling the pressure along the dialyzer inlet line 1193, as detected by the pressure sensor 194, to remain below a threshold pressure.

The dialysis filtration system 109 also includes a resistance valve 196 positioned along the dialyzer outlet line 193 between the flow sensor 195 and the perfusate reservoir 104. The resistance valve 196 is a pinch valve that is controlled based on the pressure signals generated by the pressure sensor 194 along the dialyzer inlet line 192 and flow rate signals generated by the flow sensor 195 along the dialyzer outlet line 193. For example, the resistance valve 196 can be controlled to increase the pressure along the dialyzer outlet line 193 in response to determining, based on the pressure signal generated by pressure sensor 194 and the flow rate generated by the flow sensor 195, that the pressure within the dialyzer manifold 191 is below a threshold pressure. The dialyzer manifold 191 includes three dialyzers 1191, 1192, 1193 in order to provide redundancy in case one or more of the dialyzers 1191, 1192, 1193 becomes clogged or otherwise starts to function sub optimally during the perfusion process. For example, at the beginning of a perfusion process, the stopcocks 1196, 1198 upstream of a second dialyzer 1192 and a third dialyzer 1193, respectively, and the stopcocks 1197, 1199 downstream of the second dialyzer 1192 and the third dialyzer 1193, respectively, are closed. As a result, the perfusate fluid flowing along the dialyzer inlet line 192 is pumped through the first dialyzer 1191 without passing through the second dialyzer 1192 or third dialyzer 1193. Throughout perfusion process, signals generated by the pressure sensor 194 and flow sensor 195 are processed (e.g., by a machine learning module) to detect whether the one or more of the in-use dialyzers of the dialyzer manifold 191 that are becoming clogged or otherwise operating sub optimally. For example, at the beginning of perfusion, only the first dialyzer 1191 is in use and the first dialyzer 1191 can be determined to be operating sub optimally based on determining that (i) the pressure along the dialyzer inlet line 192, as determined based on signals generated by the pressure sensor 194, is above a threshold pressure and/or (ii) the flow rate of perfusate along the dialyzer outlet line 193, as determined based on signals generated by the flow sensor 195, is below a threshold flow rate. In response to determining that the first dialyzer 1191 is becoming clogged or otherwise operating sub optimally, the stopcocks 1196, 1197 upstream and downstream of the second dialyzer 1192 are opened to allow perfusate flowing along the dialyzer inlet line 192 to pass through the second dialyzer 1192. Further, the system can similarly detect, when the second dialyzer 1192 is in use, that the second dialyzer 1192 is becoming clogged or otherwise operating sub optimally based on signals generated by the pressure sensor 194 and/or the flow sensor 195. In response to detecting that both the first dialyzer 1191 and the second dialyzer 1192 becoming clogged or otherwise operating sub optimally, the stopcocks 1198, 1199 upstream and downstream of the third dialyzer 1193 are opened to allow perfusate flowing along the dialyzer inlet line 192 to pass through the third dialyzer 1193. The system 100 can similarly detect that the third dialyzer 1193 is becoming clogged or otherwise operating sub optimally based on signals generated by the pressure sensor 194 and/or the flow sensor 195. In response to determining that all three dialyzers 1191, 1192, 1193 of the dialyzer manifold 191 are clogged or otherwise operating sub optimally, the perfusion system 100 can generate an alarm and/or automatically shut down the dialysis filtration system 109 (e.g., by automatically stopping the pumping of the dialysis pump 190). By providing a dialyzer manifold 191 with three dialyzers 1191, 1192, 1193 that can be independently controlled, the dialysis filtration system 109 is capable of performing dialysis on perfusate flowing through the system 100 throughout extended periods of perfusion (e.g., 2 weeks or longer).

As filtered perfusate flows back to the perfusate reservoir 104 along the dialyzer outlet line 193, the waste filtered out of the perfusate flows along the waste line 197 to the in-line Raman spectroscopy system 107. Stopcocks 1188, 1189 are positioned along the waste line 197 and can be automatically controlled (e.g., using a servo-motor) to fluidly couple the dialyzers 1191, 1192, 1193 to the waste line 197. In addition, the stopcocks 1188, 1189 can be used to sample the waste filtered out of the perfusate by the dialyzer manifold 191. A resistance valve 1905 and a flow sensor 1907 are positioned along waste line 197 and is configured to control the rate of flow of waste between the dialyzer 191 and the in-line Raman spectroscopy system 107. For example, the resistance valve 1905 can be a pinch valve and, based on signals received from the flow sensor 1907, the resistance valve 1905 can be adjusted to increase or decrease the flow rate of waste fluid flowing into the in-line Raman spectroscopy system 107 in order to maintain a particular rate of fluid entering the in-line Raman spectroscopy system 107. In addition, the resistance valve 1905 can be controlled based on the pressure signals generated by pressure sensor 194 along the dialyzer inlet line 192 and the flow sensor 195 along the dialyzer outlet line 193. For example, the resistance valve 1905 can be controlled to increase the pressure along the waste line 197 in response to determining, based on the pressure signals generated by pressure sensor 194 and the flow rate signals generated by the flow sensor 195, that the pressure within the dialyzer 191 is below a threshold pressure.

As the brain is perfused with the perfusate, homeostasis and cellular activity of the brain 110 are maintained throughout the length of the perfusion process. In some implementations, the perfusion of the brain can be used to maintain homeostasis and cellular activity of the brain 24 hours or longer (e.g., 24-48 hours, 48-72 hours, 48-96 hours, 96 hours or longer, one week or longer, two weeks or longer, etc..) post-mortem. As a result of the restored metabolic and cellular activity provided by the perfusion of the brain, chemical exchanges and reactions occur within the brain 110 throughout the perfusion process, and these reactions can be monitored in real-time using the perfusion system 100. For example, as depicted in FIG. 1, the perfusion system 100 includes an in-line Raman spectroscopy system 107 (also referred to herein as “spectroscopy system 107”) that is configured to perform Raman spectroscopy on perfusate flowing through the system 100.

In some implementations, physiology and function of a brain subjected to extended perfusion (e.g., for 24 hours, 14 days, or longer) using the system 100 can be assessed based on several metrics. For example, physiology and functionality of the perfused brain can be assessed based on arteriovenous gradients demonstrating consistent consumption of glucose and oxygen with concurrent production of CO₂ and lactate and a physiological drop in pH within an expected range. In some implementations, physiology and functionality of the perfused brain can be determined based on detecting normalization of Na⁺ and K⁺ into physiological ranges. In some implementations, physiology and functionality of the perfused brain can be assessed based on normoxia quantified by comparison of brain measurements to known population variability in living human subjects, including normalization of hypoxia gene signatures and/or the hypoxia gene signatures not being upregulated. In some implementations, normal physiology and functionality of the perfused brain can be confirmed based on detecting pathway-stress Z-scores within ±1 standard deviation (SD) of baseline for most cell types. In some implementations, physiology and functionality of the perfused brain can be assessed based on measuring cellular viability. In some implementations, physiology and functionality of the perfused brain can be assessed based on conducting activity assays for neurons by measuring neuronal activity in post-perfusion brain slices measured using multielectrode arrays, detecting normal rates of anterograde and retrograde transport and/or measuring active cellular processes, including endocytosis and gene expression. In some implementations, physiology and functionality of the perfused brain can be assessed based on conducting activity assays for microglia responsiveness, for example, by performing immunogenic injection and measurement of inflammatory chemokines and cytokines. In some implementations, normal physiology and functionality of the perfused brain can be confirmed based determining that more than 80% of tricarboxylic acid (TCA) metabolites are within ±15% of a 0 hour reference measurement. In some implementations, normal physiology and functionality of the perfused brain can be confirmed based on determining that cerebral perfusion pressure remains within targeted bounds for more than 95% of the perfusion runtime. In some implementations, normal physiology and functionality of the perfused brain can be confirmed based on determining that cerebral metabolic rate of oxygen (CMRO₂) is maintained within ±10% of a 1 hour baseline measurement for more than 90% of the perfusion runtime. In some implementations, normal physiology and functionality of the perfused brain can be confirmed based on determining that there has been no reduction to the length of the corpus callosum (LCC) of the perfused brain or reduction to the anterior-posterior diameter (APD) of the perfused brain. In some implementations, normal physiology and functionality of the perfused brain can be confirmed based on performing histology to determine that normal mitochondrial morphology and preserved cytoarchitecture has been preserved in multiple brain areas. In some implementations, normal physiology and functionality of the perfused brain can be confirmed based on determining that there is no reduction in neocortical axon bundle density or change in axon orientation of the perfused brain. In some implementations, physiology and functionality of the perfused brain can be assessed based on determining that there is no global brain activity as measured by electrocorticography.

The in-line Raman spectroscopy system 107 includes a pump 111, a Raman sample line 123 fluidly coupling the pump 111 to a dialysis solution receptacle 117, and Raman spectrometer probe 119 configured to perform Raman spectroscopy on perfusate flowing through the Raman sample line 123. A stopcock 127 is positioned along the Raman sample line 123 upstream of the pump 111. The stopcock 127 can be used to sample the perfusate flowing from the dialysis filtration system 109 to the-line Raman spectroscopy system 107, for example, in order to conduct tests on the perfusate flowing to the-line Raman spectroscopy system 107 to calibrate the Raman spectrometer 119. In some implementations, the data generated by the Raman spectrometer 119 (e.g., predicted lactate or glucose concentrations in the perfusate generated by the Raman spectrometer 119) is compared to offline measurements (e.g., offline blood gas analysis of the perfusate to determine the concentration of lactate or glucose in the perfusate solution) in order to calibrate the Raman spectrometer 119. In some implementations, the root mean square error (RMSE) and R² values can be calculated for each analyte detected by the Raman spectrometer 119.

In order to accurately perform Raman spectroscopy, the perfusate in the Raman chamber is maintained within a predetermined temperature range that is suitable for Raman spectroscopy. As depicted in FIG. 1, the Raman spectroscopy system 107 includes a heat exchanger 115, a heat exchanger block 113, and a temperature sensor 121 positioned downstream of the heat exchanger block 113 and the heat exchanger 115. As perfusate flows along the Raman sample line 123, the temperature of the perfusate is measured by the temperature sensor 121, the perfusate is provided to the heat exchanger block 113, and the heat exchanger 115 is controlled based on the temperature signals generated by the temperature sensor 121 to adjust the temperature of the perfusate in the sample line 123 downstream of the heat exchanger block 113 to be within the predetermined range. For example, the heat exchanger 115 can be configured to continually adjust (e.g., raise or lower) the temperature of the perfusate flowing through the heat exchanger block 113 along the Raman sample line 123 until the temperature of the perfusate downstream of the heat exchanger block 113 measured by the temperature sensor 121 is within the predetermined temperature range. In some implementations, the heat exchanger 115 is configured to adjust the temperature of the downstream of the heat exchanger block 113 to be approximately 26 °C.

The Raman spectroscopy system 107 can be used to perform spectroscopy on the perfusate flowing out of the dialysis filtration system 109. For example, as depicted in FIG. 1, the waste line of the dialysis filtration system 109 that extends from the dialyzer manifold 191 is fluidly coupled to the Raman sample line 123 and, in order to perform Raman spectroscopy on the perfusate flowing from the dialyzer manifold the stopcock 127 is controlled (e.g., using a servo-motor) to fluidly couple the waste line 197 to the Raman sample line 123 and the pump 111 is operated to draw perfusate from the dialysis filtration system 109, along the waste line 197 and the Raman sample line 123, through the heat exchanger 115, and across the Raman spectrometer probe 119. The Raman spectrometer 119 is controlled to perform Raman spectroscopy analysis on the perfusate flowing through the Raman sample line 123 past the Raman spectrometer to analyze the chemical composition of the perfusate and identify one or more molecules present in the perfusate. In some implementations, the Raman spectrometer 119 is configured to capture high-resolution spectra at 1Hz. In some implementations, the Raman spectrometer 119 is configured to capture high-resolution spectra at 0.1Hz. Once the Raman spectrometer 119 has completed performing spectroscopy on the perfusate sample flowing along the sample line 123, the stopcock 127 is closed and the pump 111 is controlled to pump the perfusate along the Raman sample line 123 towards a dialysis solution receptacle 117.

As the perfusate flowing from the perfusate reservoir 104 is filtered by the dialysis filtration system 109, large molecules in the perfusate (e.g., oxygen carrier molecules, such as Lumbricus terrestris erythrocruorin (LtEc)) are directed back to the perfusate reservoir, and the filtered solution flowing along the waste line 197 contains small molecules that are contained within the perfusate, the presence of which can be detected using Raman spectroscopy. By performing Raman spectroscopy on the filtered perfusate solution flowing out of the dialysis filtration system 109, the chemical composition and proportions of components of the perfusate can be confirmed and adjusted, if needed.

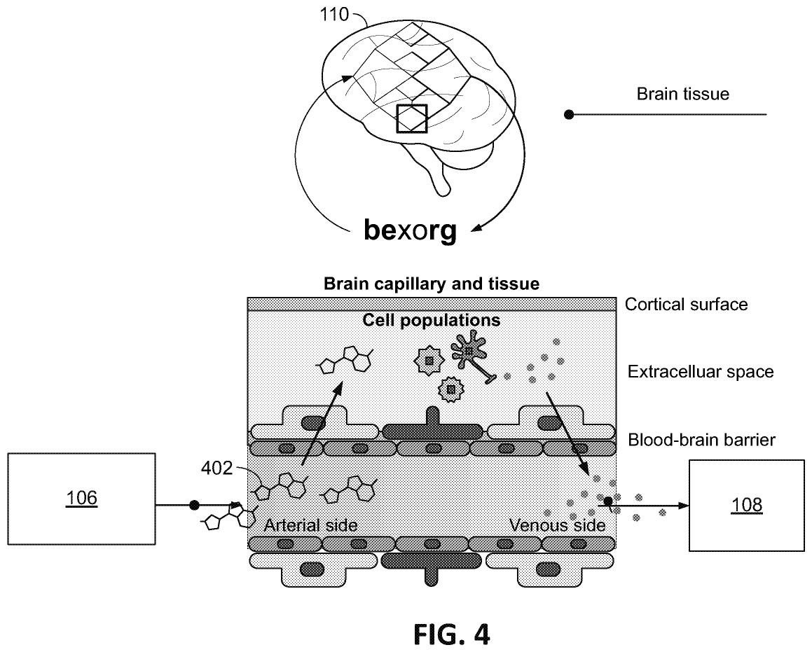

In addition, because the Raman spectroscopy system 107 is fluidly coupled to the perfusate reservoir 104 downstream of the brain 110, the uptake of chemical compounds by the brain 110 from the perfusate can be determined. For example, the chemical composition of the fresh perfusate provided to the brain 110 is predefined and can be confirmed by performing Raman spectroscopy on the fresh perfusate in the perfusate reservoir 104 using the spectroscopy system 107 prior to connecting the brain to the perfusion system 100. The spectroscopy system 107 can then be used to determine the chemical composition of the perfusate flowing throughout the perfusion system 100 (including flowing through the brain 110) in real-time. The known composition of the fresh perfusate provided to the brain 110 can be compared to the chemical composition of the perfusate flowing through the perfusion system in real-time, as determined using the spectroscopy system 107, in order to identify any differences in chemical composition in the fresh perfusate and used perfusate. The identified differences between the fresh perfusate and used perfusate can be used to determine various metabolic processes performed by the brain 110 during perfusion. As an example, the fresh perfusate provided by the brain 110 can include a particular concentration of a particular molecule, and the concentration of the same molecule in the perfusate flowing through the system during perfusion of the brain 110 can be determined using the Raman spectroscopy system 107. In response to determining that the concentration of the particular molecule is higher in the fresh perfusate than in the perfusate currently flowing through the perfusion system 100 and perfusing the brain 110, it can be determined that the particular molecule crossed the blood-brain barrier and that there has been uptake of the particular molecule by the brain 110. As depicted in FIG. 4, the blood-brain barrier selectively regulates passage of molecules 402 from the bloodstream (or perfusate) to the brain 110 through the use of specific proteins, such as transporters and tight junction proteins. In addition, the percentage of uptake of the particular molecule by the brain 110 can be determined by comparing the concentration of the particular molecule in the fresh perfusate and the concentration of the particular molecule in the used perfusate determined using the Raman spectroscopy.

Further, by comparing the chemical composition of the fresh perfusate provided to the brain 110 with the chemical composition of the used perfusate flowing through the perfusion system 100, as determined using the spectroscopy system 107, metabolic activity of the brain 110 and the health of the brain 110 can be determined in real-time during the perfusion process. For example, based on Raman spectroscopy on the used perfusate using the spectroscopy system 107, it can be determined that the used perfusate flowing out of the brain 110 and through the perfusion system 100 includes one or more chemical compounds that are not present in the fresh perfusate, which indicates that the brain 110 has reacted to one or more components of the perfusate. In addition, based on performing Raman spectroscopy on the used perfusate using the spectroscopy system 107, the response of the brain 110 to the perfusate can be determined, and other related side effects of the response of the brain 110 can be determined.

The perfusion system 100 also includes a sensor module 2107 that includes a colorimeter 1116, two photometers 1117, 1118, and a fluorimeter 1119. The sensor module 2107 is fluidly coupled to the in-line Raman spectroscopy system 107 and is configured to perform various tests on the filtered perfusate solution flowing out of the dialysis filtration system 109 and the in-line Raman spectroscopy system 107. As fluid flows along the sample line 123, the sensors 1116, 1117, 1118, 1119 of the sensor module 2107 each test one or more characteristics of the perfusate flowing through the sample line 123. For example, the colorimeter 1116 detects the colors of the perfusate flowing through the sample line 123. The photometers 1117, 1118 are configured to detect oximetry data related to the perfusate. For example, the photometers 1117, 1118 are each configured to detect a specific respective range of wavelengths and, based on comparing the data generated by each of the photometers 1117, 1118, the relative abundance of various chemical species within the perfusate can be determined. For example, based on comparing the output of each of the photometers 1117, 1118, the ratio of oxygen carrier protein (e.g., LtEc) oxidation can be determined.

The fluorimeter 1119 is configured to perform assays on the perfusate solution flowing along the sample line 123 in order to detect the presence and concentration of one or more molecules in the perfusate. In some implementations, a fluorescent dye and/or a fluorescently labeled species is included in the perfusate flowing to the brain 110 and the fluorimeter 1119 can be used to detect the presence of the fluorescent dye and/or fluorescently labeled species within the perfusate flowing out of the brain 110 and the data generated by the fluorimeter 1119 can be used to perform one or more biochemical assays on the perfusate.

Each of the sensors 1116, 1117, 1118, 1119 of the sensor module 2107 continuously operates throughout the perfusion process and provides sensor data characterizing the perfusate in real-time throughout perfusion. In addition, based on the data generated by the sensors 1116, 1117, 1118, 1119 of the sensor module 2107 during perfusion, quality control summaries showing signal stability, drift metrics, and sensitivity thresholds can be generated.

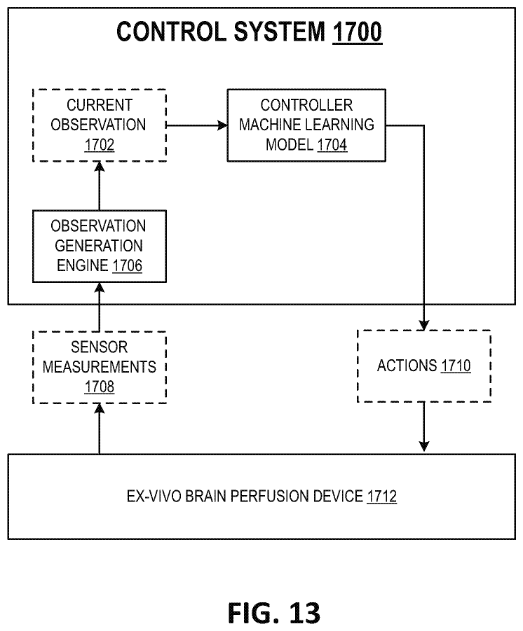

The data generated by the sensor module 2104 can be provided together with the data generated by one or more of the Raman spectroscopy system 107, the camera 1520, the environment sensor 1522, the sensor probes 128 along the sensor line 1124, the sensor block 124, the pressure sensor 141, and the flow sensor 142 to a trained machine learning model. As described in further detail in reference to FIGS. 13-15, based on the sensor data, the trained machine learning can determine the composition and rheological state of the perfusate and can infer hidden cerebrovascular states (e.g., dynamic resistance of the brain 110, vascular tone of the brain, etc.) that govern mass transfer and metabolic extraction, thereby transforming raw sensor data into actionable physiological metrics for closed-loop control to optimize function of the brain 110 during the perfusion process. Further, the machine learning model can link potential actuator moves (e.g., hardware setting control and adjustment of perfusate chemical composition) to predicted shifts in both mechanical outputs of the system 100 and metabolic outputs of the brain 110.

For example, based on the data generated by the Raman spectroscopy system 107 and the sensor module 2107, the machine learning model can determine the chemical composition of the perfusate flowing through the system 100 and can determine one or more adjustments to the chemical composition of the perfusate to improve viability of the brain 110 and extend the length of perfusion. For example, based on the data generated by the Raman spectroscopy system 107 and the sensor module 2107, the machine learning model can identify a concentration of a particular metabolite present in the perfusate below a particular threshold and, based on the metabolite concentration, can automatically open the stopcock 1149, open the solenoid valve 149, and control a syringe pump in the syringe pump manifolds 167, 1167 containing the corresponding metabolite to introduce the metabolite into the perfusate in circulation in the system to increase the concentration of the metabolite. For example, in response to determining, based on the data generated by the Raman spectroscopy system 107 and the sensor module 2107, that the level of glucose in the perfusate is below a threshold, the system can send command signals to open the stopcock 1149 and the solenoid valve 149 and can control the glucose syringe pump 1167 to add glucose to perfusate reservoir. In some implementations, the system 100 is configured to result in 10% or less of the glucose within the perfusate being depleted during a 24 hour continuous perfusion when the perfusion is performed at a physiological cerebral metabolic rate of oxygen (CMRO₂).

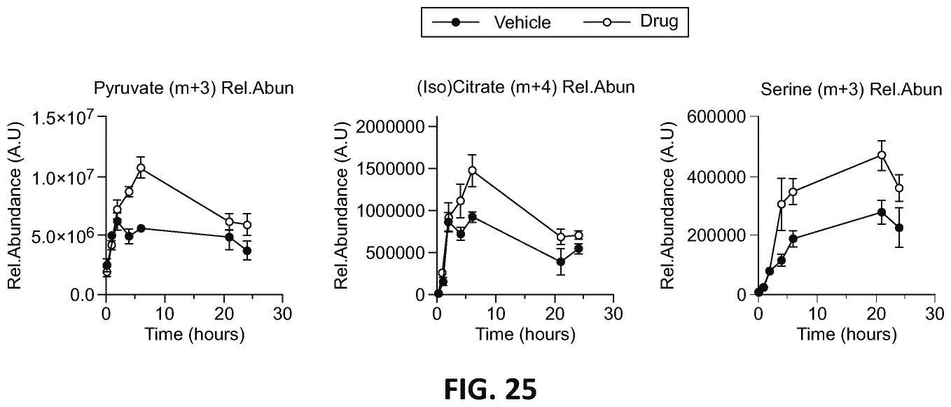

The glucose syringe pump manifold 1167 includes one or more syringe pumps configured to deliver glucose to the perfusate in the perfusate reservoir. The syringe pump manifold 167 includes one or more syringe pumps that are each configured to deliver a chemical component to the perfusate in the perfusate reservoir 104. For example, the syringe pump manifold 167 can be configured to add substances to the perfusate including, but not limited to, oxygen carrier molecules (e.g., LtEc or perfluorocarbon derivatives), a pH buffer (e.g., sodium bicarbonate), electrolytes, and hormones (e.g., insulin, calcitonin). For example, the syringe pump manifold 167 can be configured to add stable-isotope to the perfusate in the perfusate reservoir, such as, but not limited to, radiolabeled glucose. For example, referring to FIG. 25, in an example experiment, the syringe pump manifold 167 delivered ¹³C-glucose to the perfusate. Replacing glucose with ¹³C-glucose allowed for measurements of metabolites newly synthesized from glucose. Brains were treated systemically with vehicle and a drug that targeted metabolic processes. The drug significantly increased levels of pyruvate and citrate, particularly at early timepoints, indicating an increased capacity for energy production and providing a pharmacodynamic biomarker for target engagement, as depicted in FIG. 25. The drug also unexpectedly increased serine levels, providing novel insights to the mechanism of action of the therapeutic drug. Analysis of these metabolites as an endpoint, as would be the standard in in vivo models, rather than as a time course as provided in FIG. 25, would not have detected such significant effects.

Based on the. data generated by the Raman spectroscopy system 107 and the sensor module 2107, the machine learning model can determine that reducing the concentration of one or more metabolites could potentially improve viability of the brain tissue and extend the length of perfusion. In response to this determination, the system can automatically control the dialysis filtration system 109 to cause the dialysis pump 190 to pump perfusate from the perfusate reservoir 104 through the dialyzer manifold 191 and return the filtered perfusate to the perfusate reservoir 104.

Additionally, or alternatively, the fresh perfusate pump 1906 can be controlled to flow fresh (“base”) perfusate from the supplementary fresh perfusate reservoir 1903 into the perfusate reservoir while simultaneously flowing perfusate from the venous circuit 108 to the waste container 145 in order to increase or decrease the concentration of a particular metabolite in the perfusate (e.g., to adjust the pH or conductivity of the perfusate in the perfusate reservoir 104). For example, data generated by the Raman spectroscopy system 107 and/or the sensor module 2107 can be processed by a machine learning module to determine the chemical composition of the perfusate and can be combined with other data generated by sensors of the system (e.g., camera 1520) to determine a viability of the tissue of the brain 110. Based on this information, the machine learning module can determine one or more adjustments to be made to the chemical composition of the perfusate to improve brain tissue viability and extend the length of perfusion. In order to achieve the adjustment to the chemical composition of the perfusate, the machine learning module can generate command signals to control the fresh perfusate pump 1906 can be controlled to flow a particular amount fresh perfusate from the supplementary fresh perfusate reservoir 1903 into the perfusate reservoir 104 while simultaneously flowing perfusate from the venous circuit 108 to the waste container 145. By adding fresh perfusate to the reservoir 104 while simultaneously withdrawing used perfusate from circulation to the waste container 145, the concentration of the metabolites in the perfusate in circulation can be dynamically adjusted. The supplementary fresh perfusate reservoir 1903

The waste container 145 is positioned on a waste scale 157 configured to record the weight of perfusate contained in the waste container 145. Similarly, the perfusate reservoir 104 is positioned on a fresh perfusate scale 1910 to record the weight of perfusate contained in the perfusate reservoir 104. The fresh perfusate pump 1906 is controlled to pump fresh perfusate from the fresh perfusate reservoir 1903 into the perfusate reservoir 104 based on signals received from a flow sensor 199, signals received from the fresh perfusate scale 1910, and signals received from the waste scale 157. For example, based on the signals received from the waste scale 157 indicating an amount of fluid removed from the brain housing 102 into the waste container 145, the fresh perfusate pump 1906 can be controlled to provide a corresponding amount of fresh perfusate from the fresh perfusate reservoir 1903 into the perfusate reservoir 104, as determined based on the signals received from the flow sensor 199 and the fresh perfusate scale 1910. In some implementations, the fresh perfusate pump 1906 is controlled to pump fresh perfusate from the fresh perfusate reservoir 1903 to the perfusate reservoir 104 at the same rate that perfusate is drained to the waste container 145, as determined based on signals generated by the flow sensor 199 and the waste scale 157.

In some implementations, the fresh perfusate pump 1906 is controlled to pump fresh perfusate from the fresh perfusate reservoir 1903 to the perfusate reservoir 104 at predetermined times throughout perfusion and the solenoid valve 147 along the drain line 143 is simultaneously operated to allow an equal amount of perfusate to drain from the brain housing 102 to the waste container 145, as determined based on signals generated by the fresh perfusate scale 1910 and the waste scale 157. In some implementations, the fresh perfusate pump 1906 is controlled, based on signals received from the scale 1910 under the perfusate reservoir 104 and the scale 157 under the waste container 145, to continuously pump fresh perfusate from the fresh perfusate reservoir 1903 to the perfusate reservoir 104 while simultaneously draining an equal amount of perfusate from the venous circuit 108 into the waste container 145.

In some implementations, the functionality and effectiveness of controlled delivery of fresh perfusate from the supplementary fresh perfusate reservoir 1903 into the perfusate reservoir 104 at scheduled intervals or in response to sensor-defined chemical thresholds is validated based on time-series chemical sensor data generated by the system 100 (e.g., data relating to changes in (1) the concentration of bicarbonate detected in the perfusate, (2) the conductivity of the perfusate, (3) the concentration of lactate in the perfusate). For example, the time-series chemical sensor data generated by the system 100 can be used to confirm that controlled delivery of fresh perfusate from the supplementary fresh perfusate reservoir 1903 into the perfusate reservoir 104 maintains or restores the target chemical composition for the perfusate within 10% of the baseline perfusate values with a perfusion lasting 24 hours or longer.

As previously discussed, by passing the perfusate through the dialyzer manifold 191, small molecules within the perfusate (e.g., metabolites) are flowed to the dialysis solution receptacle 117, while large molecules in the perfusate (e.g., oxygen carrying molecules) are returned to the perfusate reservoir 104 along the dialyzer outlet line 193. Based on data received from the Raman spectroscopy system 107, the flow sensor 195 positioned downstream of the dialyzer 191, and a scale 2117 positioned under the dialysis solution receptacle 117, the machine learning model can determine the balance of small molecules vs. large molecules present in the perfusate in the perfusate reservoir 104. In addition, based on data received from the system sensors (e.g., sensor probes 128, sensor block 124, camera 1520, Raman probe 119, and/or sensor module 2107), the machine learning model can determine adjustments to the balance of small molecules vs. large molecules present in the perfusate to improve brain tissue viability and extend the length of perfusion.

For example, in response to the machine learning model determining, based on sensor data, that the concentration of small molecules within the perfusate should be increased, the machine learning model can generate command signals to operate a compensate pump 2108 to flow the filtered perfusate contained in the dialysis solution receptacle 117 to the perfusate reservoir 104 along a dialysis solution line 2109 fluidly coupling the dialysis solution receptacle 117 to the perfusate reservoir 104. The compensate pump 2108 can be operated to continue pumping perfusate from the dialysis solution receptacle 117 to the perfusate reservoir 104 until a particular amount of solution has been pumped out of dialysis solution receptacle 117, as determined based on signals generated by scale 2117 positioned under the dialysis solution receptacle 117.

Conversely, in response to the machine learning model determining, based on sensor data, that the concentration of small molecules within the perfusate should be decreased, the machine learning model can generate command signals to operate the dialysis pump 190 to draw perfusate solution from the perfusate reservoir 104 through the dialyzer manifold 191 to filter the perfusate and to direct small molecules filtered by the dialyzer manifold 191 to the dialysis solution receptacle 117. The dialysis pump 190 can be operated to continue pumping perfusate from the perfusate reservoir 104 to the dialysis solution receptacle 117 until a particular amount of solution has been added to the dialysis solution receptacle 117, as determined based on signals generated by the scale 2117 positioned under the dialysis solution receptacle 117.

As depicted in FIG. 1, the system 100 includes an impeller 198. The impeller 198 can be operated to mix the perfusate solution contained within the perfusate reservoir 104 to ensure that substances added to the perfusate reservoir (e.g., chemical components added via the syringe pump manifold(s) 167, 1167) are incorporated into the perfusate before flowing the perfusate from the perfusate reservoir 104 to the arterial circuit 106. In some implementations, the impeller 198 is operated continuously throughout the perfusion process. In some implementations, the impeller is controlled to operate for a predetermined amount of time after substances are added to the perfusate reservoir (e.g., from the syringe pump manifolds 167, 1167, from the supplementary base perfusate reservoir 1903, from the dialysis solution receptacle 117, etc.). By utilizing the impeller 198, the perfusate contained in the perfusate reservoir 104 can rapidly reach a homogenous state following the addition of one or more solutions into the perfusate reservoir 104. In some implementations, the efficacy of the impeller 198 to generate a homogenous solution can be tested (e.g., during calibration of the system 100) by analyzing data regarding the chemical composition of the perfusate generated by the sensor block 124, the sensor probes 128, the Raman spectroscopy system 109 and/or the sensor module 2107. For example, after adding one or more components to the perfusate reservoir 104 using the syringe module 1167, the data generated by the sensor block 124, the sensor probes 128, the Raman spectroscopy system 109 and/or the sensor module 2107 can be analyzed to determine the time required to reach stable concentrations of added chemical components in the perfusate downstream of the perfusate reservoir when using impeller mixing compared to when the impeller is not used to mix the perfusate in the perfusate reservoir 104.

In addition to determining the response of the brain 110 to the chemical composition of perfusate solution and the rheological properties of the perfusate solution in real-time during the perfusion process, the perfusion system 100 can also be used to test the pharmacodynamics of pharmaceutical compounds by the brain 110 in real-time, such as the rate of uptake of the pharmaceutical compounds by the brain and the chemical and metabolic reaction of the brain 110 to the pharmaceutical compounds. For example, as previously discussed, the perfusion system 100 includes a syringe pump manifold 167 that is configured to support one or more syringes and a stepper motor that is configured to control movement of a respective plunger of each of the syringes contained within the manifold 167. The syringe pump manifold 167 is fluidly coupled to the perfusate reservoir 104 along a drug input line 169. One or more of the syringes contained within the syringe pump manifold can be filled with a pharmaceutical compound, and the syringe pump manifold 167 is controlled throughout the perfusion process to add the pharmaceutical compound to the fresh perfusate in the perfusate reservoir 104 at a predetermined rate. For example, the syringe pump manifold 167 can be controlled to add a pharmaceutical compound into the perfusate at a rate that corresponds to the release profile for oral administration or intravenous administration of the pharmaceutical compound to a human.

The syringe pump manifold 167 is communicably coupled to the Raman spectrometer 119, and the rate of release of the pharmaceutical compound by the syringe pump manifold into the perfusate in the perfusate reservoir 104 can be controlled based on Raman spectroscopy analysis of the perfusate in the reservoir 104 by the spectroscopy system 107. As the syringe pump manifold 167 operates to deliver a pharmaceutical compound into the perfusate inside the perfusate reservoir 104, the concentration of the pharmaceutical compound in the fresh perfusate can be determined in real-time by performing Raman spectroscopy using the Raman spectroscopy system 107 on perfusate sampled from the reservoir 104. Based on the concentration of the pharmaceutical compound in the perfusate detected by the Raman spectroscopy system 107, the syringe pump manifold 167 can be controlled to increase, decrease, or maintain the rate the pharmaceutical compound is provided to the perfusate reservoir 104 in order to align the concentration of the pharmaceutical compound within the perfusate with the desired release profile in real-time.

In addition, the dialysis filtration system 109 can be used in conjunction with the Raman spectroscopy system 107 to provide a desired release profile of the pharmaceutical compound to the brain 110. As previously discussed, the perfusate system 100 is a closed-loop system. Therefore, any pharmaceutical compound added to the perfusate that is not metabolized by the brain 110 is recirculated within the perfusate. However, the release profile for many pharmaceutical compounds increases to a peak concentration, and then subsequently decreases. Therefore, in order to simulate certain release profiles, the concentration of the pharmaceutical compound within the perfusate needs to be reduced following a peak concentration. The dialysis filtration system 109 can be operated to remove the pharmaceutical compound from the perfusate at a controlled rate in order to cause the concentration of the pharmaceutical compound within the perfusate to decrease corresponding to the desired release profile. For example, as previously discussed, the concentration of the pharmaceutical compound in perfusate contained in the perfusate reservoir 104 can be determined in real-time by performing Raman spectroscopy using the Raman spectroscopy system 107 on perfusate sampled from the reservoir 104. Based on the concentration of the pharmaceutical compound in the fresh perfusate detected by the Raman spectroscopy system 107, the dialysis filtration system 109 can be controlled (e.g., based on the output of a machine learning model) to decrease the concentration of the pharmaceutical compound in the perfusate at a predetermined rate to align the concentration of the pharmaceutical compound with the desired release profile for the pharmaceutical compound. For example, based on the concentration of the pharmaceutical compound detected in the fresh perfusate by the Raman spectroscopy system 107, the dialysis pump 190 can be controlled to pump perfusate from the perfusate reservoir 104 along the dialyzer inlet line 192 to the dialyzer 191 and the dialyzer 191 removes at least a portion of the pharmaceutical compound from the perfusate. The perfusate with a reduced concentration of the pharmaceutical compound exits the dialyzer 191 and is pumped back to the perfusate reservoir 104 along the dialyzer outlet line 193. The dialysis pump 190 can be controlled to continue flowing perfusate through dialyzer 191 until the Raman spectroscopy system 107 detects that the concentration of the pharmaceutical compound in the perfusate within the perfusate reservoir 104 is below a concentration corresponding to the desired release profile. In some implementations, the flow rate of the dialysis pump 190 is controlled in real time based on the level of pharmaceutical compound within the perfusate detected by the Raman spectroscopy system 107. For example, the data generated by the Raman spectroscopy system 107 can be provided to a machine learning model and, based on the Raman spectroscopy data, the machine learning model can generate control commands to operate the dialysis pump 190. In some implementations, the dialysis pump 190 is controlled to flow perfusate from the perfusate reservoir 104 at a predetermined rate and for a predetermined duration in order to remove the pharmaceutical compound at a rate corresponding to the desired release profile.

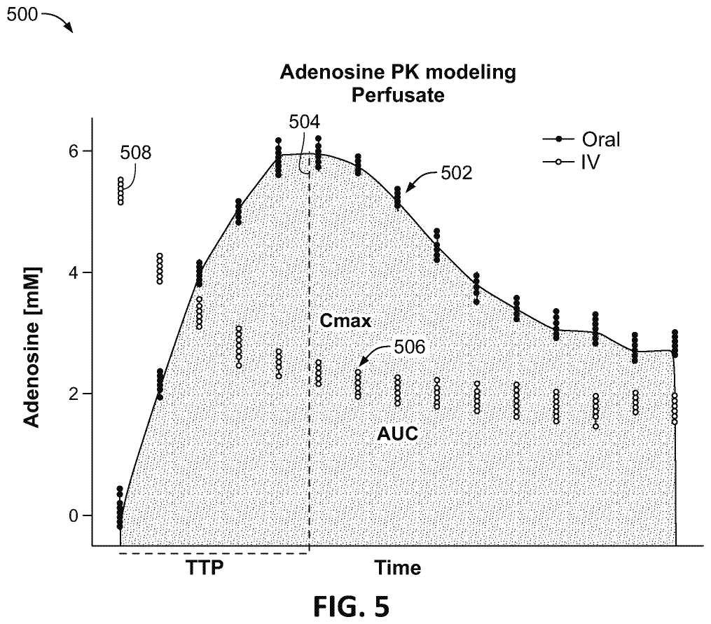

FIG. 5 depicts Raman spectroscopy data 500 generated during example perfusion procedures in which adenosine was added to the perfusate during the perfusion procedure. During a first perfusion procedure, the syringe pump manifold 167 and dialysis filtration system 109 were controlled to adjust the concentration of adenosine within the perfusate in the perfusate reservoir 104 to correspond to a typical release profile 502 for oral administration of adenosine. As can be seen in FIG. 5, the oral administration release profile 502 defines an initial increase in concentration of adenosine until the concentration of adenosine reaches a peak concentration 504, followed by a decrease in concentration of adenosine. In order to administer adenosine to a brain being perfused by the system 100 according to the oral release profile 502, the syringe pump manifold 167 was initially controlled to provide adenosine to the perfusate in the perfusate reservoir 104. As the syringe pump manifold 167 operated to add adenosine to the perfusate in the perfusate reservoir 104, samples of the perfusate from the perfusate reservoir 104 were periodically tested using the Raman spectroscopy system 107, as described above. Based on the Raman spectroscopy data 500, the concentration 502 of adenosine within the fresh perfusate in the perfusate reservoir 104 was tracked in real-time and the rate of the syringe pump manifold 167 was periodically adjusted (e.g., using control commands generated by a machine learning model) to reduce the rate at which the concentration of adenosine increased, in accordance with the portion of the release profile 502 before the peak concentration 504.

Once the concentration of adenosine in the perfusate in the perfusate reservoir 104 was identified as being equal to the peak concentration 504, as determined based on the Raman spectroscopy data 500 generated by performing Raman spectroscopy system 107, the dialysis filtration system 109 was operated (e.g., using control commands generated by a machine learning model) to remove adenosine from the perfusate at a controlled rate to gradually decrease the concentration of adenosine in the perfusate, as depicted in the portion of the release profile 502 after the peak concentration 504. As the dialysis filtration system 109 was operated to remove adenosine from the perfusate, samples of the perfusate from the perfusate reservoir 104 were periodically tested using the Raman spectroscopy system 107, as described above. Based on the Raman spectroscopy data 500, the concentration 502 of adenosine within the fresh perfusate in the perfusate reservoir 104 was tracked in real-time and the dialysis filtration system 109 was controlled (e.g., using control commands generated by a machine learning model) to progressively reduce the rate at which the concentration of adenosine within the perfusate decreased, as depicted in the portion of the release profile 502 after the peak concentration 504.

During a second perfusion procedure on a second brain, the syringe pump manifold 167 and dialysis filtration system 109 were controlled to cause the concentration of adenosine within the perfusate in the perfusate reservoir 104 to correspond to a typical release profile 506 for intravenous (IV) administration of adenosine. As can be seen in FIG. 5, the IV administration release profile 506 defines a peak initial increase concentration 508 of adenosine that decrease exponentially. In order to administer adenosine according to the IV release profile 506 to a brain being perfused by the system 100, the syringe pump manifold 167 was controlled to provide an initial bolus of adenosine into the perfusate in the perfusate reservoir 104 to achieve the initial peak concentration 508 of adenosine in the perfusate. The initial peak concentration 508 of adenosine in the perfusate was confirmed by performing Raman spectroscopy on a sample of perfusate from the perfusate reservoir 104, as described above. Once the initial peak concentration 508 was provided to the perfusate by the syringe pump manifold 167, as confirmed based on the Raman spectroscopy data 500 generated by the Raman spectroscopy system 107, the dialysis filtration system 109 was operated (e.g., using control commands generated by a machine learning model) to remove adenosine from the perfusate at a controlled rate to exponentially decrease the concentration of adenosine in the perfusate in accordance with the IV release profile 506. As the dialysis filtration system 109 was operated to remove adenosine from the perfusate, samples of the perfusate from the perfusate reservoir 104 were periodically tested using the Raman spectroscopy system 107, as described above. Based on the Raman spectroscopy data 500, the concentration 506 of adenosine within the fresh perfusate in the perfusate reservoir 104 was tracked in real-time and the dialysis filtration system 109 was dynamically controlled (e.g., using control commands generated by a machine learning model) to exponentially reduce the rate at which the concentration of adenosine within the perfusate in accordance with the IV release profile 506.

Referring back to FIG. 1, as the pharmaceutical compound flows from the one or more syringes in the syringe pump manifold 167 into the perfusate in the perfusate reservoir 104, the perfusate with the pharmaceutical compound is simultaneously provided to the brain 110 from the perfusate reservoir 104 along the organ line 144 and the used perfusate exits the brain 110 into the basin 1022 of the brain housing 102, as previously described. The Raman spectroscopy system 107 can be controlled to perform Raman spectroscopy on the used perfusate flowing out of the brain housing 102 and into the perfusate reservoir 104 in order to determine the effects of the pharmaceutical compound on the brain 110. For example, at predetermined intervals throughout the perfusion process the dialysis pump 111 is operated to draw used perfusate from perfusate reservoir 104 along the Raman sample line 123, through the heat exchanger block 113, and past the Raman spectrometer 119. As used perfusate flows through the Raman sample line 123, the Raman spectrometer 119 is controlled to perform Raman spectroscopy analysis on the used perfusate to analyze the chemical composition of the used perfusate. After passing past the Raman spectrometer 119, the used perfusate flows along the sample line 123 through the sensor module 2107 and the sensors 1116 1117, 1118, 1119 perform assays to on the used perfusate to analyze the chemical composition of the used perfusate. The effect of the pharmaceutical compound on the brain 110 can be determined based on the chemical composition of the used perfusate determined by the Raman spectroscopy system 107 and the sensor module 2107. For example, the spectroscopy and chemical analysis of the used perfusate can be used to determine the concentration of the pharmaceutical compound present in the used perfusate. In response to detecting that the concentration of pharmaceutical compound in the used perfusate flowing out of the brain 110 is lower than the concentration of the pharmaceutical compound in the fresh perfusate in the perfusate reservoir 104 at a corresponding time, it can be determined that the brain 110 has metabolized (e.g., “taken up”) at least a portion of the pharmaceutical compound. As a result, the concentration of the pharmaceutical compound in the used perfusate detected by analyzing the data generated by Raman spectroscopy system 107 and sensor module 2107 (e.g., using a machine learning model) can be used to determine whether the pharmaceutical compound is capable of crossing the blood-brain barrier and the rate at which the brain 110 has metabolized the pharmaceutical compound.

The used perfusate flowing out of the brain 110 can also be analyzed by the Raman spectroscopy system 107 and sensor module 2107 (e.g., using a machine learning model) to determine one or more molecules generated by the brain 110 in response to the to the pharmaceutical compound. For example, prior to adding a pharmaceutical compound to the perfusate, the chemical composition of the used perfusate flowing out of the brain 110 can be analyzed by the Raman spectroscopy system 107 and sensor module 2107 (e.g., using a machine learning model) in order to form a baseline composition. After the pharmaceutical compound has been introduced to the perfusate and flowed to the brain 110, the chemical composition of the used perfusate flowing out of the brain 110 can be analyzed by the Raman spectroscopy system 107 and sensor module 2107 (e.g., using a machine learning model) and compared to the baseline chemical composition for the used perfusate to determine one or more molecules that are present in the used perfusate flowing out of the brain 110 following the introduction of the pharmaceutical compound that were not present prior to the introduction of the pharmaceutical compound. The molecules produced by the brain 110 in response to the introduction of the pharmaceutical compound and detected in the used perfusate based on the data generated by the spectroscopy system 107 and sensor module 2107 can be used to determine the reaction of the brain 110 to the pharmaceutical compound, the likely efficacy of the pharmaceutical compound on treating the targeted disease, and potential side effects that may result from the pharmaceutical compound. As a result, potential new drugs can be tested using the perfusate system 100 to accurately determine their likely effectiveness in treating the target disease in humans.

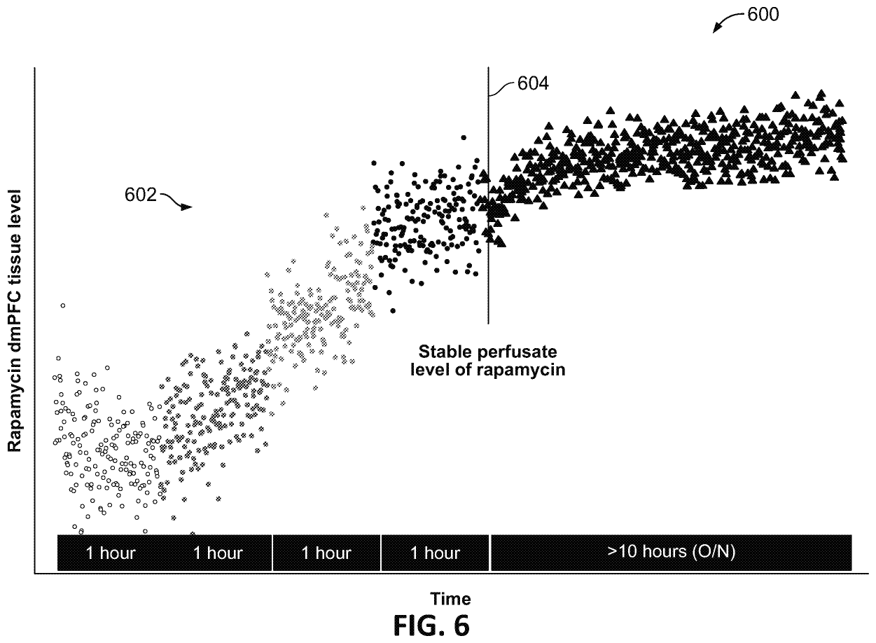

FIG. 6 depicts Raman spectroscopy data 500 generated during an example perfusion procedure in which the drug rapamycin was provided to the brain 110. During perfusion of the brain 110 using the perfusion system 100, the syringe pump manifold 167 was controlled (e.g., using command signals generated by a machine learning model) to provide rapamycin to the perfusate in the perfusate reservoir 104 at a particular rate, which was then provided to the brain 110 along the organ line 144. As the perfusate containing the rapamycin was provided to the brain 110, Raman spectroscopy was performed on the tissue of the dorsomedial prefrontal cortex (dmPFC) of the brain 110. As can be seen from the Raman spectroscopy data 600 in FIG. 6, the concentration 602 of rapamycin present in the brain 110 continually increased until a stable concentration 604 was reached. Based on the Raman spectroscopy data 600 indicating the presence of rapamycin in the tissue of the dmPFC of the brain 110, it was determined that the rapamycin in the perfusate was able to cross the blood-brain barrier of the brain 110. In addition, based on the Raman spectroscopy data 600, the rate of uptake and metabolism of rapamycin by the brain 110 for the particular release profile of rapamycin provided by the perfusate system 100 was determined.

Referring to FIG. 1, the perfusate system includes a computer system 188 that is communicably coupled to the electrical components of the perfusate system 100 including, but not limited to the pumps 111, 114, 184, 190, 1002, 1122, 1906, 2108, the heat exchangers 115, 118, the oxygenator 116, the gas mixer 120, the electronic pressure regulator 166, the sensor block 124, the sensor probes 128, the level sensors 125, 180, the pressure sensors 141, 164, 168, 194, 1008, 1168 the flow sensors 130, 142, 195, 199, 1107, 1907 the temperature sensor 121, the environmental sensor 1522, the camera 1520, the resistance valves 152, 196, 1905, the syringe pump manifolds 167, 1167, the scales 157, 174, 1910, 2117 the solenoid valves 133, 147, 149, 151, 158, 171, 173, 178, 1120, 1121, the stopcocks 127, 132, 135, 155, 159, 181, 182, 1109, 1111, 1113, 1115, 1169, 1171, 1175, 1181, 1183, 1188, 1189, 1194, 1195, 1196, 1197, 1198, 1199, the Raman spectrometer 119, and the Raman spectroscopy probe 172. In some implementations, the computer system 188 can be in wireless or wired communication with the electronic components of the perfusate system 100. In some implementations, a display 189 of the computing device 188 is configured to display data generated by the perfusate system 100. For example, sensor data generated by the sensor probes 128 can be transmitted to the computer system 188 in real-time, and the display 189 can be configured to display sensor data received from the sensor probes 128. In some implementations, the data generated by the perfusate system 100 (e.g., the data sensor data generated by the sensor block 124, the sensor probes 128, the Raman spectroscopy system 107, the sensor module 2107, the flow sensors 130, 142, 195, 199, 1107, 1907, the pressure sensors 141, 164, 168, 194, 1008, 1168, the environment sensor 1522, and/or the camera 1520) is transmitted in real-time to the computer system 188, and the computer system 188 is configured to transmit the data generated by the perfusate system 100 to a remote database for storage. The computer system 188 is also configured to communicably couple and controls the electronic components of the perfusate system 100 to perform the perfusion process as described herein. For example, the computer system 188 is configured to receive data from the pressure sensor 141 and the flow sensor 142, and based on the sensor data, control the second pressure regulator 168 to adjust the pulsatile flow provided by the pulse generator 136 along the organ line 144.

In some implementations, the perfusion system 100 is configured to be provided with fully modular hardware architecture in which one or more of the sub-systems of the perfusion system 100 (e.g., the in-line mechanical filtration system 1001, the dialysis filtration system 109, the Raman spectroscopy system 107, the syringe pump manifolds 167, 1167, etc.) can be integrated as discrete, plug-and-play components during platform manufacturing and assembly. For example, in some implementations, greater than 90% of the mechanical components of the perfusion system 100 can be interchanged with a replacement part removed from another unit (i.e., another perfusion system 100). In some implementations, standardized electrical, fluidic, and software interface protocols are used across the various sub-systems of the perfusion system 100.

In addition, the perfusion system 100 can be provided together with comprehensive digital schematics and software toolkits that support the scalable manufacturing, modular assembly, and consistent operation of the perfusion system 100. For example, an operator of the perfusion system 100 can be provided with documented support for rapid reconfiguration and troubleshooting of the perfusion system 100 at the sub-system level, including comprehensive, step-by-step documentation (i.e., instructions) for assembling the perfusion system platform 100, ensuring reproducibility across all operators of the system. The assembly instructions provided with the perfusion system 100 can include (1) a complete bill of materials (BOM) with part numbers, suppliers, and specs, (2) detailed Computer-Aided Design (CAD) schematics and wiring diagrams, and (3) sequential assembly and testing procedures with visual aids. In some implementations, manufacturers of the perfusion system 100 are provided with step-by-step build documentation for manufacturing the perfusion system 100,, ensuring reproducibility across engineering and manufacturing teams.

Further, as previously discussed, the perfusion system 100 is configured to be calibrated and tested using a standardized quality control (QC) protocols for assessing operational effectiveness and deviations from target parameters for the perfusion system 100. For example, the perfusion system 100 is configured to perform in-line quality control assessments for flow, pressure, temperature, pH, and signal responsiveness based on predefined quality control benchmarks. In some implementations, the quality control assessments for the perfusion system 100 define acceptable threshold deviations for each core metric of the perfusion system 100. For example, in some implementations, the quality control benchmarks for the perfusion system 100 require that the flow rate measured by the flow sensors of the system 100 deviate 5% or less from a reference flow rate measurement. In some implementations, the quality control benchmarks for the perfusion system 100 require that the temperature measured by the temperature sensors of the deviate 0.5 °C or less from a reference temperature measurement. In some implementations, the perfusion system 100 includes a startup validation suite of software that walks an operator of the system 100 through quality control checking and calibration, and the startup validation suite of software stores the pass/fail criteria for each of the quality checks to be performed on the perfusion system 100 and includes mechanisms for digitally logging and storing the results of the quality checks performed on the system 100.

An example process of calibrating the perfusion system 100 and performing perfusion of a brain 110 using the system 100 will now be described with reference to FIGS. 1-2D and 4.

Prior to performing a perfusion procedure, reusable components of the perfusion system 100 that come into contact with the perfusate during a perfusion process are sterilized (e.g., using autoclave) including, but not limited to, the pulse generator 136, the perfusate reservoir 104, the sensor block 124 and sensor probes 128, the brain housing 102, the Raman heat exchanger block 113, the oxygenator 116, the pressure sensors 141, 164, 168, 194, 1008, 1168, and the flow sensors 130, 142, 195, 199, 1107, 1907. After sterilization of the relevant system components is complete, each of the components of the system 100 is positioned inside a clean room that houses the perfusion system 100 throughout the perfusion process. In some implementations, the clean room that houses the perfusion system 100 is provided with a positive pressure system that forces positive pressure into the clean room to prevent contamination of the perfusion system. Once the reusable components of the perfusion system 100 have been sterilized and positioned in the clean room, the various fluid lines are attached to the system 100 as depicted in FIG. 1 to form the arterial circuit 106, the venous circuit 108, the in-line Raman spectroscopy system 107, and the dialysis filtration system 109. In some implementations, the fluid lines of the arterial circuit 106, the venous circuit 108, the in-line Raman spectroscopy system 107, and the filtration system 109 are configured to withstand 24 hours or longer of continuous pumping with a flow rate deviation of 5% or less, with preserved wall integrity (e.g., no micro-cracks, bulging, or loss of elasticity), and with particle counts in the fluid inside the fluid line being below contamination thresholds, indicating minimal shedding.

Once components of the perfusion system 100 are positioned in the clean room and fluidly coupled as depicted in FIG. 1 and prior to adding perfusate to the system, the air supply system 138 and the pressure sensor 141 along the organ line 144 are calibrated. For example, the resistance valve 152 along the organ line 144 and the solenoid valve 158 along the brain housing shunt line 156 and stopcock 159 along the organ line 144 are closed, and a flow of pressurized air is provided from the air source 160 to the first pressure regulator 162. The first pressure regulator 162 regulates the air flow to a pressurized air stream having a pressure of about 5 psi. The pressurized air stream is then provided from the electronic pressure regulator 162 to the second pressure regulator 166. As previously discussed, the second pressure regulator 166 has tighter tolerances compared to the first electronic pressure regulator 162 and is configured to provide a pressurized air stream to the air inlet 230 of the pulse generator 136 at a particular pressure (e.g., 5 psi). The smoothed, pressurized air stream flows from the second pressure regulator 166, through the pulse generator 136, along the organ line 144, and to the brain housing 102. As the pressurized air stream flows from the second pressure regulator 166 along the air supply line 161 and the organ line 144, the pressure of the air stream is measured by the second pressure sensor 168 along the air supply line 161 and the pressure sensor 141 along the organ line 144. The pressure measured by the second pressure sensor 168 along the air supply line 161 is compared to the pressure measured by the pressure sensor 141 along the organ line 144, and the pressure sensor 141 along the organ line 144 is calibrated based on any differences between the pressure measured by the second pressure sensor 168 along the air supply line 161 and the pressure measured by the respective pressure sensor 141 along the organ line 144.

In some implementations an NIST certified manometer 1068 can be fluidly coupled to the air calibration line 163 and is used to calibrate the pressure sensor 141 along the organ line 144. For example, the organ line 144 is pressurized with air provided along the air calibration line 163 and the stopcock 155 upstream of the pressure sensor 141 and the stopcock 159 downstream of the pressure sensor 141 are both closed to generate a stable pressure between the stopcocks 155, 159 and along the air calibration line 163. With the stopcocks 155, 159 close, the pressure along the organ line 144 is measured by the pressure sensor 141 and the pressure along the air calibration line 163 is measured by the manometer 1068. The pressure detected by the manometer 1068 is compared to the pressure detected by the pressure sensor 141 and is used to calibrate the pressure sensor 141. In some implementations, the pressure sensor 141 is calibrated using the manometer 1068 to yield 2% or less deviation from the NIST certified manometer 1068 reading across the operational pressure range for the organ line 144. In addition, the pressure sensor 141 can be calibrated to ensure that drift of the pressure sensor 141 output remains below a predetermined threshold over the course of a particular operational period (e.g., drift of the pressure sensor 141 output can be configured to be less than 0.5% over a 30 minute period). The other pressure sensors 164, 168, 194, 1008, 1168 of the perfusion system 100 can be similarly tested by comparing the output of the respective pressure sensors 164, 168, 194, 1008, 1168 by comparing the output of the respective sensors 164, 168, 194, 1008, 1168 with the output of an NIST certified manometer fluidly coupled to the respective fluid line across the operational pressure range of the perfusion system 100.

In some implementations, the pressure sensor 141 downstream of the resistance valve 152 is further calibrated using air provided from the second pressure regulator 166 along the air calibration line 163. For example, pressurized air is provided from the second pressure regulator 166 to the organ line 144 along the air calibration line 163 through stopcock 155. The stopcock 155 upstream of the pressure sensor 141 and the stopcock 159 downstream of the pressure sensor 141 are each closed to generate a stable air pressure along the organ line 144 between the stopcocks 155, 159 corresponding to the air pressure provided by the second pressure regulator 166. Once the stopcocks 154, 155 are closed, the pressure sensor 141 can be calibrated based on any difference in the pressure measured by the pressure sensor 141 can be compared to the pressure provided by the second pressure regulator 166.

Once the air supply system 138 and the pressure sensor 141 along the organ line 144 are calibrated, fresh perfusate is added to the perfusate reservoir 104. In some implementations, the perfusate is sterilized using ultraviolet irradiation prior to being provided to the perfusate reservoir 104. Once the perfusate reservoir 104 is filled with fresh perfusate, the system 100 is controlled to flood the fluid lines of the arterial circuit 106, the venous circuit 108, the Raman spectroscopy system 107, and the dialysis filtration system 109 with fresh perfusate in order to remove any air contained within the fluid lines. In order to “flood” the system 100 with perfusate and purge air from the fluid lines of the system 100, the solenoid valves and stopcocks of the arterial circuit 106, the venous circuit 108, the Raman spectroscopy system 107 are opened, and the flow pump 114, the dialysis pump 190, the filter pump 1002, the sensor line pump 1122, the sample pump 184, the compensate pump 2108, the supplement pump 1906, and the pump 111 of the Raman spectroscopy system 107 and the pulse generator 136 are each controlled to flow perfusate from the perfusate reservoir 104 through fluid lines of the arterial circuit 106, the venous circuit 108, the Raman spectroscopy system 107, the filtration system 1114, the dialysis filtration system 109, and the sensor module 2107, as described in detail above.

The perfusate flows through the fluid lines to the sensor block 124 and the brain housing 102. Once the level sensor 180 in the brain housing 102 detects that the brain housing 102 is filled to a desired level with perfusate, the solenoid valve 178 between the brain housing 102 and the perfusate reservoir 104 and the solenoid valve 1121 along the venous sensor line 1129 are opened and the sensor pump 1122 is operated to pump perfusate from the brain housing 102 along the housing outlet line 176 and the sensor line 1124 back to the perfusate reservoir 104. Similarly, once the level sensor 125 in the sensor block 124 detects that the sensor block 124 is filled to a desired level with perfusate, the solenoid valves 149, 151 between the sensor block 124 and the perfusate reservoir 104 open to allow perfusate to flow from the sensor block 124 to the perfusate reservoir 104.

As the perfusate flows through the system 100, the air contained within the fluid lines is vented out of the fluid lines into a head space at the top of the perfusate reservoir 104, a head space at the top of the sensor block 124, and a head space at the top of the brain housing 102. The pumps 111, 114, 184, 190, 1002, 1122, 1906, 2108 and pulse generator 136 continue to operate to pump perfusate throughout the system 100 until all of the air is vented out of the fluid lines.

In addition, the air supply system 138 is configured to provide a pulsatile stream of pressurized air to the pulse generator 136 sufficient to cause the pulse generator 136 to force any air contained in the central housing 202 of the pulse generator 136 downstream to the sensor block 124 or the brain housing 102. The stopcock 159 along the organ line 144 is opened and fluidly couples the pulse generator 136 to the perfusate reservoir 104 along a debubble line 1136 during the flooding of the fluid lines. As the pulse generator 136 operates to flow fluid along the organ line 144, air contained in the pulse generator 136 and the organ line 144 is pushed through the organ line 144, along the debubble line 1136, and into the head space at the top of the sensor block 124.

Once the arterial circuit 106, the venous circuit 108, the Raman spectroscopy system 107, and the dialysis filtration system 109 have been flood with perfusate, the solenoid valves 133, 158 along the oxygenator shunt line 131 and the brain housing shunt line 156 are closed, and an initial sample of the perfusate is collected using one of the and/or the automatic sampler system 105. The initial (“zero hour”) sample of the perfusate can be tested using various techniques (e.g., spectroscopy, gene sequencing, cytokine analysis, anti-body analysis, genomic analysis, transcriptomic analysis, proteomic analysis, metabolomic analysis) in order to determine baseline measurements for the perfusate, including the chemical composition of the perfusate. For example, a user can use other laboratory equipment to test the initial perfusate sample collected from the system 100, for example, to perform gene sequencing, cytokine analysis, anti-body analysis, genomic analysis, transcriptomic analysis, proteomic analysis, metabolomic analysis. In addition, an initial sample of the perfusate can be tested using the Raman spectroscopy system 107, the sensor probes 128, the sensor block 124, and/or the sensor module 2107, as described herein, and the data generated by the sensors of the system 100 can be processed (e.g., by a machine learning model) to determine one or more characteristics about the initial (“base) perfusate, including the chemical composition of the perfusate.

Once the system 100 is flooded with perfusate, the sensor probes 128, the sensor block 124, Raman spectrometer, and the sensor 1116, 1117, 1118, 1119 of the sensor module 21107 are controlled to take initial sensor readings, which are used to calibrate the sensors 124, 128, 1119, 1116, 1117, 1118, 1119. In some implementations, the sensor probes 128 are calibrated using electronic calibration devices, such as the i-STAT analyzer device manufactured by Abbott.

Once the system 100 is flooded with perfusate and the sensors 124, 128, 1119, 1116, 1117, 1118, 1119 have been calibrated, the initial conditions for the gas mixer 120 and heat exchanger 118 are set, and the oxygenator 116, heat exchanger 118, and gas mixer 120 are operated to optimize the temperature and gas composition of the perfusate for the initial stage of perfusion of a brain. As previously discussed, the heat exchanger 118 and gas mixer 120 are controlled based on signals generated by one or more sensor probes 128 and the sensor block 124. For example, the heat exchanger 118 is controlled to adjust the temperature of the perfusate to be within a predetermined range of temperature based on the temperature signals received from the sensor probes 128. In addition, the gas mixer 120 is controlled to adjust the amount of oxygen, nitrogen, and/or carbon dioxide in the perfusate within the oxygenator 116 based on one or more signals generated by the sensor probes 128 indicating the dissolved oxygen concentration of the perfusate, the dissolved nitrogen concentration of the perfusate, and the dissolved carbon dioxide concentration of the perfusate. In some implementations, the gas mixer 120 is configured to adjust the amount of oxygen, nitrogen, and/or carbon dioxide in the perfusate to be within a predetermined range, as detected by one or more sensor probes 128.

In some implementations, the readings from the sensor probes 128 are analyzed to determine that key physiological and environmental control subsystems—including temperature regulation, oxygenation, and dialysis exchange—can reach and maintain target parameters prior to attachment of the brain 110 to the system. For example, data generated by the sensor probes 128 can be analyzed to determined that the final-state parameters for temperature, blood oxygen concentration, conductivity, and pH of the perfusate fall within acceptable physiological ranges (e.g., 36-37°C, >90% oxygen saturation, pH of 7.35-7.45). In some implementations, the system is configured to maintain oxygen saturation of the perfusate within 10% of a target oxygen saturation. In some implementations, the readings from the sensor probes 128 are analyzed to determine that the target parameters for temperature, dissolved oxygen concentration, conductivity, and pH of the perfusate can be maintained by the system for a predefined stabilization period (e.g., 5 minutes or longer).

Once the system 100 is flooded with perfusate and the temperature and gas composition of the perfusate have been adjusted to the correct initial settings, the system 100 enters a “zero flow stage” in which the pumps 111, 114, 184, 190, 1002, 1122, 1906, 2108 are controlled to stop pumping the perfusate system 100 and the resistance valve 152 along the organ line 144 is closed for a predetermined amount of time. In some implementations, the system is controlled to be in the zero-flow stage for approximately 5 minutes. While the system is in the zero-flow stage, there is no flow through the flow sensors 130, 142, 195, 199, 1107, 1907 and the flow sensors 130, 142, 195, 199, 1107, 1907 are calibrated during the no flow state. In some implementations, one or more of the flow sensors 130, 142, 195, 199, 1107, 1907 is calibrated by comparing the flow values measured by each of the flow sensors 130, 142, 195, 199, 1107, 1907 against a standardized scale-based reference (e.g., gravimetric measurement using calibrated lab scale). In some implementations, one or more of the flow sensors 130, 142, 195, 199, 1107, 1907 are configured to generate flow error rates of 5% or less compared to a standardized flux reference. In some implementations, one or more of the flow sensors 130, 142, 195, 199, 1107, 1907 is tested by the respective flow sensor 130, 142, 195, 199, 1107, 1907 taking multiple measurements across a range of flow rates to confirm linearity and repeatability of the sensor response.

Once the flow sensors 130, 142 along the arterial circuit 106 have been calibrated, the pumps 114, 190, 111 are controlled to resume pumping perfusate through the system 100 and the pulse generation system 1302 is controlled to achieve a target pressure and flow rate along the organ line 144, as described in detail above.

While the pumps 114, 190, 111 and the pulse generator 136 are operated and before the brain 110 is fluidly connected to the organ line 144, a quality control evaluation of the system 100 can be performed. For example, while operating the pumps 114, 190, 111 and the pulse generator 136 and before the brain 110 is fluidly connected to the organ line 144, the stopcock 159 positioned between the organ line 144 and the brain housing 102 is closed and the solenoid valve 158 along the brain shunt line 156 is opened to direct fluid flowing along the organ line 144 through the brain shunt line 156 and into the brain housing 102. The brain shunt line 156 has a standardized length and diameter, which results in a known, standardized pressure and flow rate through the brain shunt line 156. As such, the flow rate measured by the flow rate sensor 142 and the pressure measured by the pressure sensor 141 can be compared to the expected, standardized pressure and flow rate through the brain shunt line 156 to determine whether one or more components of the system 100 are not functioning properly. For example, if the flow rate measured by the flow sensor 142 while pumping perfusate through the brain shunt line 156 deviates from the standard flow rate for the brain shunt line, the flow sensor 142 may be recalibrated to eliminate the deviation between expected flow rate and measured flow rate (e.g., either by an operator of the system 100 or automatically). Similarly, if the pressure measured by the pressure sensor 141 while pumping perfusate through the brain shunt line 156 deviates from the standard pressure for the brain shunt line, the pressure sensor 142 may be recalibrated to eliminate the deviation between expected pressure and measured pressure (e.g., either by an operator of the system 100 or automatically).

In addition, while the pumps 114, 190, 111 and the pulse generator 136 are operated, initial measurements of one or more characteristics of perfusate can be performed before attaching the brain 110 to the system 100 in order to determine one or more baseline characteristics of the perfusate. These initial readings can include Raman spectroscopy analysis data generated by performing Raman spectroscopy on the perfusate using the Raman spectroscopy system 107, data generated by the sensor block 124, data generated by the sensor probes 128, and/or data generated by the sensor module 2107, as described herein. In some implementations, samples of the perfusate 402, 404 are collected and the samples of the perfusate 402, 404 can be tested manually to determine one or more characteristics of the perfusate. In some implementations, the stopcocks are manually operated by a user of the system 100 to collect perfusate samples 402, 404. In some implementations, the stopcocks are automatically controlled using servo-motors operated to collect perfusate samples 402, 404. In some implementations, the samples of the perfusate are automatically collected by the automatic sampler system 105. The initial readings of the perfusate can also include measurements performed by the sensor probes 128, the sensor block 124, the Raman spectroscopy system 109, and/or the sensor module 2107.

Once the initial measurements of the perfusate characteristics have been performed, the system 100 is controlled to drain the perfusate from the brain housing 102. In order to drain the brain housing 102, the solenoid valve 147 along the drain line 143 is opened to direct perfusate inside the brain housing 102 along the housing outlet line 176 and the drain line 143 into the waste container 145. The waste scale 157 measures the weight of perfusate inside the waste container 145 in real-time as the brain housing 102 is drained. In response to the waste scale 157 detecting that a predetermined amount of perfusate has been provided to the waste container 145 from the brain housing 102 corresponding to the volume of the brain housing 102, the solenoid valve 147 along the drain line 143 is closed. Once the brain housing 102 is drained of perfusate, the brain housing scale 174 is zeroed out to calibrate the brain housing scale 174. Once the brain housing 102 is drained and the brain housing scale 174 is calibrated, the flow pump 114 and pulse generator 136 are controlled to pulse perfusate along the organ line 144 to provide a small amount of perfusate to the brain housing 102 prior to connecting the brain 110 to the system.

The brain 110 is then positioned in the brain housing 102 and fluidly coupled to the system 100 using the organ line 144. Prior to placing the brain 110 in the brain housing 102, samples of pig vasculature are sutured to the carotid arteries and basilar artery of the human brain 110 at a first end and are fluidically connected to the organ line 144 (e.g., through suturing) at a second end.

Once the brain 110 is positioned in the brain housing 102 and fluidly coupled to the arterial circuit 106, the flow pump 114 is controlled to flow perfusate through the oxygenator 116 to the pulse generator 136, and the air supply system 138 and the pulse generator 136 are controlled to pump perfusate to the sensor block 124 and to provide pulsatile flow of perfusate to the brain 110 along the organ line 144.

Perfusate flows out of the oxygenator 116 along the arterial fluid line 112 to the pulse generator 136. As previously described, due to the relative positioning of the pulse generator 136, the brain housing 102, and the sensor block 124, and the structure of the pulse generator 136, all of the perfusate provided to the pulse generator 136 from the arterial fluid line 112 passively flows through the sensor block inlet line 146 when the diaphragm 214 of the pulse generator 136 is in an unflexed (“neutral”) position. As perfusate passes through the sensor block 124, the sensor block 124 measures one or more characteristic of the fresh perfusate inside the housing 126, including, but not limited to, viscosity. At the same time, the solenoid valve 1120 along an arterial sensor line 1128 is open to fluidly couple the sensor line 1124 to the sensor block 124 and a peristaltic sensor pump 1122 is operated to draw perfusate from the sensor block 124 along the sensor line 1124 and through the sensor probes 128. The sensor probes measure one or more characteristic of the fresh perfusate inside the housing 126, including, but not limited to, pH, dissolved oxygen concentration, dissolved nitrogen concentration, dissolved carbon dioxide concentration, viscosity, and temperature. Based on the characteristics measured by the sensor block 124 and the sensor probes 128, the oxygenator 116, heat exchanger 118, and gas mixer 120 are controlled in order to optimize the temperature, O₂ level, and gas composition of the perfusate. In addition, based on the viscosity of the perfusate measured by the sensor block 124, the supplement pump 1906 can be controlled to flow fresh perfusate to from the supplementary fresh perfusate reservoir 1903 to the perfusate reservoir 104 and the solenoid valve 147 can be opened to drain perfusate to the waste container 145 in order to adjust the viscosity of the perfusate.

As previously discussed, the heat exchanger 118 is configured to adjust the temperature of the perfusate flowing through the heat exchanger 118 based on the temperature of the perfusate detected by the sensor probes 128. At the beginning of a perfusion procedure, the temperature of the perfusate is gradually increased by the heat exchanger 118 until a target temperature for the perfusate is reached, as detected based on the data generated by sensor probes 128. In some implementations, the temperature of the perfusate is increased at a rate of 0.1 °C/minute until a target temperature of the perfusate is reached, as detected by the sensor probes 128. In some implementations, the target temperature is 36 °C, and the heat exchanger 118 is controlled to increase the temperature of the perfusate from is 20 °C to is 36 °C over the course of a three hour period.

In addition, at the beginning of a perfusion procedure, the O₂ concentration of the perfusate is gradually increased by the gas mixer 120 until a target partial pressure of O₂ in the perfusate (pO₂) is reached, as detected based on the sensor probes 128. In some implementations, the target partial pressure of O₂ in the perfusate is 450 mmHg. For example, at the beginning of the perfusion process, the partial pressure of O₂ in the perfusate is 200 mmHg and, based on data generated by the sensor probes 128, the gas mixer 120 and oxygenator 116 are controlled to gradually increase the concentration of the O₂ in the perfusate until the partial pressure of O₂ in the perfusate is 450 mmHg. In some implementations, the O₂ concentration for the perfusate is increased at a rate of 1% increase in pO₂ every thirty minutes for the first 12 hours of perfusions and increases to a rate of 2% increase in pO₂ every thirty minutes until the end of the perfusion process. By gradually increasing the temperature and the O₂ concentration of the perfusate provided to the brain 110 at the beginning of the perfusion procedure, the internal resistance of the brain 110 is decreased, which results in improved perfusion of the brain 110 with reduced risk of injury to the brain 110.

While the temperature and O₂ concentration of the perfusate are being ramped up by the heat exchanger 118 and the gas mixer 120, the pulse generation system 1302 is simultaneously controlled to progressively increase the pressure of the perfusate provided to the brain 110 along the organ line 144 until a target flow rate of perfusate along the organ line 144 is reached. For example, as previously discussed, due to the relative positioning of the pulse generator 136, the brain housing 102, and the sensor block 124, and the structure of the pulse generator 136, none of the perfusate provided to the pulse generator 136 passively flows through the organ line 144 to the brain 110 when the diaphragm 214 of the pulse generator 136 is in an unflexed (“neutral”) position. Therefore, in order to provide pulsatile flow of perfusate to the brain 110 along the organ line 144, the air supply system 138 is controlled to provide pulsatile, pressurized air streams to the pulse generator 136 in order to cause the diaphragm 214 of the pulse generator 136 to flex downwards towards the fluid chamber 236, which causes the diaphragm 214 to cover the openings 210 in the fluid chamber 236 of the pulse generator 136 and increase the pressure within the interior 212 of the pulse generator 136. As result of the increased pressure caused by the flexing of the diaphragm 214, at least a portion of the fresh perfusate contained within the interior 212 of the pulse generator is forced through the organ line outlet 208 of the pulse generator 136 and along the organ line 144 to the brain 110.

At the beginning of the perfusion procedure, the pulse generation system 1302 is controlled to provide pulsatile flow of perfusate to the brain 110 along the organ line 144 at an initial pressure, as measured by the pressure sensor 141 along the organ line 144. For example, as previously discussed, the second pressure regulator 166 is controlled based on pressure signals generated by pressure sensor 141 along the organ line to adjust the frequency and duration which the second pressure regulator 166 provides pressurized air to the pulse generator 136 in order to adjust amplitude and/or base of the pulsatile flow of perfusate to the brain 110 until the initial pressure threshold along the organ line 144 is reached, as detected by the pressure sensor 141. In some implementation, the initial pressure threshold along the organ line is 16 mmHg.

Once the initial pressure threshold along the organ line 144 has been maintained for a predetermined period, as determined based on the signals generated by the pressure sensor 141 along the organ line 144, the second pressure regulator 166 is controlled to cause the pulse generator 136 to progressively increase the pulsatile flow along the organ line 144 until a particular target flow rate along the organ line 144 is detected by the flow sensor 142 along the organ line 144. In some implementations, the pulse generation system 1302 is controlled based on signals received from the pressure sensor 141 to increase the pressure along the organ line 144 at a rate of 0.0333 mmHg/minute. In some implementations, the pulse generation system 1302 is configured to increase the pressure in the organ line 144 from 16 mmHg to 20 mmHg, increasing the pressure along the organ line at a rate of 1 mmHg every 30 minutes. In some implementations, the pulse generation system 1302 in controlled to maintain a constant amplitude of pressure along the organ line 144. In some implementations, the pulse generation system 1302 in controlled to maintain a constant amplitude of 10 mmHg along the organ line 144.

Once the target flow rate is detected by the flow sensor 142, the pulse generation system 1302 is controlled based on signals generated by the flow sensor 142 in order to provide pulsatile flow along the organ line 144 sufficient to maintain the predetermined flow rate along the organ line 144 as detected by the flow sensor 142, while maintaining the pressure along the organ line 144 (as detected by pressure sensor 141) within a predetermined range of pressure. In some implementations, the target flow rate is 10 mL/minute. By gradually increasing the pressure and the flow rate of the perfusate provided to the brain 110, the internal resistance of the brain 110 is correspondingly gradually reduced, which enables the vasculature of the brain 110 to more effectively receive the perfusate while minimizing injury or other damage to the brain 110.

Perfusate flowing along the organ line 144 is provided to the brain 110 and perfuses the entire vasculature of the brain 110, including the penetrating arterioles, pre-capillary arterioles, and capillaries of the brain 110. After flowing through the vasculature of the brain 110, the used perfusate is collected in the basin 1022 of the brain housing 102 and exits the brain housing 102 along the housing outlet line 176.

As previously discussed, at the beginning of the perfusion of the brain 110, an initial “flush” of the brain 110 is performed in order to remove toxins and other unwanted chemical compounds that have built up in the brain 110 during the post-mortem period prior to perfusion During the initial flush of the brain 110, the solenoid valve 147 along the drain line 143 is open to direct used perfusate flowing out of the brain 110 to the waste container 145. Once the waste scale 157 detects that a predetermined amount of used perfusate has been provided to the waste container 145 during the initial flush, the solenoid valve 147 along the drain line 143 is controlled to close, which directs used perfusate flowing along the housing outlet line 176 to the perfusate reservoir 104. In some implementations, the brain 110 is initially flushed with 500 mL of perfusate.

In some implementations, after completing flush procedures, the perfusate is tested using the Raman spectroscopy system 107 and the sensor module 2107 to determine the chemical composition of the perfusate exiting the brain 110 following the initial flush of the brain. The post-flush measurements generated by the Raman spectroscopy system 107 and the sensor module 2107 can be used to confirm clearance of endogenous brain waste products, such as native hemoglobin, as a result of flush protocols, ensuring chemically clean startup conditions. For example, post-flush measurements generated by the Raman spectroscopy system 107 and the sensor module 2107 can be used confirm waste product concentrations within the perfusate flowing out of the brain 110 are below defined physiological or experimental thresholds. If the post-flush measurements indicate that waste product concentrations within the perfusate flowing out of the brain 110 are above predefined thresholds, the system 100 can be automatically controlled to perform additional flushing of the brain 110 until the sensor data generated by the Raman spectroscopy system 107 and the sensor module 2107 indicates that the waste product concentrations within the perfusate flowing out of the brain 110 have fallen below the threshold concentrations.

Once the initial flush of the brain 110 is complete and the solenoid valve 147 along the drain line 143 is closed, the perfusion system 100 is operated as a closed-loop system and the brain 110 is perfused with perfusate from the perfusate reservoir 104 throughout the duration of the perfusion procedure. As the brain 110 is being perfused, the dialysis filtration system 109 can be operated to remove metabolic waste products or other unwanted substances from the perfusate, as previously described herein. In some implementations, the brain 110 is perfused using the perfusion system 100 for 24 hours or longer (e.g., 24-48, 48-72 hours, 48-96 hours, 96 hours or longer, 1 week or longer, 2 weeks or longer).

In some implementations, during perfusion, the pulse generator 136 is controlled during the perfusion process based on iterative physiological-rhythmicity protocols that mimic blood circulation flow rates and heart rates. For example, at various points throughout the perfusion process, the pulse generator 136 can be operated to provide a pulsatile flow along the organ line 144 that mimic blood flow through the arterial system during specific activities including, but not limited to, sleeping (e.g., circadian cycles) and exercise.

During the perfusion process, the automatic sampler system 105 is controlled to collect samples of the fresh perfusate 302 in the arterial circuit 106 and the used perfusate in the venous circuit 108 at predetermined intervals. For example, the automatic sampler system 105 can be controlled to collect samples of fresh perfusate 302 from the perfusate reservoir 104 and used perfusate from the brain housing 102 at the start of a perfusion process (“0 hour”), 6 hours after the start of a perfusion process, 12 hours after the start of a perfusion process, and every 24 hours after the start of a perfusion process for the length of the perfusion process (e.g., for two weeks or longer). In order to collect a sample of fresh perfusate from the arterial circuit 106, the solenoid valve 151 along the arterial sample line 150 between the sensor block 124 and the perfusate reservoir 104 is closed, the solenoid valve 171 along the arterial sample line 150 between the sensor block 124 and the automatic sampler system 105 is opened, and the pump 184 of the automatic sampler system 105 is operated to draw perfusate out of the sensor block 124 along the arterial sample line 150, and the solenoid manifold 185 is controlled to direct the perfusate through the sample collection line 186 and into a sample collection vessel. In order to collect samples of the used perfusate 304 from the venous circuit 108,, the solenoid valve 178 along the housing outlet line 176 between the brain housing 102 and the perfusate reservoir 104 is closed, the solenoid valve 173 along the venous sample line 183 is opened, and the pump 184 of the automatic sampler system 105 is operated to draw perfusate out of the brain housing 102 along the venous sample line 183, and the solenoid manifold 185 is controlled to direct the perfusate through the sample collection line 186 and into a sample collection vessel.

During the perfusion process, the syringe pump manifolds 167, 1167 are controlled to introduce one or more chemical compounds into the perfusate reservoir 104, which are then provided to the brain 110 through the perfusate. As previously discussed, the syringe manifolds 167, 1167 can include syringes containing one or more chemical components of the perfusate and the syringe manifolds 167, 1167 can be automatically controlled to adjust the chemical composition of the perfusate in real-time throughout perfusion to improve brain tissue viability and extend the length of perfusion. For example, a machine learning model can receive data from various sensors of the perfusion system 100 (e.g., sensor probes 128, sensor block 124, camera 1520, Raman probe 119, and/or sensor module 2107) and can analyze the sensor data to determine the chemical composition of the perfusate and the health of the brain 110 being perfused by the system. Based on the determined chemical composition of the perfusate and the health of the brain 110, the machine learning module can generate command signals to automatically control one or more of the syringe manifolds 167, 1167 to introduce one or more chemical components into the perfusate reservoir 104 to adjust the chemical composition of the perfusate in order to improve brain tissue viability and extend perfusion length. Similarly, based on chemical composition of the perfusate and the health of the brain 110 determined by the machine learning model, the machine learning model can generate command signals to automatically control the dialysis pump 190 to pump perfusate from the perfusate reservoir 104 through the dialyzer manifold 191 to adjust the chemical composition of the perfusate (e.g., by filtering out one or more compounds from the perfusate) in order to improve brain tissue viability and extend perfusion length.

In some implementations, the syringe pump manifold 167 is operated in conjunction with the dialysis filtration system 109 to introduce a pharmaceutical compound to the brain 110 in accordance with a particular predetermined release profile. For example, as previously described, the Raman spectroscopy system 107 can be used to determine the concentration of the pharmaceutical compound in the perfusate contained in the perfusate reservoir 104 in real-time throughout the perfusion procedure. Based on the concentration of the pharmaceutical compound in the perfusate detected by the Raman spectroscopy system 107, the syringe pump manifold 167 can be controlled to increase, decrease, or maintain the rate the pharmaceutical compound is provided to the perfusate reservoir 104 in order to align the concentration of the pharmaceutical compound with the desired release profile. In addition, based on the concentration of the pharmaceutical compound detected in the perfusate by the Raman spectroscopy system 107, the dialysis pump 190 can be controlled to pump perfusate from the perfusate reservoir 104 along the dialyzer inlet line 192 into the dialyzer manifold 191, which removes at least a portion of the pharmaceutical compound from the perfusate. The perfusate with a reduced concentration of the pharmaceutical compound exits the dialyzer manifold 191 and is pumped back to the perfusate reservoir 104 along the dialyzer outlet line 193.

In addition, the Raman spectroscopy system 107 is controlled to perform Raman spectroscopy on the perfusate in the perfusate reservoir 104 at predetermined intervals throughout the perfusion process in order to determine one or more characteristics of the perfusate in real-time throughout the perfusion process. As discussed above, the data generated by the Raman spectroscopy system 107 can be used to control the syringe pump manifolds 167, 1167 and the dialysis filtration system 109 to produce adjust the chemical composition of the perfusate and a particular release profile of a pharmaceutical compound. In addition, the data generated by the Raman spectroscopy system 107 can be used to determine metabolic activity of the brain 110 and the health of the brain 110 in real-time during the perfusion process. For example, as previously discussed, the chemical composition of the used perfusate flowing out of the brain 110 can each be determined using the spectroscopy system 107, and various metabolic processes performed by the brain 110 during perfusion can be determined based on any differences in chemical composition in the fresh perfusate and used perfusate. In addition, while a pharmaceutical compound is being provided to the brain 110 through the perfusate, the Raman spectroscopy system 107 is controlled to perform Raman spectroscopy on samples of the used perfusate 404 flowing out of the brain 110 in order to determine whether the pharmaceutical compound has crossed the blood-brain barrier of the brain 110, the reaction of the brain 110 to the pharmaceutical compound, the likely efficacy of the pharmaceutical compound on treating the targeted disease, and potential side effects that may result from the pharmaceutical compound.

The Raman spectroscopy data generated by the Raman spectroscopy system 107 is stored in a database. In some implementations, the Raman spectroscopy data generated by the Raman spectroscopy system 107 is provided to the trained machine learning model to predict, e.g., a metabolic pathway of the pharmaceutical compound in the brain, an activation or deactivation of certain gene pathways in the brain, physiological changes to the brain (e.g., swelling, tissue damage, infection, etc.), relating to the administration of the pharmaceutical compound, and reaction of the brain 110 to changes in the chemical composition of the perfusate.

Throughout the perfusion process, the perfusion system 100 is capable of automatically detecting system faults and initiating safe shutdown procedures to isolate any malfunctioning sub-systems of the perfusion system 100 (e.g., the in-line mechanical filtration system 1001, the dialysis filtration system 109, the Raman spectroscopy system 107, the syringe pump manifolds 167, 1167, etc.) while continuing to maintain critical process parameters within operational limits in order to protect brain viability and maintain overall process stability. For example, throughout the perfusion process, the system 100 can analyze sensor data generated by the system 100 (e.g., using a machine learning module) to detect deviation from standard boundary values for parameters such as pressure, flow, temperature, and pH and identify the sub-system causing the detected deviations. The malfunctioning sub-system can be flagged (e.g., by generating an alarm or transmitting an alert to an operator of the system 100) and a record of the malfunction can be stored in a log (e.g., in a database) without affecting perform of the other sub-systems. In addition, the system 100 can be controlled to automatically and proactively shut down individual sub-systems that exceed safety thresholds to prevent cascading failures and ensure brain protection. During logging the malfunction event and shutting down the malfunctioning sub-system, the system 100 continues to operate to maintain core perfusion metrics (e.g., pressure along the organ line 144, perfusate temperature, and oxygen concentration in the perfusate) within respective target ranges during the malfunction event.

The perfusion system 100 is also configured to automatically restore failed sub-systems to a safe and stable operating state after faults have been resolved without the loss of critical data or uncontrolled fluctuations in the perfusion parameters while rebooting the previously malfunctioning sub-system. In some implementations, the perfusion system 100 is configured to restore malfunctioning sub-systems to validated baseline parameters once the respective failed sub-system is restored. In some implementations, the success restoration of the previously malfunctioning system can be confirmed based on analyzing sensor data to confirm that the perfusion operations have resumed within operational boundaries.

In some implementations, the ability of the perfusion system 100 to identify system malfunctions and proactively shut down individual sub-systems that exceed safety thresholds can be validated. For example, during or prior to calibration of the perfusion system, the system 100 can be subjected to simulated fault conditions and the ability of the system 100 to automatically shut down of the module that is being simulated as being compromised without impacting downstream sub-systems during or after the shutdown can be verified. In addition, subjecting the system 100 to a simulated fault condition can also be used to verify that they system 100 is capable of maintaining stable brain viability (e.g., based on sensor data indicating brain swelling and brain pressure) following intervention by the system 100 in response to the simulated fault condition.

Once the perfusion process is complete, for example, because the predetermined length of perfusion has elapsed, the pumps 111, 114, 184, 190, 1002, 1122, 1906, 2108 are each controlled to stop pumping the perfusate through the system 100, the brain 110 is removed from the brain housing 102, and the perfusate contained within the perfusate reservoir 104 and the brain housing 102 is disposed of in a waste container or directly to a drain. The fluid lines of the arterial circuit 106, the venous circuit 108, the in-line Raman spectroscopy system 107, and the dialysis filtration system 109 are disconnected from respective components of the system 100 and are replaced with new fluid lines prior to the start of another perfusion procedure.

The process of calibrating the perfusion system 100 and performing perfusion of a brain 110 using the system 100, including calibration, priming, de-bubbling, quality control routines, ramp-up, and waste ejection, is configured to be performed autonomously by the system 100. For example, the process to calibrate and to prime the system for connection to the brain can be fully automated and can include validating the system 100 for readiness using standardized metrics and thresholds. As a result, the system 100 can ensure that all subsystems meet operational benchmarks before brain connection and transition the system 100 into a perfusion-ready state with minimal manual intervention by an operator. In addition, the flow sensor and pressure sensor calibration can be automatically validated (“quality checked”) by the perfusion system 100 running a standardized quality control procedure that simulates a complete startup environment. The pass/fail criteria for the quality checks of the flow sensor and pressure sensor are clearly defined and logged in system records for each perfusion process. Further, the Raman spectrometer and the sensors 1116, 1117, 1118, 1119 of the sensor module 2107 can be automatically validated by the perfusion system 100 and validation reports for metabolic and chemical markers can be automatically generated across various perfusion timepoints.

In addition, based on analyzing data generated by the various sensors of the system in real-time a throughout perfusion, command signals can be generated (e.g., by a machine learning model) to control and adjust the mechanical components of the system to perform perfusion of a brain 110 using the system 100 with minimal manual intervention by an operator.

The machine learning model used to analyze sensor data and detect one or more responses of the brain 110 to the perfusate and the perfusion process can be implemented as any appropriate type of machine learning model, e.g., including one or more of: a neural network, or a random forest, or a support vector machine, or a linear regression model. Further, the machine learning model can have any appropriate architecture that enables the machine learning model to perform its described functions, e.g., processing a model input that is based on Raman spectroscopy of used perfusate that has passed through brain tissue to generate a prediction characterizing the brain. For example, for a machine learning model implemented as a neural network model, the architecture of the neural network can include any appropriate types of neural network layers (e.g., convolutional layers, attention layers, fully connected layers, recurrent layers, message passing layers, pooling layers, transformer layers, and so forth) in any appropriate number (e.g., 3 layers, or 10 layers, or 25 layers) and connected in any appropriate configuration (e.g., as a directed graph of layers).

The model input provided to the machine learning model can represent the Raman spectroscopy data as an ordered collection of numerical values, e.g., a vector, matrix, or other tensor of numerical values. For instance, the Raman spectroscopy data can include a wavelength axis, representing the energy shift from the laser light and an intensity axis representing the amount of scattered light at each energy level. The model input can include Raman spectroscopy data captured at one time point, or at a sequence of multiple time points, e.g., that are spaced at regular intervals in a time window. The model output generated by the machine learning model can be, e.g., a classification output (e.g., that classifies the model input into a category from a predefined set of categories), or a regression output (e.g., that predicts a continuous value based on the model input), or any other appropriate type of model output.

The machine learning model can be trained on a set of training examples by a machine learning training technique. Each training example can include: (i) a training input that includes Raman spectroscopy data generated by performing Raman spectroscopy on used perfusate that has passed through the brain tissue of the brain, and (ii) a target output defining a model output that should be generated by the machine learning model by processing the training input. The target output can characterize one or more characteristics of the brain, e.g., a metabolic pathway of a pharmaceutical compound that has been provided to the brain (e.g., in perfusate passed through the brain), an activation or deactivation of certain gene pathways in the brain, or physiological changes to the brain (e.g., swelling, tissue damage, infection, etc.). The target outputs included in the training examples may be generated based on experimental measurements or observations of brains during previous perfusion experiments.

In some implementations, the training examples include labelled training data generated during 10 or more independent perfusion processes performed in a vascular model (“vascular phantom”), and each perfusion process contains 600 or more cardiac cycles with complete pressure and flow traces. In some implementations, the training examples include sensor data and/or omics data obtained during perfusion of 50 or more brains (e.g., 100 brains, 500 brains, etc.) in order to account for variability in cerebrovascular hemodynamics across different, individual brains. In some implementations, the training examples include mechanical, hemodynamic, metabolic, Raman, and device log data generated by the system over the course of 100 or more hours of perfusion.

In some implementations, the machine learning model is trained to maintain a perfusion pressure within 10% bounds for 95% or more of a four hour perfusion (e.g., performed on a vascular phantom). In some implementations, the machine learning model is trained to reduce tone variance (□²) by 25% or more compared to the tone variance provided by a baseline proportional-integral-derivative controller. In some implementations, the system is configured to achieve 30% or greater reduction in vascular tone variance compared to historical, open-loop perfusion processes.

Training the machine learning model on the set of training examples can include, for each training example, training the machine learning model to reduce a discrepancy between: (i) a predicted output generated by processing the training input of the training example using the machine learning model, and (ii) the target output specified by the training example. More specifically, the machine learning model can be trained to optimize an objective function that, for each training example, measures an error between: (i) a predicted output generated by processing the training input of the training example using the machine learning model, and (ii) the target output specified by the training example. The objective function can measure the error, e.g., as a cross-entropy error, or as a squared error, or in any other appropriate way.

FIG. 13 shows an example control system 1700. The control system 1700 is an example of a system implemented as computer programs on one or more computers in one or more locations in which the systems, components, and techniques described below are implemented.

The control system 1700 is configured to automatically control aspects of the operation of the ex-vivo brain perfusion device 1712, e.g., to enable the ex-vivo brain perfusion device to perfuse a brain over an extended period of time while maintaining a level of homeostasis in the brain.

In particular, at each of multiple time steps, the control system 1700 can receive a set of sensor measurements that are generated by one or more sensors of the ex-vivo brain perfusion device 1712 and that characterize a current state of the brain being perfused by the ex-vivo perfusion device. The control system 1700 can process the sensor measurements 1708 to select one or more actions 1710 to be performed by the ex-vivo brain perfusion device 1712. The actions 1710 can include, e.g., actions to modify one or more chemical parameters of the perfusate being used by the ex-vivo brain perfusion device 1712 to perfuse the brain, or actions to modify one or more operational parameters of the ex-vivo brain perfusion device, as will be described in more detail below. The control system 1700 can transmit instructions to perform the selected actions 1710 to the ex-vivo brain perfusion device 1712 which, upon receiving the instructions, can execute the instructions to perform the selected actions.

In some implementations, the control system 1700 is physically integrated into the ex-vivo brain perfusion device 1712, i.e., such that the control system 1700 is implemented by computer hardware and software that is physically co-located with the ex-vivo brain perfusion device. In other implementations, the control system 1700 is implemented remotely from the ex-vivo brain perfusion device 1712, e.g., in a remote data center. In these implementations, the sensor measurements 1708 generated by the ex-vivo brain perfusion device 1712 may be transmitted from the ex-vivo brain perfusion device 1712 to the control system 1700 over a data communications network (e.g., the internet). Similarly, the instructions to perform the actions 1710 selected by the control system 1700 can be transmitted from the control system 1700 to the ex-vivo brain perfusion device 1712 over a data communications network. In some implementations, parts of the control system 1700 may be physically integrated into the ex-vivo brain perfusion device 1712, while other parts may be remotely located from the ex-vivo brain perfusion device 1712.

The control system 1700 can include an observation generation engine 1706 and a controller machine learning model 1704, which are each described in more detail next (and throughout this specification).

At each of multiple time steps, i.e., over a period of time during which the control system 1700 controls the ex-vivo brain perfusion device 1712, the observation generation engine 1706 is configured to process current sensor measurements 1708 received from the ex-vivo brain perfusion device 1712 at the time step to generate a current observation 1702 for the time step. The current observation 1702 is an ordered collection of numerical values, e.g., a vector, array, or other tensor of numerical values, that is derived from the current sensor measurements 1708 and that characterizes the current state of the brain at the time step.

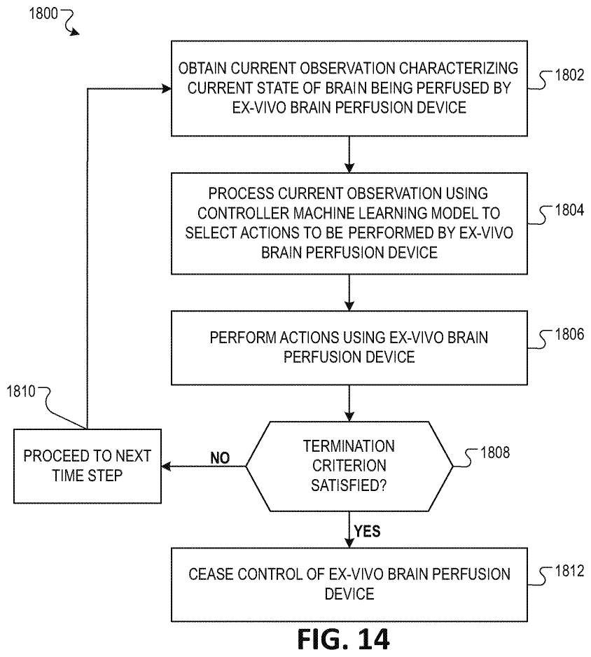

Generally, the current observation 1702 can characterize any appropriate aspects of the current state of the brain, e.g., in relation to rheological features of the perfusate being used to perfuse the brain, or hemodynamic features of the perfusate, or metabolic features of the brain, or operational characteristics of the ex-vivo brain perfusion device. An example process for generating a current observation 1702 characterizing the current state of the brain at a time step is described in more detail with reference to FIG. 14.

At each of multiple time steps, i.e., over a period of time during which the control system 1700 controls the ex-vivo brain perfusion device 1712, the controller machine learning model 1704 receives the current observation 1702 characterizing the current state of the brain at the time step. The current observation 1702 can include, but is not limited to, data received from the pressure sensor 141, the flow sensor 142, the scales 157, 174, 1910, 2117, the camera 1520, the environment sensor 1522, the sensor block 124, the sensor probes 128, the in-line Raman spectroscopy system 107, and/or the sensor module 2107. The controller machine learning model 1704 processes the current observation, in accordance with trained values of a set of controller machine learning model parameters, to generate a model output that defines one or more actions 1710 to be performed by the ex-vivo brain perfusion device 1712. The control system 1700 can then transmit instructions to perform the actions selected by the controller machine learning model 1704 to the ex-vivo brain perfusion device 1712.

The controller machine learning model 1704 can be any appropriate type of machine learning model (e.g., a neural network, or a linear model, or a Gaussian process model, or a combination thereof) and have any appropriate machine learning model architecture. For instance, if the controller machine learning model 1704 is implemented as a neural network, then the controller machine learning model 1704 can include any appropriate types of neural network layers (e.g., fully connected layers, attention layers, convolutional layers, recurrent layers, pooling layers, and so forth) in any appropriate number (e.g., 3 layers, or 10 layers, or 50 layers) and connected in any appropriate configuration (e.g., as a directed graph of layers).

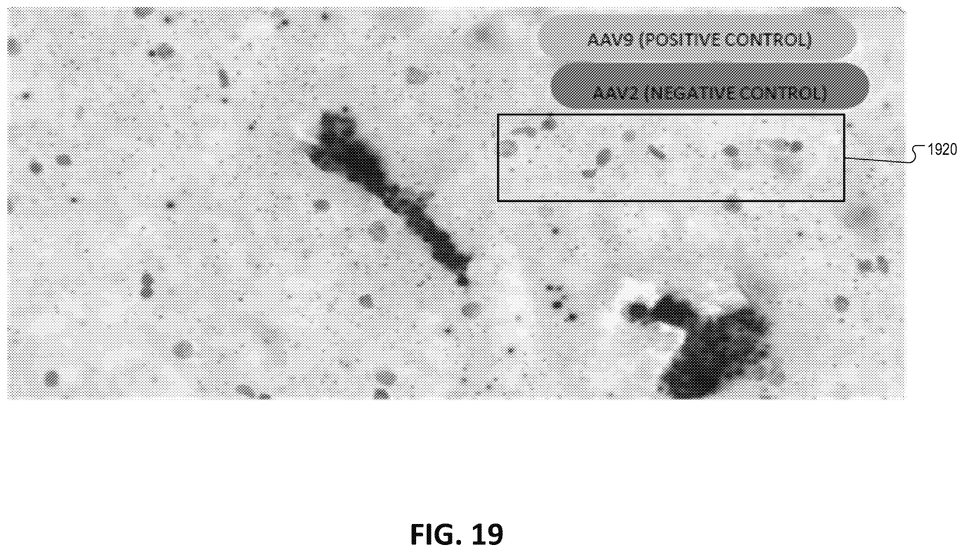

The control system 1700 can train the controller machine learning model 1704 by a machine learning training technique. For instance, control system 1700 can train the controller machine learning model 1704 by a reinforcement learning training technique, e.g., based on rewards that, at each time step, characterize a level of homeostasis of the of the brain. The control system 1700 can thus train the controller machine learning model 1704 to learn to select actions that maintain the brain in a state of homeostasis for an extended period of time, e.g., at least one day, or at least one week, or at least two weeks. An example process for training the controller machine learning model 1704 by a reinforcement learning training technique is described below with reference to FIG. 19.

The control system 1700 can enable sustained, fully autonomous perfusion of ex-vivo brain tissue by learning to dynamically regulate operations of the ex-vivo brain perfusion device 1712 in a manner that reflects the vascular physics and metabolic feedback loops controlling in vivo cerebral circulation. In more detail, in-vivo cerebral circulation is a highly complex process where cerebrovascular endothelial and mural cells transduce variations in wall shear stress, pulsatility, and apparent viscosity at successive arterial bifurcations and within the capillary network. Deviations from physiologically appropriate hemorheological gradients, which are characterized by higher effective viscosity and shear in large vessels and markedly lower values after shear-thinning in the microvasculature, lead to maladaptive vasomotor responses, perturb mass-transfer of oxygen and glucose, and induce transcriptional reprogramming. Concomitantly, ongoing consumption of metabolic substrates and byproduct efflux drive time-dependent changes in perfusate viscosity, density, gas solubility, and electrolyte composition, which in turn drive responses in cerebrovascular endothelial cells.

The control system 1700 addresses these complex dynamics and enables automated control of brain perfusion. In particular, the control system 1700 can leverage real-time sensor measurements generated by sensors of the ex-vivo brain perfusion device and that characterize, e.g., rheological features of the perfusate being used to perfuse the brain, or hemodynamic features of the perfusate, or metabolic features of the brain. The control system 1700 trains the controller machine learning model 1704 to leverage features derived from the real-time sensor measurements to learn to an automated control policy that can preserve cerebral homeostasis and enable extended brain perfusion durations.

In some implementations, the machine learning model 1704 is trained to validate hemodynamic alignment of perfusate during a perfusion process. In some implementations, the machine learning model 1704 predicts hemodynamic features of the perfusate using a pressure-flow (ΔP-Q) model having a root mean square error (RMSE) of 10% or less over a 6 hour perfusion. In some implementations, the pressure-flow (ΔP-Q) model for modelling hemodynamic features of the perfusate has a root mean square error (RMSE) of 5% or less when comparing predictions generated based on sensor data obtained during perfusion versus pressure-flow measurements obtained during catheter-based testing.

FIG. 14 is a flow diagram of an example process 1800 for controlling an ex-vivo brain perfusion device using a controller machine learning model. For convenience, the process 1800 will be described as being performed by a system of one or more computers located in one or more locations. For example, a control system, e.g., the control system 1700 of FIG. 13, appropriately programmed in accordance with this specification, can perform the process 1800.

The system can perform steps 1802-1806 at each time step over a sequence of time steps. For convenience, the description which follows will reference a “current” time step, which can be any time step in the sequence of time steps.

The system obtains a current observation characterizing at least a current state of the brain being perfused by the ex-vivo brain perfusion device (1802). The system can generate the current observation based on sensor measurements generated by one or more sensors of the ex-vivo brain perfusion device(e.g., based on data generated by one or more of the sensor block 124, the sensor probes 128 along the sensor line 1124, the Raman spectroscopy system 107, and the sensor module 2104).

In some implementations, the current observation characterizes at least a current mechanical state of perfusate being used by the ex-vivo brain perfusion device to perfuse the brain. For instance, the current observation can characterize one or more of: a current apparent viscosity of the perfusate; or a current convective-diffusive flux of the perfusate; or a current arterial inflow waveform of the perfusate; or a current flow resistance of the perfusate; or a current pressure of the perfusate; or a current rate of flow of the perfusate.

In some implementations, the current observation characterizes at least a current hemodynamic state of the brain. For instance, the current observation can characterize one or more of: a pressure of the flow of perfusate into the brain; or a flow rate of the flow of perfusate into the brain; or a conductance of perfusate flow through the brain; or an instantaneous multimetric vascular-tone parameters, or a segmental resistance of the brain, or a shear-stress distribution within the brain.

In some implementations, the current observation characterizes at least a current metabolic state of the brain. For instance, the current observation can characterize one or more of: concentrations of metabolites in the perfusate (e.g., one or more of: glucose, lactate, pyruvate, or glutamate), or oxygen partial pressure in the perfusate, or carbon dioxide partial pressure in the perfusate, or a cerebral metabolic rate of oxygen, or a cerebral metabolic rate of glucose, or oxygen-glucose indices, or lactate-glucose indices; or a Raman-derived vector that summarizes the metabolic state of the brain. In some implementations, the system is configured to update the characterization of oxygen-glucose indices and lactate-glucose indices at a rate of 0.1 Hz or less.

The system can utilize the current observation characterizing the current state of the brain to adjust one or more parameters of the system. For example, the system can utilize the current observation characterizing the current state of the brain to adjust one or more parameters of the system to dynamically adjust pump waveforms and perfusate composition in real-time to maintain homeostasis of the brain being perfused by the system.

The system can include some or all of the raw sensor measurements directly in the current observation. As an alternative or in combination, the system can process the raw sensor measurements to generate higher-level integrated metrics for inclusion in the current observation. A few examples of how the system can process the raw sensor data to generate integrated metrics for inclusion in the current observation are described next.

In one example, the system can process sensor measurements characterizing a shear rate of the perfusate (optionally, along with other parameters such as temperature of the perfusate and oxygen level in the perfusate) using a pre-calibrated model to generate a predicted current apparent viscosity (μ) of the perfusate. The model can be, e.g., a Carreau-Yasuda model augmented with Arrhenius temperature and Bunsen gas-solubility terms, and the model can be calibrated based on experimental data across parameter sweeps, e.g., shear rates ranging from 0.1-3000 s⁻¹, temperatures of 20-38 °C, variable O₂/CO₂ partial pressures, and ±20 % compositional changes of the perfusate. In some implementations, the model for predicted apparent viscosity (μ) is a Carreau-Yasuda model with a mean absolute error less than or equal to 5%. In some implementations, the model for predicted apparent viscosity (μ) is validated using a 20% hold out test data set. In some implementations, the system is calibrated to generate a single shear rate (100^{s-1}) repeatability with a coefficient of variability (CV) less than or equal to 5% at 37 °C.

The apparent viscosity of the perfusate can capture metabolic thickening phenomena—such as CO₂-induced pH shifts or protein leakage—that elevate hydraulic resistance. After generating the predicted current apparent viscosity of the perfusate based using the sensor measurements and the pre-calibrated model, the system can include the predicted current apparent viscosity in the current observation. In some implementations, the apparent viscosity (μ) is mapped over a 4-D grid of shear-rate, temperature, pO₂, and variations in solute concentration.

As another example, the system can process sensor measurements including high-resolution micro-particle image velocimetry (μPIV) of the arterial cannula to generate features characterizing convective-diffusive substrate flux, e.g., in the form of Sherwood boundary limits that subsequent runs must maintain to preserve convective-diffusive substrate flux within blood-like bounds. In some implementations, high-resolution micro-particle image velocimetry (μPIV) of the arterial cannula can be performed for each perfusate formulation and hardware configuration of the system, under physiological pulsatility, to establish a reference velocity field V(r, t) and wall-shear profile τ_w(t), which can then be used as boundary limits for mass-transfer modeling. In some implementations, the convective-diffusive substrate flux modeled by the system can be used to adaptively control and modify pump waveforms and perfusate composition throughout perfusion (e.g., every few seconds) in order to maintain cerebral homeostasis of the perfused brain.

As another example, the system can process sensor measurements generated by paired arterial- and venous-line blood-gas analyzers (e.g., sensor 128 of FIG. 1), e.g., including O₂ and CO₂ partial pressures, glucose and lactate concentrations, and pH. The system can apply a model based on the Fick principle to compute cerebral metabolic rates of oxygen and glucose (CMRO₂, CMR-Glc), oxygen-glucose and lactate-glucose indices (OGI, LGI). These metrics provide a real-time quantitative budget of oxidative versus glycolytic energy consumption, and the system can include them in the current observation. In some implementations, the perfusion system is configured to maintain the cerebral metabolic rate of oxygen (CMRO₂) within ±10% of the CMRO₂ value measured 3 hours after equilibration of the system 100 for a perfusion period of 22 hours or longer. In some implementations, the perfusion system is configured to maintain the cerebral metabolic rate of oxygen (CMRO₂) within ±10% of the CMRO₂ value measured 1 hour after equilibration of the system 100 for 90% or more of a 6 hour perfusion. In some implementations, the system is configured to maintain perfusion pressure and CMRO₂ values within a target range for 95% or more of an entire perfusion process.

As another example, the system can process sensor measurements generated by a Raman spectroscopy probe positioned in-line with the venous effluent (e.g., Raman probe 119 of FIG. 1) to generate features characterizing concentrations of metabolites present in the perfusate, such as glucose, lactate, pyruvate, glutamate, and NADH/NAD⁺ proxies. In some cases, the system can process the Raman spectroscopy sensor measurements to predict (e.g., at a rate of 0.1 Hz) transcriptional, proteomic, and metabolomic states, e.g., relating to hypoxia or oxidative-inflammatory-nitrosative stress. The system can include the features derived from the Raman spectroscopy in the current observation. In some implementations, the system is configured to achieve an F1 score greater than or equal to 0.8 for binary classifiers for hypoxia, oxidative stress, and neuro-inflammation. In some implementations, the system is configured to achieve a factor analysis (FA) greater than or equal to 0.8 for quantized cross-modality prediction based on sensor measurements generated by a Raman spectroscopy probe positioned in-line with the venous effluent (e.g., Raman probe 119 of FIG. 1). In some implementations, the system is configured to process the Raman spectroscopy sensor measurements to generate a molecular vector state that can be used to infer metabolite levels, neuropeptide surges (e.g., vasopressin), and transcriptional signatures at a rate of 0.1 Hz.

The system can process the current observation characterizing the current state of the brain using the controller machine learning model to generate an output that defines one or more actions to be performed by the ex-vivo brain perfusion device (1804). For instance, the controller machine learning model can process the current observation to generate a score distribution over a set of possible actions. The system can then select one or more actions to be performed by the ex-vivo brain perfusion device based on the score distribution. For instance, the system can select one or more actions having the highest scores under the score distribution. As another example, the system can sample one or more actions from a probability distribution over the set of possible actions that is defined based on the score distribution, e.g., by processing the score distribution using a soft-max function.

In some implementations, the set of possible actions can include actions that modify one or more chemical parameters of the perfusate. For instance, the set of possible actions can include actions that modify one or more of: a chemical composition of the perfusate, or a pH of the perfusate, or a viscosity of the perfusate. Modifying the chemical composition of the perfusate can include, e.g., increasing or decreasing the concentrations of particular metabolites (e.g., buffers or electrolytes) in the perfusate using (i) the syringe pump manifolds 167, 1167, (ii) the supplementary pump 1906, and/or (iii) the dialysis filtration system 109.

In some implementations, the set of possible actions can include actions that modify one or more operational parameters of the ex-vivo brain perfusion device. For instance, the set of possible actions can include actions that modify one or more of: a flow waveform of the perfusate, or a degassing rate of the perfusate, or a pressure applied to the perfusate, for example, through control of the pulse generator 136.

A physics-regularized neural surrogate of the multiscale flow model provides differentiable Jacobians ∂y/∂a that link potential actuator moves (e.g., hardware setting control and/or adjustments of the perfusate chemical composition) to predicted shifts in both mechanical and metabolic outputs. For example, one or more of pump waveforms, inlet pressure limits, degassing rates, substrate infusion and buffer infusion can be mapped to predicted outcomes, such as ΔP, shear stress, CMRO₂, lactate, and/or oxygen-glucose indices. In some examples, the multiscale flow model is configured to predict arterial pressure and flow within the system with a mean absolute error less than or equal to 5 mm Hg/ 5 mL min⁻¹ (with a 5-fold coefficient of variability). Physics regularization enforces mass-balance and Navier-Stokes constraints.

The system causes the ex-vivo brain perfusion device to perform the one or more actions selected by the controller machine learning model (1806). For instance, the system can transmit instructions to one or more control modules or actuators of the ex-vivo brain perfusion device to implement the one or more actions selected by the controller machine learning model. For instance, to implement an action to increase the concentration of a metabolite in the perfusate, the system can transmit an instruction to an actuator (e.g., a syringe pump of one of the syringe pump manifold 167, 1167 of FIG. 1) to inject a higher quantity of the metabolite into the perfusate that is passed through the brain (e.g., into the perfusate in the perfusate reservoir 104 of FIG. 1). As another example, to implement an action to modify a flow waveform of the perfusate, the system can transmit an instruction to modify operation of a pumping device (e.g., pulse generator 136 of FIG. 1) that is used to pump the perfusate through the brain.

The system determines whether a termination criterion is satisfied (1808). In one example, the termination criterion can be that the system has controlled the ex vivo brain perfusion device over at least a predefined duration of time. In another example, the termination criterion can be that the brain is in a state of homeostatic balance or regulation, as determined based on sensor measurements obtained from the ex-vivo brain perfusion device. In another example, the termination criterion may be that a user of the system provides a command to cease control of the ex-vivo brain perfusion device, e.g., by way of a user interface made available by the system.

By continuously measuring rheological properties of the perfusate in real-time along with arterial and venous pressure, flow, and exchange recordings, the system can infer hidden cerebrovascular states (e.g., dynamic cerebral resistance and cerebral tone) that govern mass transfer and metabolic extraction in the brain, thereby transforming raw sensor data into actionable physiological metrics for closed-loop control to optimize brain function during perfusion. In some implementations, the system is trained to determine an instantaneous global cerebrovascular resistance of a perfused brain at time t throughout the perfusion. In some implementations, the system is trained to provide a brain cerebrovascular state model that predicts resistance of the perfused brain at time t+n with a root mean square error of 10% or less and generates a cerebrovascular state vector.

In response to determining that the termination criterion is satisfied, the system ceases control of the ex-vivo brain perfusion device (1812).

In response to determining that the termination criterion is not satisfied, the system proceeds to the next time step (1810) and returns to step 1802.

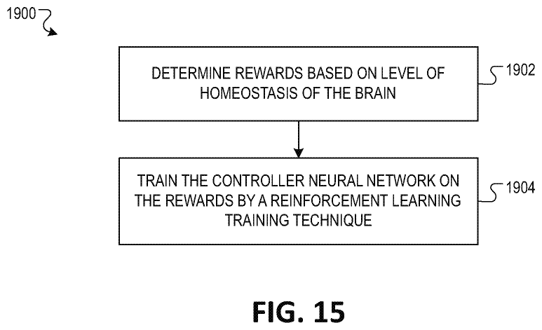

FIG. 15 is a flow diagram of an example process 1900 for training a controller machine learning model used to control an ex-vivo brain perfusion device. For convenience, the process 1900 will be described as being performed by a system of one or more computers located in one or more locations. For example, a control system, e.g., the control system 1700 of FIG. 13, appropriately programmed in accordance with this specification, can perform the process 1900.

The system determines a reward (which can be represented, e.g., as a scalar numerical value) for each time step in a sequence of time steps during which the ex vivo brain perfusion device is perfusing a brain (1902). The reward for a time step can characterize a level of homeostasis of the brain at the time step.

The sequence of time steps over which the rewards are computed can be a sequence of time steps where the ex vivo brain perfusion device was controlled by the controller machine learning model, but does not have to be. For instance, the ex vivo brain perfusion device may have been controlled by a heuristic, rules-based controller over the sequence of time steps, or may have been manually controller by a human operator.

In some implementations, the system determines the reward for a time step based at least in part on a discrepancy between: (i) the current metabolic state of the brain at the time step, and (ii) a target metabolic state of the brain. The target metabolic state of the brain can be a metabolic state when the brain is in metabolic homeostasis, such that reward for a time step characterizes a degree to which the brain is in metabolic homeostasis at the time step. The system can determine the current metabolic state of the brain based on current sensor measurements, e.g., sensor measurements generated by Raman spectroscopy of perfusate that is being used to perfuse the brain. The system can represent the current and target metabolic states of the brain as respective tensors (e.g., vectors or arrays) or numerical values, and can measure a discrepancy between the current and target metabolic states, e.g., using an L1 norm or an L2 norm.

In some implementations, the system determines the reward for a time step based at least in part on a discrepancy between: (i) a current mechanical state of the perfusate at the time step, and (ii) a target mechanical state of the perfusate. The target mechanical state of the perfusate can be a mechanical state when the brain is in mechanical homeostasis, such that the reward for a time step characterizes a degree to which the brain is in mechanical homeostasis at the time step. The system can determine the current mechanical state of the perfusate at a time step based on current sensor measurements, e.g., that characterize one or more of: current apparent viscosity of the perfusate; current convective-diffusive flux of the perfusate; or a current arterial inflow waveform of the perfusate. The system can represent the current and target mechanical states of the brain as respective tensors (e.g., vectors or arrays) or numerical values, and can measure a discrepancy between the current and target mechanical states, e.g., using an L1 norm or an L2 norm.

In some implementations, the system determines the reward for a time step based on both: (i) a discrepancy between current and target metabolic states of the brain, and (ii) a discrepancy between current and target mechanical states of the brain. For instance, the system can determine the overall reward as a weighted linear combination of respective rewards characterizing metabolic and mechanical homeostasis of the brain.

The system trains the training the controller machine learning model on the rewards for the sequence of time steps using a reinforcement learning training technique (1904). In particular, the system trains the controller machine learning model to increase a cumulative measure of rewards received by controlling the ex-vivo brain perfusion device using the controller machine learning model. The reinforcement learning technique can be any appropriate reinforcement learning technique, e.g., an actor-critic reinforcement learning technique or a policy gradient reinforcement learning technique. The rewards characterize a level of homeostasis of the brain, and therefore training the controller machine learning model on the rewards causes the controller machine learning model to learn an action selection policy that encourages the brain to maintain homeostasis.

The system can train the controller machine learning model on rewards corresponding to multiple sequences of time steps, e.g., corresponding to multiple “runs” of the ex vivo brain perfusion device. For instance, the system can train the controller neural network on rewards from at least 100, or at least 1000, or at least 10,000 sequences of time steps over which ex-vivo brain perfusion device perfuses respective brains. Each sequence of time steps can include, e.g., at least 100, or at least 1000, or at least 10,000 time steps. The time steps themselves can be separated by any appropriate duration of time, e.g., 0.1 seconds, or 1 second, or 10 seconds.

Training the controller machine learning model can refer to adapting the parameter values of the controller machine learning model to optimize a reinforcement learning objective function. The specific nature of the training can depend on the specific type and architecture of the machine learning model. For instance, for a controller machine learning model implemented as a neural network, training the controller machine learning model can include backpropagating gradients of a reinforcement learning objective function through the neural network.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

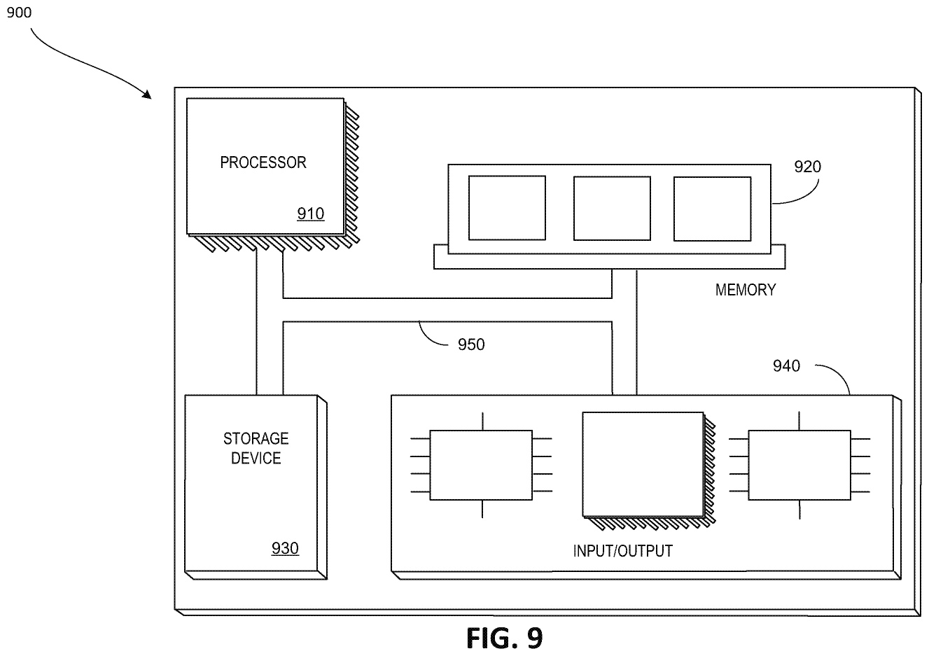

FIG. 9 is a block diagram of an example computer system 900. For example, referring to FIG. 1, the computer system 188 of the perfusate system 100 could be an example of the system 900 described here. The system 900 includes a processor 910, a memory 920, a storage device 930, and an input/output interface 940. Each of the components 910, 920, 930, and 940 can be interconnected, for example, using a system bus 950. The processor 910 is capable of processing instructions for execution within the system 900. The processor 910 can be a single-threaded processor, a multi-threaded processor, or a quantum computer. The processor 910 is capable of processing instructions stored in the memory 920 or on the storage device 930. The processor 910 may execute operations such as receiving signals from one or more sensors (e.g., the pressure sensors 140, 141, 164, 168, 195, 1008, the flow sensors 130, 142, and the temperature sensor 121 shown in FIG. 1) and controlling or more elements of the system (e.g., the solenoid valves 133, 147, 149, 151, 158, 171, 173, 178, 1120, 1121 of FIG. 1) based on the received signals. In some implementations, all of the real-time sensors within the system 100 (e.g., the pressure sensors 140, 141, 164, 168, 195, 1008, the flow sensors 130, 142, and the temperature sensor 121 shown in FIG. 1) are synchronized with one another such that the time-skew stamp between any two sensor channels is less than 100 ms during a 10 minute recording period. In some implementations, missing or corrupt packets of data are received from one or more the sensors at a rate of less than 1 per 10,000 packets (i.e., less than 0.01%) during a two hour perfusion period.

The memory 920 stores information within the system 900. In some implementations, the memory 920 is a computer-readable medium. The memory 920 can, for example, be a volatile memory unit or a non-volatile memory unit. In some implementations, the memory 920 stores a data structure. In some implementations, multiple data structures are used.

The storage device 930 is capable of providing mass storage for the system 900. In some implementations, the storage device 930 is a non-transitory computer-readable medium. The storage device 930 can include, for example, a hard disk device, an optical disk device, a solid-date drive, a flash drive, magnetic tape, or some other large capacity storage device. The storage device 930 may alternatively be a cloud storage device, e.g., a logical storage device including multiple physical storage devices distributed on a network and accessed using a network.

The input/output interface 940 provides input/output operations for the system 900. In some implementations, the input/output interface 940 includes one or more of network interface devices (e.g., an Ethernet card), a serial communication device (e.g., an RS-232 10 port), and/or a wireless interface device (e.g., an 802.11 card, a 3G wireless modem, or a 4G wireless modem). In some implementations, the input/output device includes driver devices configured to receive input data and send output data to other input/output devices, e.g., keyboard, printer and display devices (e.g., display device 189 of FIG. 1). In some implementations, mobile computing devices, mobile communication devices, and other devices are used.

In some implementations, the system 900 is a microcontroller. A microcontroller is a device that contains multiple elements of a computer system in a single electronics package. For example, the single electronics package could contain the processor 910, the memory 920, the storage device 930, and input/output interfaces 940.

Although an example processing system has been described in FIG. 9, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine readable storage device, a machine readable storage substrate, a memory device, a composition of matter effecting a machine readable propagated signal, or a combination of one or more of them.

The term “computer system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks and CD-ROM and DVD-ROM disks. The processor and the memory can be supplemented by, or incorporated in, special purpose logic circuitry. The components of the system can be interconnected by any form or medium of digital data communication, e.g., a communication network. Examples of communication networks include a local area network (“LAN”) and a wide area network (“WAN”), e.g., the Internet.

A number of embodiments have been described. Nevertheless, it will be understood that various modifications may be made without departing from the spirit and scope of the disclosure.

For example, while the perfusate system 100 has been described as including both a filter bag 101 inside the perfusate reservoir 104 and an in-line mechanical filtration system 1001 has been described as including a filtration manifold 1114, in some implementations, the perfusate system 100 includes only the filter bag 101 inside the perfusate the reservoir 104 to filter the perfusate entering the reservoir 104 without the in-line mechanical filtration system 1001. In other implementations, the perfusate system 100 includes only the in-line mechanical filtration system 1001 to filter the perfusate entering the reservoir 104 without the filter bag 101 inside the perfusate the reservoir 104.

In addition, while the pumps 111, 114, 184, 190, 1002, 1122, 1906, 2108 have been described as being peristaltic pumps, in some implementations, other types of pumps can be used. In some implementations, one or more of the pumps 111, 114, 184, 190, 1002, 1122, 1906, 2108 are roller pumps or centrifugal pumps.

While the brain housing 102 has been described as having a knob 1018 for handling the lid 1026, in some implementations, the brain housing 102 includes a pair of handles to enable removal of the lid 1026 from the basin 1022.

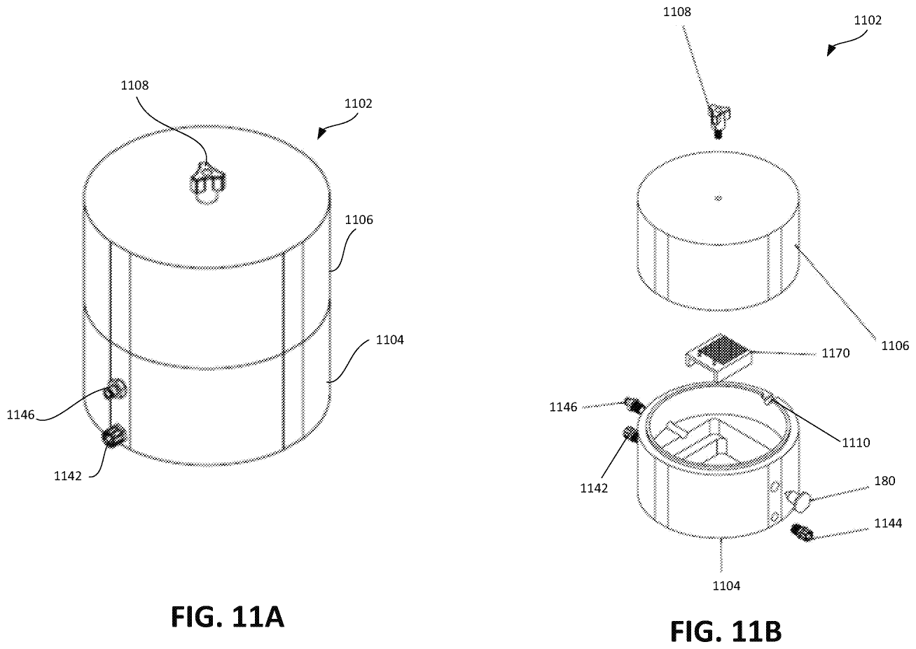

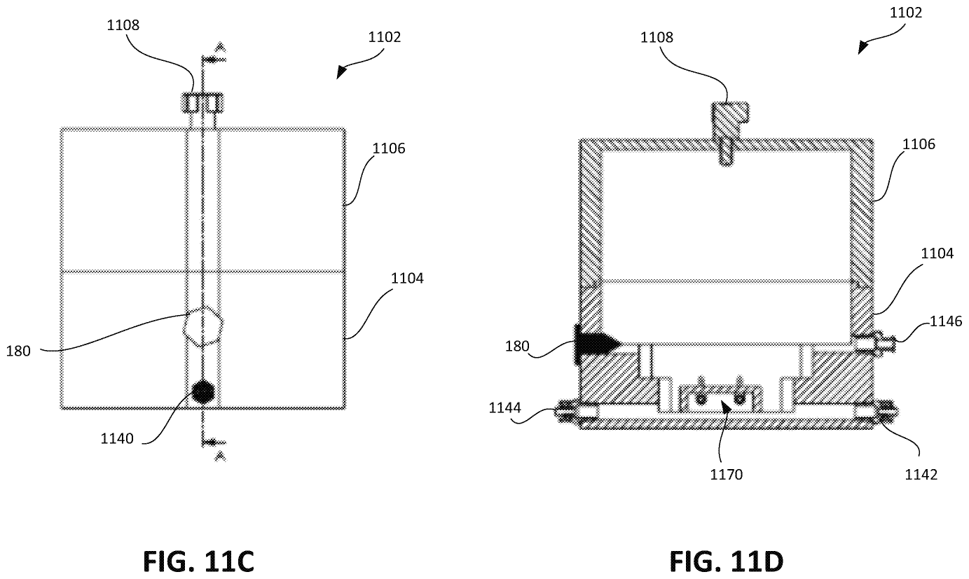

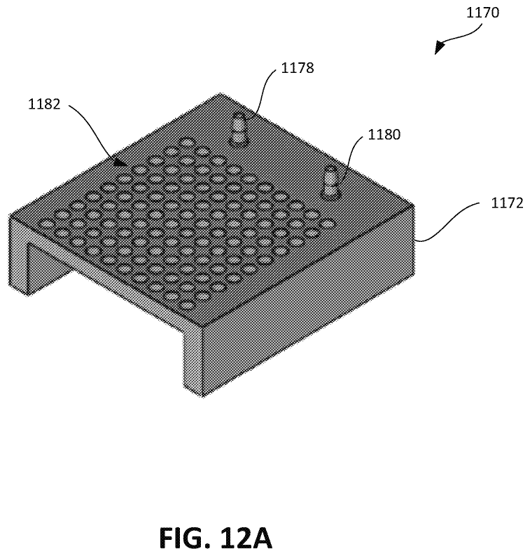

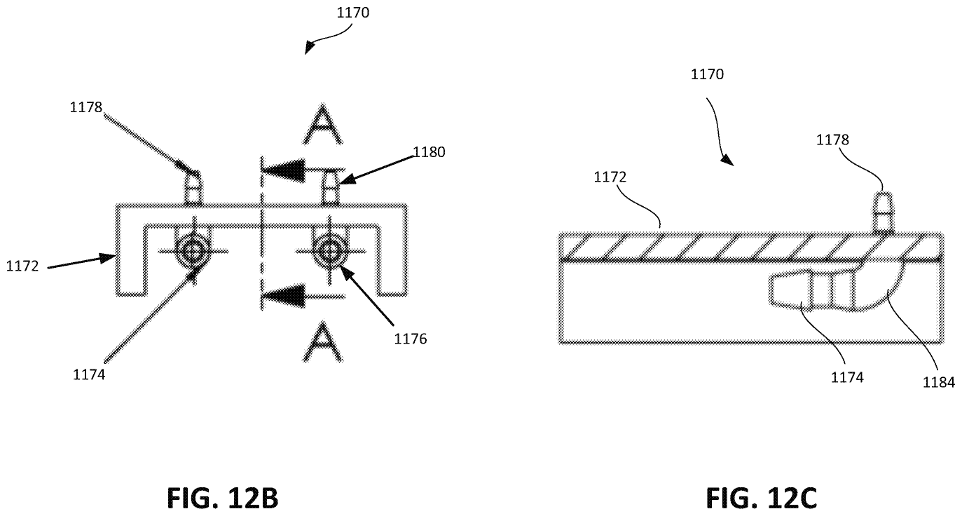

While the brain 110 has been described as being fluidly coupled to the organ line 144 using pig vasculature, in some implementations, the organ line 144 is fluidly coupled to the brain 110 positioned in the housing through a brain interface 1170 inside the brain housing 102, as depicted in FIGS. 11B, 11D, and 12A-12C. FIGS. 12A-12C depict an example brain interface 1170 for fluidly coupling a brain to the organ line 144. The brain interface 1170 is a 3D-printed mechanical interface that includes a base plate configured to stabilize and support the brain 110 inside the housing, two fluid inlets, and two corresponding fluid outlets. A y-connector is coupled to an end of the organ line 144, and each of the fluid inlets of the brain interface 1170 are fluidly coupled to the organ line 144 using the y-connector and each inlet of the brain interface 1170 receives fresh perfusate flowing out of the organ line 144 into the brain interface 1170. The brain interface 1170 defines flow paths fluidly coupling each of the fluid inlets of the brain interface 1170 to a respective outlet of the brain interface 1170. Prior to placing the brain 110 in the brain housing 102 and beginning perfusion of the brain 110, each of the carotid arteries of the brain 110 is sutured to a respective fluid outlet of the brain interface 1170. Once the carotid arteries of the brain 110 are sutured to the fluid outlets of the brain interface 1170, the inlets of the brain interface 1170 are coupled to the organ line 144 using a y-connector and the brain 110 and brain interface 1170 are enclosed in the brain housing 102 throughout the perfusion process. As fresh perfusate flows along the organ line 144, the perfusate enters the fluid inlets of the brain interface 1170 and flows through the fluid pathways defined by the brain interface 1170, through the fluid outlets of the brain interface 1170, and into the carotid arteries of the brain 110.

While the brain interface 1170 has been described as including two fluid outlets with each of the carotid arteries of the brain 110 being sutured to a respective fluid outlet of the brain interface 1170, in some implementations, the brain interface 1170 includes other numbers of fluid outlets (e.g., 1, 3, 4, etc.). For example, the brain interface 1170 can include a single fluid outlet and a single carotid artery of the brain 110 can be coupled to the fluid outlet of the brain interface 1170.

In addition, while the brain interface 1170 has been described as including two fluid inlets, in some implementations, the brain interface 1170 includes a single fluid inlet that can be coupled to the organ line 144 and that is fluidly coupled to the two fluid outlets of the brain interface 1170.

While the brain housing stand 1112 has been described as having four actuators 1730, 1732, 1734, 1736, in some implementations, the brain housing stand 1112 has other numbers of actuators (e.g., 1, 2, 3, 5, 6, etc.).

In addition, while the linear actuators 1720, 1722, 1724, 1726 of the brain housing stand 1112 have been described as being actuated in sequence at a preset rate and in a predetermined order, in some implementations, the linear actuators 1720, 1722, 1724, 1726 of the brain housing stand 1112 are actuated dynamically based on sensor data. For example, sensor data collected by the system 100 indicating the health of the brain 110 (e.g., sensor data collected by the camera 1520, colorimeter 1116, photometers 1117, 1118, fluorimeter 1119, etc.) can be provided to a trained machine learning model to determine the health of the brain 110 in real time during perfusion and, based on the determination, the machine learning model can generate control commands to actuate one or more linear actuators 1720, 1722, 1724, 1726 to tilt the brain housing 102 to improve the health of the brain tissue and potentially extend the length of perfusion of the brain 110.

While the level sensors 125, 180 have been described as being infrared level sensor, other types of level sensors can be used to detect the level of perfusate in the sensor block 124 and the brain housing 102, respectively. For example, the level sensors 125, 180 are ultrasonic sensors, capacitance sensors, or other types of optical sensors.

While the sensor block inlet line 146 has been described as a y-splitter line with a single line flowing out of the pulse generator 136 that splits into two fluid lines flowing into the sensor block 124, in other implementations, the sensor block inlet line can be a single, unsplit line.

While the dialysis filtration system 109 has been described as filtering the perfusate without the use of an exchange solution, in some implementations, the dialysis filtration system 109 includes a source of exchange solution and the perfusate is filtered using the exchange solution. For example, in some implementations, one or more dialyzers 1191, 1192, 1193 of the dialyzer manifold 191 is a capillary dialyzer, and perfusate and exchange solution are pumped through the dialyzers 1191, 1192, 1193 at the same time. The exchange solution flowing through the dialyzers 1191, 1192, 1193 interacts with the perfusate flowing through the dialyzers 1191, 1192, 1193 to remove waste products from the perfusate and add nutrients to the perfusate. For example, while the perfusate flows within porous microtubes of the dialyzers 1191, 1192, 1193, the exchange solution can flow outside the porous microtubes such that waste products pass from the perfusate in the interior of the porous microtubes to the exchange solution surrounding the porous microtubes and such that nutrients pass from the exchange solution surrounding the porous microtubes into the persuade inside the microtubes. The filtered perfusate exits the dialyzer manifold 191 and flows back to the perfusate reservoir 104 along the dialyzer outlet line 193, while the exchange solution exits the dialyzer manifold 191 and flows to the dialysis solution receptacle 117 via the waste line 197. In some embodiments, the exchange solution is replaced or replenished over time to prevent the depletion of nutrients or the accumulation of toxins.

While the perfusion system 100 has been described as using the sensor pump to pump perfusate from the brain housing 102 to the perfusate reservoir 104, in some implementations, the perfusion system includes an additional pump on the housing outlet line 176 that can be used to pump perfusate from the brain housing 102 to the perfusate reservoir 104. For example, in response to the level sensor 180 generating a signal indicating that the fluid level inside the brain housing 102 has exceeded a threshold fluid level, the solenoid valve 178 is opened and a peristaltic positioned along the housing outlet line 176 is operated to pump used perfusate from the brain housing 102 along the housing outlet line 176 to the perfusate reservoir 104. Once the level sensor 180 detects that the fluid level inside the brain housing 102 has fallen below the threshold level, the solenoid valves 178 is closed, which prevents fluid from flowing from the brain housing 102 to the perfusate reservoir 104.

While the perfusion system 100 has been described as including two syringe pump manifolds 167 with two or more syringes filled with pharmaceutical and/or chemical compound, in some implementations, the perfusion system 100 includes a single syringe pump with a single syringe filled with a compound. In addition, in some implementations, the perfusion system 100 includes a single syringe pump manifold.

While the lid 218, the central housing 202, and the base 220 of the pulse generator 136 have been described as being attached to one another using a set of bolts 224 and nuts 228, other types of mechanical fasteners can be used to couple the lid 218, central housing 202, and base 220. For example, in some implementations, the lid 218, the central housing 202, and the base 220 are attached to one another using one or more of bolts, rivets, pins, or adhesive.

While the drain line 143 of the perfusate system 100 has been described as being coupled to a waste container 145 to collect the perfusate drained from the system 100 along the drain line 143, in some implementations, the drain line 143 is directly coupled a drain and perfusate flowing along the drain line 143 is directed into the drain coupled to the drain line 143.

While the stopcocks 127, 132, 135, 155, 159, 181, 182, 1109, 1111, 1113, 1115, 1169, 1171, 1175, 1181, 1183, 1188, 1189, 1194, 1195, 1196, 1197, 1198, 1199 of the system have been described as being manually operated by a user of the system 100 to collect perfusate samples, in some implementations, one or more of the stopcocks 127, 132, 135, 155, 159, 181, 182, 1109, 1111, 1113, 1115, 1169, 1171, 1175, 1181, 1183, 1188, 1189, 1194, 1195, 1196, 1197, 1198, 1199 can be automatically controlled to collect samples of the perfusate. For example, one or more of the stopcocks 127, 132, 135, 155, 159, 181, 182, 1109, 1111, 1113, 1115, 1169, 1171, 1175, 1181, 1183, 1188, 1189, 1194, 1195, 1196, 1197, 1198, 1199 can be automatically controlled to collect a sample of perfusate from the system 100 at one or more particular points during the perfusion process. For example, one or more of the stopcocks 127, 132, 135, 155, 159, 181, 182, 1109, 1111, 1113, 1115, 1169, 1171, 1175, 1181, 1183, 1188, 1189, 1194, 1195, 1196, 1197, 1198, 1199 can be fluidly coupled to an autosampler or a sensor (e.g., pressure sensor) in order to automatically collect fluid sample or other data using the respective stopcocks 127, 132, 135, 155, 159, 181, 182, 1109, 1111, 1113, 1115, 1169, 1171, 1175, 1181, 1183, 1188, 1189, 1194, 1195, 1196, 1197, 1198, 1199. In addition, the stopcocks 127, 132, 135, 155, 159, 181, 182, 1109, 1111, 1113, 1115, 1169, 1171, 1175, 1181, 1183, 1188, 1189, 1194, 1195, 1196, 1197, 1198, 1199, 181, 182 have previously been described as automatically being controlled using servo-motor to control and change flow paths throughout the system 100. However, in some implementations, an operator can manually operate one or more of the stopcocks 127, 132, 135, 155, 159, 181, 182, 1109, 1111, 1113, 1115, 1169, 1171, 1175, 1181, 1183, 1188, 1189, 1194, 1195, 1196, 1197, 1198, 1199 to alter the flow paths of the system.

While the computer system 188 has been depicted as including a single computing device, in some implementations, the computing system 188 includes multiple computing devices or processors (2, 3, 4, etc.) in communication with the various electronic components of the perfusion system 100 and configured to control operation of one or more electronic components of the perfusion system 100. Each of the computing devices of the computer system 188 may be in wired or wireless communication with one or more electronic components of the perfusion system 100. In addition, one or more of the computing devices of the computer system 188 can be a remote computing device that is located at a different, remote location from the perfusion system 188.

In addition, while the process of performing an initial flush of the brain 110 using the perfusate system 100 has been described as being stopped automatically in response to the waste scale 157 detecting that a threshold weight of perfusate has been received by the waste container 145, in some implementations, the initial flush procedure is manually stopped by a user. For example, in some implementations, the waste container 145 includes one or more markings indicating the volume of fluid inside the waste container 145, and as perfusate flows along the drain line 143 into the waste container 145, an operator of the system 100 monitors the volume of perfusate collected by the waste container 145. In response to the operator observing that the waste container 145 has received a threshold volume of perfusate, the operator closes the solenoid valve 147 along the drain line 143. In some implementations, the operator adjusts the solenoid valve, 177 to stop the initial flush of the brain 110 in response to observing that 500 mL of perfusate has been collected in the waste container 145. In some implementations, the operator of the system 100 adjusts the solenoid valve 177 to stop the initial flush of the brain 110 based on a predetermined amount of time elapsing since the start of the initial flush procedure. In some implementations, for example, an operator of the system 100 manually stops the initial flush procedure 30 minutes to 1 hour after starting the flush procedure.

While the in-line Raman spectroscopy system 107 has been described as having a single temperature sensor 121 downstream of the heat exchanger block 113, in some implementations, the in-line Raman spectroscopy system 107 includes an additional temperature sensor along the sample line 123 upstream of the heat exchanger block 113. The temperature signals generated by the additional temperature sensor positioned upstream of the heat exchanger block 113 can be compared to the temperature sensor 121 downstream of the heat exchanger block 113 and the heat exchanger 115 can be controlled based on the difference between the temperatures detected by the upstream temperature sensor and the downstream temperature sensor 121.

While the perfusion system 100 has been described as an in-line Raman spectroscopy system 107 for performing Raman spectroscopy on perfusate, in some implementations, the perfusion system 100 also includes a Raman spectroscopy probe for performing Raman spectroscopy on the tissue of the brain 110 in the brain housing 102. For example, the perfusion system 100 can also include a Raman spectroscopy probe that is coupled to or positioned near the brain housing 102 for performing Raman spectroscopy on the tissue of the brain 110 in real-time during perfusion of the brain 110. Once the brain 110 is fluidly coupled to the organ line 144 and is positioned inside the brain housing 102, a Raman spectroscopy probe can be positioned on the surface of the brain 110 and can be operated periodically throughout the perfusion process to perform Raman spectroscopy on the tissue of the brain 110 in real-time as the brain is perfused. Referring to FIG. 11A, in some implementations, the brain housing 102 includes a lid 1106 removably coupled to a basin 1104 and, in order to position the Raman spectroscopy probe on or near the surface of the brain 110, a lid 1106 of the brain housing 102 is removed from the basin 1104. In some implementations, the Raman spectroscopy probe is incorporated into the brain housing 102 (e.g., into a lid 1106 of the housing 102) and the housing 102 can remain closed and pressurized while performing Raman spectroscopy on the brain 110 inside the housing 102.

Similar to the Raman spectroscopy data generated for the perfusate by the Raman spectroscopy system 107, the data generated by the Raman spectroscopy probe on the brain 110 can be used to determine the metabolic activity of the brain 110 and chemical compounds generated by the brain 110 in real-time throughout the perfusion process. For example, at the beginning of the perfusion process, the Raman spectroscopy probe positioned on the surface of the brain 110 can be controlled to perform Raman spectroscopy on the brain 110 to identify molecules and chemical compounds present in the tissue of the brain 110 in order to form a baseline spectroscopy reading for the brain tissue. After the brain 110 has been perfused with the perfusate for a predetermined period, the Raman spectroscopy probe positioned on the surface of the brain 110 can be controlled to perform Raman spectroscopy on the brain 110 and the results can be compared to the baseline spectroscopy reading for the brain tissue to determine changes in the types and concentrations of molecules present in the tissue of the brain 110 compared to the baseline. These changes in the molecular composition of the brain tissue in response to perfusion of the brain 110 detected by the Raman spectroscopy probe can be used to determine the effectiveness of the perfusion process, as well as predict the length of time the brain 110 can be effectively perfused, for example, before the brain 110 undergoes significant swelling.

Similar to the Raman spectroscopy system 107, the Raman spectroscopy probe positioned on the surface of the brain 110 can also be used to determine the effect of a pharmaceutical compound on the brain 110. As discussed above, the syringe pump manifold 167 and the dialysis filtration system 109 can be operated to provide a pharmaceutical compound to the brain 110 through the perfusate at a rate corresponding to a particular release profile. As the pharmaceutical compound is simultaneously provided to the brain 110 from the perfusate reservoir 104, the Raman spectroscopy probe positioned on the surface of the brain 110 can be controlled to perform Raman spectroscopy on the tissue of the brain 110 at predetermined intervals as the pharmaceutical compound is provided to the brain 110.

The data generated by the Raman spectroscopy probe positioned on the surface of the brain 110 can be used to determine whether the pharmaceutical compound is capable of crossing the blood-brain barrier. The pharmaceutical compound can be determined as capable of crossing the blood-brain barrier in response to detecting that one or more chemical compounds corresponding to the pharmaceutical compound are present in the tissue of the brain 110 using the Raman spectroscopy probe positioned on the surface of the brain 110. In addition, the percentage of the pharmaceutical compound taken up and metabolized by the brain 110 can be determined based on the concentration of one or more chemical compounds corresponding to the pharmaceutical compound that is detected in the tissue of the brain 110 by the Raman spectroscopy probe positioned on the surface of the brain 110.

In addition, the Raman spectroscopy probe positioned on the surface of the brain 110 can be used to determine one or more chemical compounds generated by the brain 110 in response to administration of the pharmaceutical compound. For example, the Raman spectroscopy probe 172 can be controlled to perform Raman spectroscopy on the tissue of the brain 110 at predetermined intervals as a pharmaceutical compound is provided to the brain 110 through the perfusate. The results can be compared to a spectroscopy analysis of the tissue of the brain 110 taken prior to introduction of the pharmaceutical compound to the perfusate in order to determine changes in the types and concentrations of molecules present in the tissue of the brain 110 resulting from the introduction of the pharmaceutical compound. These changes in the molecular composition of the brain tissue in response to introduction of the pharmaceutical compound that are detected by the Raman spectroscopy probe can be used to determine the reaction of the brain 110 to the pharmaceutical compound. For example, the molecules produced by the brain 110 in response to the introduction of the pharmaceutical compound into the perfusate and detected in the brain tissue by the Raman spectroscopy probe positioned on the surface of the brain 110 can be used to determine the reaction of the brain 110 to the pharmaceutical compound, the likely efficacy of the pharmaceutical compound on treating the targeted disease, and potential side effects that may result from the pharmaceutical compound. As a result, potential new drugs can be tested using the perfusate system 100 to accurately determine their likely effectiveness in treating the target disease in humans.

Using a Raman spectroscopy probe positioned on the surface of the brain 110 and the Raman spectroscopy system 107 to take Raman spectroscopy measurements on the tissue of the brain 110 and the used perfusate flowing out of the brain 110, respectively, in real-time throughout the perfusion process allows for real-time analysis of the chemical processes occurring in the brain 110 during perfusion without requiring samples of the brain tissue to be removed during the perfusion process. As a result, the sterility and functionality of the brain 110 can be more closely regulated and maintained, which improves and prolongs the perfusion process.

In addition, the data generated by performing Raman spectroscopy on the brain tissue using a Raman spectroscopy probe positioned on the surface of the brain 110 and on the used perfusate using the Raman spectroscopy system 107 can be provided to a trained machine learning model in order to predict one or more characteristics about the brain 110. For example, by aggregating the Raman spectroscopy data collected throughout numerous perfusion processes on respective brains, a machine learning model can be trained to identify Raman spectroscopy data corresponding to particular responses by a human brain to a particular compound. Once the machine learning model has been trained, the Raman spectroscopy data generated by the sensor probe positioned on the surface of the brain 110 and/or spectroscopy system 107 following introduction of a pharmaceutical compound into the perfusate, along with other sensor data, can be provided to the trained machine learning model to predict certain responses or reactions relating to the administration of the pharmaceutical compound, such as a metabolic pathway of the pharmaceutical compound in the brain, an activation or deactivation of certain gene pathways in the brain, and/or physiological changes to the brain (e.g., swelling, tissue damage, infection, etc.). For example, the Raman spectroscopy data generated by the sensor probe positioned on the surface of the brain 110 and/or spectroscopy system 107 indicating the changes in chemical bonds within the brain can be provided to a trained machine learning model, which can utilize the Raman spectroscopy data to determine the likelihood that the brain is currently experiencing swelling or will experience swelling during the perfusion procedure.

While the spectroscopy system 107 and the spectroscopy probe positioned on the surface of the brain 110 of the perfusion system 100 have been described as being configured to perform Raman spectroscopy, it should be understood that such spectroscopy can include surface-enhance Raman spectroscopy (SERS). In addition, while the spectroscopy system 107 and the spectroscopy probe positioned on the surface of the brain 110 of the perfusion system 100 have been described as being configured to perform Raman spectroscopy, in some implementations, the spectroscopy system 107 and spectroscopy probe positioned on the surface of the brain 110 can be configured to other types of spectroscopy. For example, one or both of the spectroscopy system 107 and the spectroscopy probe positioned on the surface of the brain 110 can be configured to perform fluorescence spectroscopy, UV spectroscopy, or infrared spectroscopy.

Further, as an alternative to or in additional to using spectroscopy of the type described above, the perfusion systems described herein can use transcriptomics (including but not limited to qPCR, RNAseq, scRNAseq, snRNAseq, RNAscope, etc.), proteomics, mass spectrometry, ELISA, histology, genotyping, etc. to analyze the perfusion fluid and/or brain tissue samples. In some implementations, robust omics data (e.g., histology, immunofluorescence, metabolomics, bulk RNA sequence, single nucleus RNA sequence, and/or proteomics) are obtained across perfusion of 500 or more brains and utilized as training data for training the machine learning model. The omics data captured for each perfused brain can provide an indication of brain health that can serve as a benchmark to determine performance of the perfusion process.

While the syringe pump manifolds 167, 1167 and the dialysis filtration system 109 have been described as being controlled in order to provide concentrations of a drug according to a predetermined release profile, in some implementations, the uptake and metabolism of a particular drug is tracked in real time using the Raman spectroscopy system 107 and/or the Raman spectroscopy probe 172, as described above, and the syringe pump manifold 167 and dialysis filtration system 109 are dynamically controlled to increase or decrease the concentration of the drug in the perfusate based on the brain's uptake and metabolism of the drug, as detected using the Raman spectroscopy system 107 and/or the Raman spectroscopy probe 172. By dynamically adjusting the concentration of the drug in the perfusate using the syringe pump manifold 167 and the dialysis filtration system 109 in response to real-time Raman spectroscopy data indicating the uptake and metabolism of the drug by the brain, an optimized release profile for the drug that provides increased uptake and optimized metabolism of the drug by the brain can be determined.

While the sensor module 2107 has been described as being fluidly coupled downstream of the in-line Raman spectroscopy system 107, in some implementations, one or more sensors 1116, 1117, 1118, 1119 of the sensor module 2107 are alternatively or additional located at other positions along the venous circuit 108. For example, in some implementations, one or more sensors 1116, 1117, 1118, 1119 of the sensor module 2107 are positioned along the sensor line 11124, and the sensor module 2107 can be used to detect the presence, concentration, and level of oxidation of an oxygen carrier protein (e.g., LtEc) within the perfusate.

In addition, referring to FIG. 3, while the cellular and metabolic activity of the brain have been described as being determined based on performing Raman spectroscopy using the Raman spectroscopy system and/or the Raman spectroscopy probe 172, in some implementations, tissue samples 406 of the brain 110 and/or samples of the perfusate exiting the brain 110 are alternatively or additionally collected during and/or after perfusion of the brain 110 by the system 100, and one or more tests or assays are performed on the collected brain tissue samples 406 and/or collected perfusate samples to determine cellular and metabolic activity of the brain 110 during the perfusion procedures. Various testing procedures can be performed on the collected brain tissue samples 406 and the collected perfusate samples including, but limited to, spectroscopy, gene sequencing, cytokine analysis, anti-body analysis, genomic analysis, transcriptomic analysis, proteomic analysis, metabolomic analysis. In some implementations, complete omic analysis can be performed on one or more of the collected brain tissue samples 406 and the collected perfusate samples 402, 404.

In addition, the systems and methods described herein can be used to perform validation of biomarkers produced by the brain in response to introduction of one or more therapeutic agents. In some implementations, the omic analysis performed on one or more of the collected brain tissue samples 406 and the collected perfusate samples 402, 404 can be used to validate physiological activity of reactive oxygen species (ROS) and glycolytic stress pathways across brain cell types.

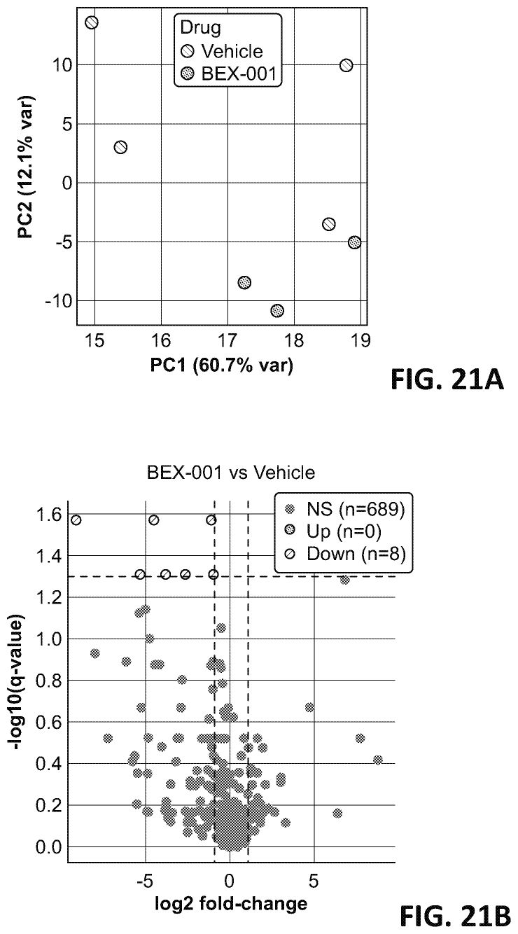

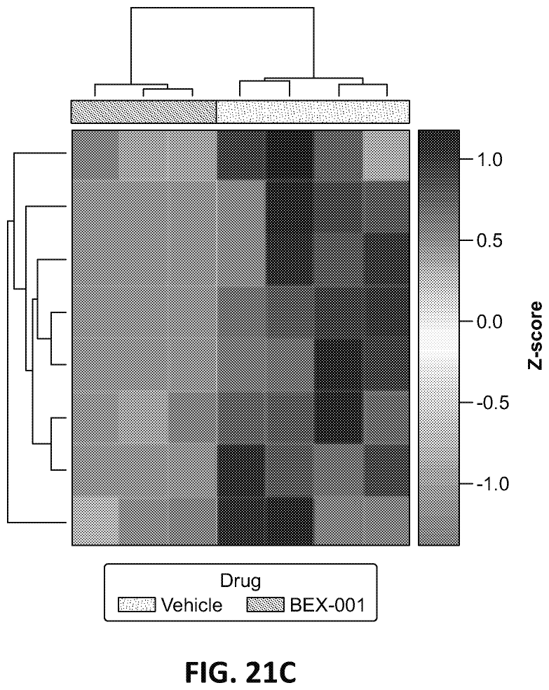

For example, referring to FIGS. 21A-21C, perfusate samples (e.g., venous effluent) were collected from drug-treated (BEX-001)-perfused brains and vehicle-perfused brains after 24 hours of perfusion and were subjected to untargeted metabolomics to profile all detectable metabolites in the samples. Despite the natural variation across individual brains, 8 metabolites showed significant differences between BEX-001 and vehicle treatment. Specifically, the principal component analysis (PCA) depicted in FIG. 21A revealed variation across individual brains on the first principal component (PC) and response to drug on the second PC. In general, PCA analysis begins from the interrelationship between the original variables, linearly transforms them to several independent composite indicators (i.e., principal components) according to the principle of variance maximization, takes two to three principal components for graphing, visually describes the differences in metabolic patterns and clustering results between different groups, and finally searches for the original variables that contribute to intergroup classification as biomarkers through loadings plots. See, e.g., Chen Y et al., Metabolites. 2022 Apr. 15; 12(4):357. FIG. 21B shows a differential analysis (corrected with Welch's t-test with Benjamini-Hochberg for false discovery) which showed that 8 metabolites were significantly reduced with BEX-001 treatment. FIG. 21C shows a heatmap of metabolite abundances across the treatment groups and timepoints for significant metabolites identified in the volcano plot of FIG. 21B. The rows of the heatmap represent metabolites and the columns of the heatmap represent the samples. The data showed that one of these metabolites fell within the expected pathway, and was independently identified in a different model system, demonstrating that real metabolomic changes can be identified with a small sample size (e.g., n≤3 group size) using the perfusate systems disclosed herein.

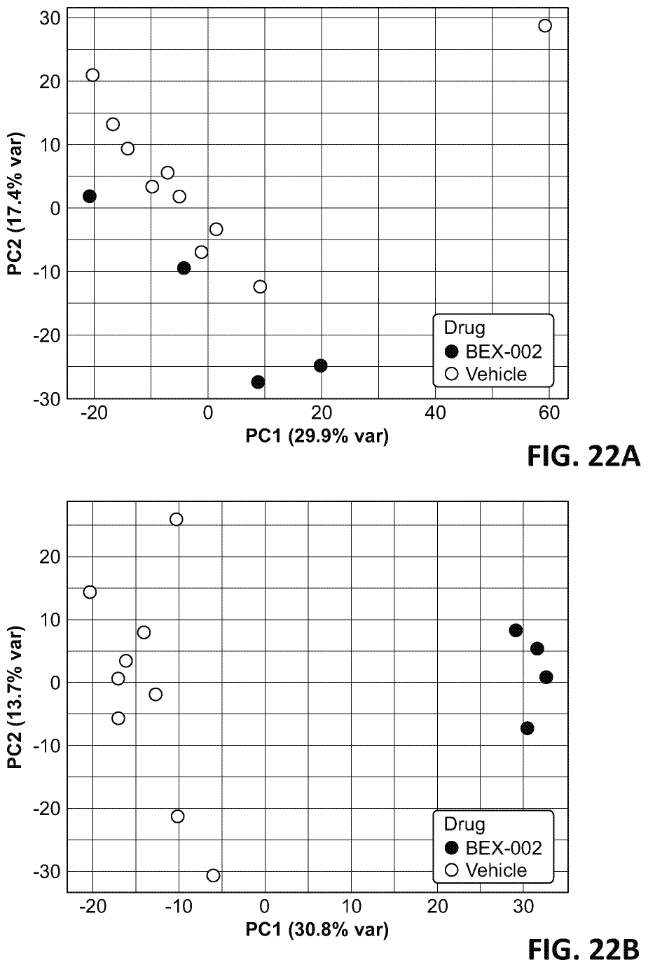

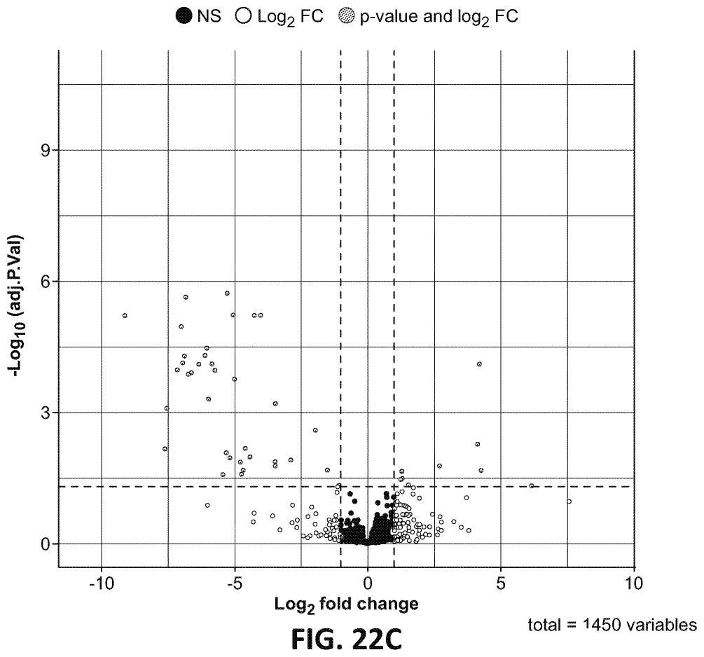

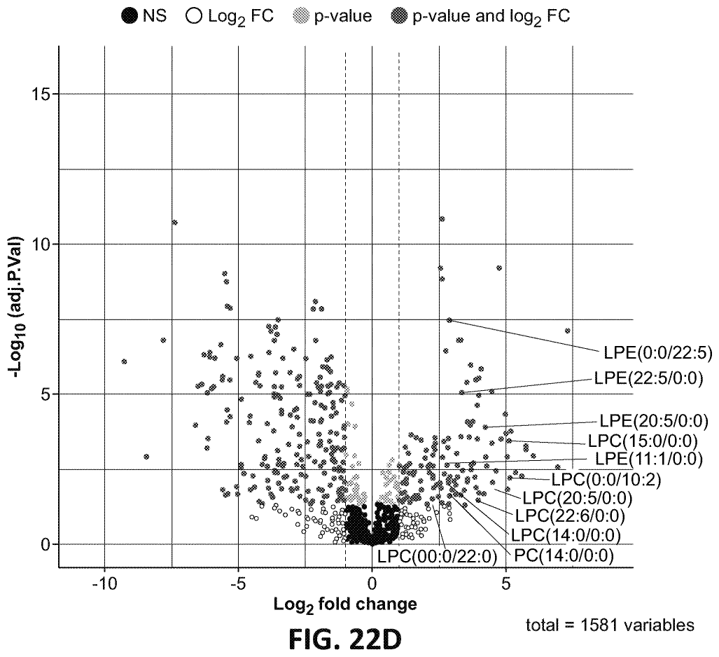

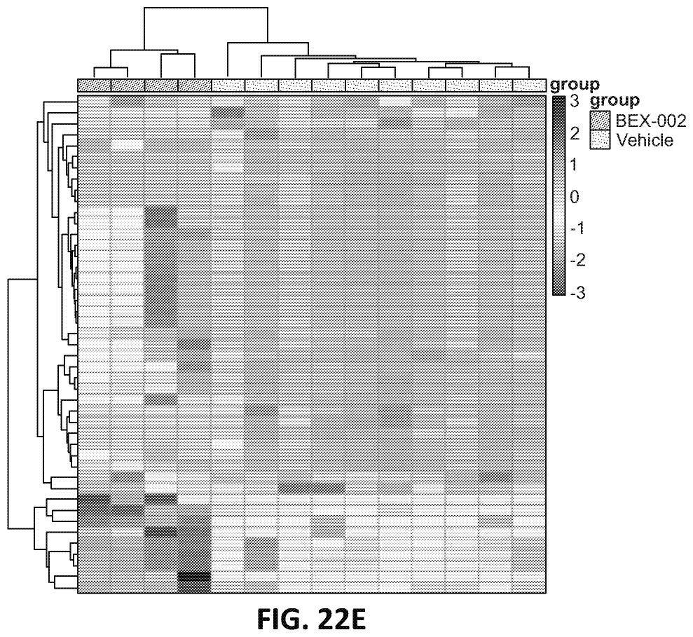

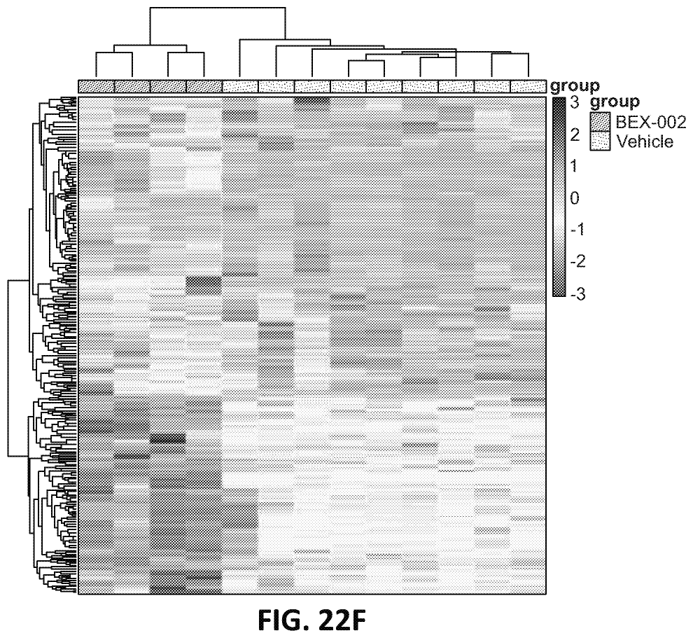

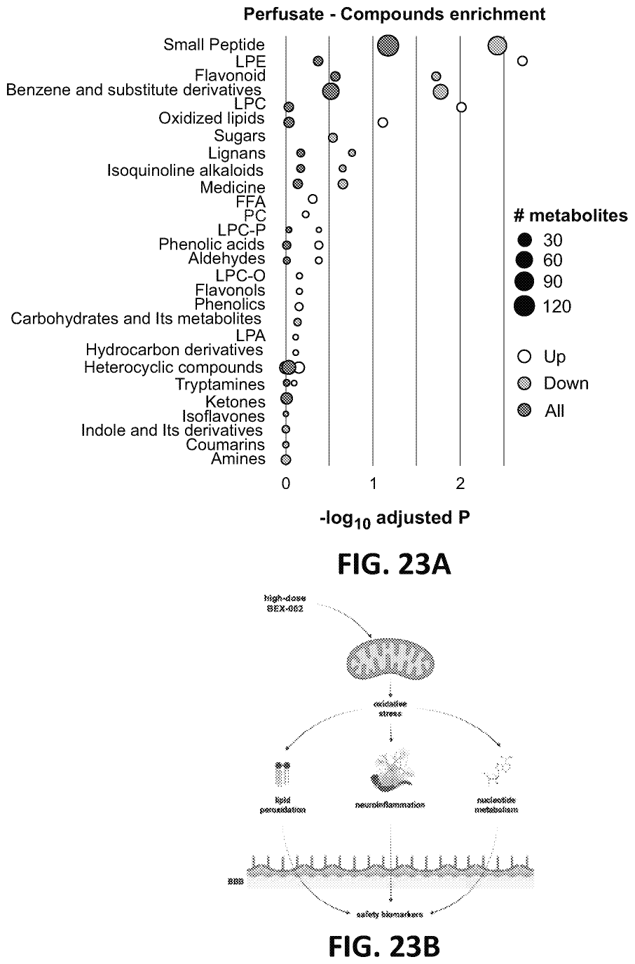

Further, referring to FIGS. 22A-22F and FIGS. 23A and 23B, prefrontal cortex (PFC) tissue samples and perfusate samples were collected from perfused brains that were treated with a drug (BEX-002) (“drug-treat brains”) and perfused brains that were not treated with the BEX-002 drug (“vehicle-perfused brains”) after 24 hours of perfusion. The samples were then subjected to untargeted metabolomics to identify metabolic biomarkers to be used as a safety signal for a high drug dose (e.g., a biomarker of drug toxicity). The principal component analysis (PCA) shown in FIG. 22A demonstrated that drug-treated PFC samples largely overlapped with vehicle. By contrast, the PCA shown in FIG. 22B revealed that drug-treated perfusate samples separated dramatically from vehicle. Differential expression analysis of bulk metabolomics data was performed using limma according to methods known in the art. See, e.g., Smyth, G. K. (2005). limma: Linear Models for Microarray Data. In: Gentleman, R., Carey, V. J., Huber, W., Irizarry, R. A., Dudoit, S. (eds) BIOINFORMATICS AND COMPUTATIONAL BIOLOGY SOLUTIONS USING R AND BIOCONDUCTOR. Statistics for Biology and Health. Springer, New York, NY. Differential expression analysis revealed 45 metabolites with statistically significant changes in the PFC (FIGS. 22C and 22E) compared to 388 metabolites in the perfusate (FIGS. 22D and 22F). In this example, the untargeted metabolomics revealed modest changes in the prefrontal cortex after 24 hours of perfusion comparing BEX-002 treatment (n=4 brains) with vehicle treatment (FIGS. 22A, 22C, and 22E). However, by taking advantage of the ability of the perfusate to capture the cumulative molecular output of the brain, a much greater effect was observed in the metabolomic profile of the perfusate comparing drug (BEX-002) to vehicle (FIGS. 22B, 22D, and 22F). As shown in FIG. 23A, a pathway analysis of metabolite changes observed in the perfusate samples (i.e., the samples depicted in FIGS. 22B, 22D and 22F) in response to high-dose BEX-002 demonstrated a change in oxidized lipids, inflammatory mediators, and nucleotides—such as lysophosphatidylethanolamine (LPE), lysophosphatidylcholine (LPC), lysophosphatidic acid (LPA), free fatty acid (FFA), and PC, phosphatidylcholine (PC). Pathway analysis suggested that the mechanism for BEX-002 toxicity was impairment of mitochondrial function, leading to oxidative stress and neuroinflammation that was detected by metabolite changes in peripheral biofluids (FIG. 23B).

In one experiment, deep multiomic profiling (RNAseq, quantitative proteomics, and untargeted metabolomics) was performed on brains following 24 hours of perfusion and the data collected for the perfused brains was compared with in vivo controls. Based on the comparison, molecular signatures of the perfused brains generally overlapped closely with the in vivo controls, but gene expression differences indicated acute microglial activation and oligodendrocyte vulnerability in the perfused brains. As a result, it was determined that the incorporation of anti-inflammatory cytokines for microglial response and key growth factors for oligodendrocyte into the perfusate could improve the overall health of a brain and extend the length of perfusion.

While the perfusion system 100 has been described as being used to determine the uptake and efficacy of a pharmaceutical compound, the perfusion system 100 can similarly be used to determine the uptake and efficacy of any of various other therapeutic agents, including, but not limited to, antibodies, biologics, viral vectors, lipid nanoparticles, peptides, etc.

In some examples, a particular gene therapy for treating a particular disease can be tested using the perfusion system 100. For example, a single vector or a collection of vectors carrying respective DNA segments can be added to the perfusate in the perfusate reservoir using similar procedures described herein for introducing a pharmaceutical compound to the perfusate, and the brain 110 can be perfused with the perfusate containing the vector(s). The used perfusate exiting the brain and the brain tissue can be tested using techniques described herein to determine whether the vector(s) have crossed the blood-brain barrier of the brain 110, to determine the vector transcytosis rates through the blood-brain barrier, and to determine an optimized release profile for the vector(s) to be most effectively taken up by the brain 110.

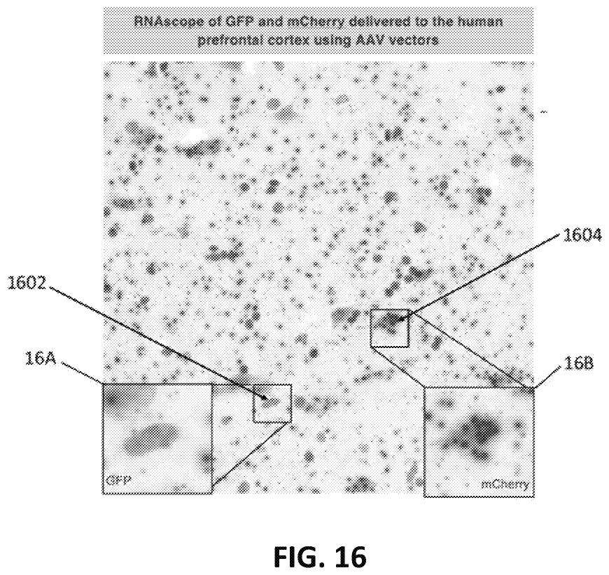

FIG. 16 is an RNAScope image showing that GFP and mCherry were successfully delivered to the frontal cortex of the brain using viral vectors, namely adeno-associated virus (AAV). Box 16A in FIG. 16 is an enlarged view of region 1602 showing evidence of GFP, while box 16B is an enlarged view of region 1604 showing evidence of mCherry. In this example, rather than introducing a chemical compound into the perfusate of the perfusion system 100, the viral vectors carrying the GFP and mCherry are introduced into the perfusate. The presence of the GFP and mCherry in the frontal cortex indicates that the viral vectors used were able to cross the blood-brain barrier and reach the frontal cortex of the brain.

Introduction of viral vectors can also be used to confirm that the perfused brain is capable of performing certain biological processes, such as maintaining the blood-brain barrier. In one experiment, in order to test whether the blood-brain barrier of a brain subjected to perfusion remained intact, a brain-targeted, clinically approved viral vector (AAV9) and a clinically approved viral vector that does not cross the blood-brain barrier (AAV2) were both delivered to the brain systemically by introducing the AAV9 and AAV2 viral vectors into the perfusate. FIG. 19 is an RNAScope generated following a 24 hour perfusion in which AAV9 and AAV2 were introduced into the perfusate. As indicated by the expression of the respective AAV cargo at the mRNA level, as shown in FIG. 19, AAV9 crossed the blood-brain barrier in the perfused human brain after 24 hours of systemic delivery, while AAV 2 did not cross the blood-brain barrier following 24 hours of perfusion. For example, region 1920 of FIG. 19 shows evidence of AAV9 present within the brain parenchyma without showing evidence of AAV2 in the brain parenchyma. Rather, as can be seen in region 1922 of FIG. 19, the AAV2 showed transduction primarily in vasculature of the brain. The functional effect of the viral vectors can be determined by measuring their ability to (1) cross the blood-brain barrier, (2) transduce brain cells, (3) express the cargo mRNA, and (4) translate the mRNA into protein.

Integrity of the blood-brain barrier of a brain following a two week perfusion can be determined based on measuring protein expression and the downstream biological effect of the introduction of viral vectors (in addition to mRNA expression of the AAV cargo). In particular, following a two week perfusion, the blood-brain barrier can be confirmed as being intact based on demonstrating mRNA and protein expression of the AAV9 cargo in neurons of the perfused brain combined with minimal expression of the AAV2 cargo in the perfused brain.

While methods of using the perfusion system 100 to test whether therapeutic agents can cross the blood-brain barrier, in some cases, the perfusion system 100 is alternatively or additionally used to determine whether the blood-brain barrier of a brain is intact. In such cases, for example, one or more agents known to be incapable of crossing the blood-brain barrier (e.g., certain large and/or water soluble molecules) can be introduced into the perfusate and then any of the various testing techniques described herein can be used to determine whether the agent or agents has/have crossed into the brain. The presence of any of those agents in the brain would be evidence that the blood-brain barrier of the brain is no longer intact. This may help researchers to determine whether test data related other therapeutic agents tested on that brain (particularly test data showing whether those therapeutic agents were able to cross the blood-brain barrier) are reliable. In some implementations, the integrity of the blood-brain barrier is tested by introducing Evans-blue into the perfusate and determining that 5% or less of the Evans-blue has leaked from the brain compared to a baseline amount.

The function of the blood-brain barrier of a brain 110 perfused using the system 100 can be tested by perfusing the brain 110 with related antihistamine drugs (H1 blockers) that do or do not cross the blood-brain barrier, which is supported by a plethora of human data, and measuring the penetrance of each of the perfused drugs into the brain tissue. In an example experiment, a brain 110 can be perfused with fexofenadine, cetirizine, and diphenhydramine. Fexofenadine is peripherally restricted, as demonstrated by receptor occupancy studies in humans using a PET ligand and based on the observation that fexofenadine does not cause any sedation in people. Fexofenadine has minimal brain penetrance and does not achieve appreciable brain exposures because of poor permeability of fexofenadine into brain tissue. Cetrizine is a moderately brain penetrant antihistamine and is a P-glycoprotein (P-gp) substrate. Diphenhydramine has well-established blood-brain barrier permeability and high brain penetrance based on PET studies and its established sedating effects.

All three of the antihistamine drugs (fexofenadine, cetirizine, and diphenhydramine) can be administered to the brain 110 through the perfusate and the pharmacokinetics of the drugs can be characterized by measuring concentrations of each drug in the perfusate over time using liquid chromatography-mass spectrometry (LC-MS). In addition, the concentration of each drug can be measured in various brain regions (Kp) at the end of the 24-hour perfusion after flushing the brain vasculature with phosphate-buffered saline to remove residual drug in vessels. The free fraction of each of the three perfused antihistamine drugs can be measured based on performing microdialysis (e.g., using microdialysis system 1502) to determine concentrations of each of the three perfused antihistamine drugs in the interstitial fluid of the cortex of the perfused brain 110 and in the cerebral spinal fluid produced in the cerebral ventricles by the choroid plexus of the perfused brain 110 during perfusion. The unbound brain-to-perfusate ratio (Kp, uu) for each antihistamine drug can be determined and the blood-brain barrier of the perfused brain 110 can be confirmed as being intact based on the brain-to-plasma ratio for all of the antihistamine drugs being calculated to within two-fold of reported human values for each respective drug. In addition, in order to confirm drug exposure in the brain for each antihistamine drug, ex vivo receptor occupancy studies can be conducted on brain slices using radioligands, and the blood brain barrier of the perfused brain 110 can be considered to be physiologically intact if brain-to-plasma ratios are calculated to within two-fold of reported human values.

The measured concentrations of the drugs in the perfusate and in the brain tissue, the free fraction of the drug in the interstitial fluid of the cortex and in the cerebral spinal fluid, and the drug exposure in the brain can be analyzed and compared to human brain penetrance data for each respective antihistamine drug to determine whether the blood-brain barrier of the perfused brain is intact and functioning normally following perfusion (e.g., following a 24-hour perfusion period). For example, evidence of P-gp-mediated transport across the blood-brain barrier of the perfused brain 110 can be identified based on detecting reduced brain penetration of cetirizine relative to diphenhydramine. Further, the blood-brain barrier of the perfused brain can be identified as being intact based on detecting a higher concentration of diphenhydramine than cetirizine in the brain tissue and a higher concentration of cetirizine than fexofenadine in the brain tissue. Further, concordance between LC-MS measurements, microdialysis measurements (cortex interstitial fluid and cerebral spinal fluid), and receptor occupancy measurements can also indicate that the blood-brain barrier is intact and functioning properly. Further, target engagement of each of the antihistamine drugs within the brain tissue can be confirmed based on determining that the ex-vivo receptor occupancy in the brain slices is consistent with the measured drug exposure levels and that there is a correlation between the receptor occupancy and unbound brain concentrations.

In some implementations, the predictive value of the perfusion system 100 can be tested by performing blinded mock trials using drugs that have already been performed in human clinical trials. For example, the predictive value of the perfusion system 100 can be determined based determining that the measurements following perfusion correlate with known clinical outcomes (e.g., efficacy vs. failure of a particular drug) and that the post-perfusion measurements differentiate drugs with clinical benefit versus mechanistic but non-efficacious or unsafe profiles.

In one example mock trial, brains are altered to induce ischemia unilaterally by medial cerebral artery occlusion and the perfusion system 100 is used to deliver nimodipine to a portion of the altered brains and to deliver clazosentan to the other portion of the altered brains. Nimodipine is an L-type Ca2+ channel blocker that prevents calcium influx into cerebral arterial smooth muscle cells, leading to vasodilation. Nimodipine is approved for patients with subarachnoid hemorrhage (SAH) caused by a ruptured intracranial aneurysm to prevent delayed ischemic brain injury. Conversely, clazosentan is an endothelin-A receptor antagonist and in clinical trials, clazosentan improved vasospasm, but did not reliably improve functional outcomes. Following perfusion, the pharmacodynamic effects of both drugs is determined by measuring cerebral artery flow rates. Further, as regional infarct injury burden, as measured based on histology, blood-brain barrier permeability, edema, oxidative injury markers, and microvascular flow rate of each of the brains are measured as primary endpoints.. For example, based on the measurements, the predictive value of the perfusion system 100 can be determined based on determining that nimodipine demonstrated reduction in injury metrics in the brains perfused with nimodipine are consistent with clinical benefits of nimodipine. The predictive value of the perfusion system 100 can be further confirmed based on the measurements for brains perfused with clazosentan showing discordance between vasospasm improvement and injury/functional surrogates.

In addition, following perfusion with either nimodipine or clazosentan using the perfusion system 100, protein levels of Glial fibrillary acidic protein (GFAP), Ubiquitin C-terminal hydrolase L1 (UCH-L1), and Neurofilament light chain (NfL) in the perfusate and metabolic recovery are measured as secondary endpoints. The predictive value of the perfusion system 100 can be determined based on detecting that the measured biomarker trends correlate with extent of injury and treatment response and that differentiation exists between treatment arms based on biomarker profiles.

The perfusion system 100 can also be used to test drugs that failed in clinical trials. In one example experiment, the perfusion system 100 was used to perfuse brains 110 with avagacestat, which is a γ-secretase inhibitor that successfully completed Phase I and Phase II clinical trials in healthy volunteers and Alzheimer's disease patients, respectively, and impacted the targeted pharmacodynamic biomarker, namely the reduction of amyloid beta (Aβ) measured in the cerebral spinal fluid. However, avagacestat ultimately failed in clinical trials for safety reasons due to patients suffering from worsening cognition and increased incidence of skin cancer. In the example experiment, the perfusion system 100 perfuses brains 110 with avagacestat using doses and dynamic pharmacokinetic profiles that match published drug exposures measured in the clinical trials. During the experiment, the Aβ levels in the brain tissue, the cerebral spinal fluid, and perfusate are measured and proteomics to determine the effects of avagacestat on other γ-secretase substrates, such as Notch pathway components. Based on the experiment, it can be determined whether avagacestat changes Aβ levels in the cerebral spinal fluid to the same extent as observed in clinical trials, and whether other γ-secretase substrates were impacted at the same concentrations.

In some implementations, the perfusion system 100 can be used to test the efficacy of combinations of multiple novel therapies (e.g., before entering clinical trials for the therapies). Successful treatment of certain diseases, such as Alzheimer's disease, may require a combination of therapies impacting complementary pathways when individual drugs do not demonstrate efficacy alone. However, testing combinations of novel drugs in clinical trials is complex and often not practical, since each drug would need to be tested in a dose response at each dose of the other drug or drugs to determine safety and efficacy prior to testing the combination. The perfusion system 100 can be used to perform efficacy testing of combinations of multiple novel therapies (e.g., novel drugs).

In one example experiment, the perfusion system 100 is used to combine mechanisms that target metabolic dysfunction, neuronal stress pathways, and neuroinflammation by perfusing each brain with semaglutide, blarcamesine, and the P2X7 antagonist JNJ-54175446 simultaneously. Semaglutide is a GLP-1 receptor agonist that targets metabolic dysfunction, and it is hypothesized that combining semaglutide with other mechanisms would result in synergistic disease-modifying efficacy. Blarcamesine is a sigma-1 receptor agonist that targets neuronal stress pathways, appears to support several biological processes that are perturbed in Alzheimer's disease, including synaptic function, mitochondrial metabolism, and neuronal stress resistance, and has demonstrated evidence of slowing cognitive decline in clinical trials. P2X7 antagonists target neuroinflammation and have shown positive results in animal models of Alzheimer's disease, but have only been tested in the clinical trial for other indications, including Major Depressive Disorder.

In the example experiment, each drug will be tested alone using the perfusion system 100 in three doses predicted to span the efficacious range (n=6 per dose per drug, 54 runs total). The efficacy of each drug will be determined by measuring proximal pharmacodynamic effects and functional pharmacology of the perfused brains. For brains 110 perfused with semaglutide, efficacy of the drug is determined based on detecting glucose uptake measured in real-time, increased cAMP response element-binding protein (CREB) and Akt phosphorylation, increased Brain-Derived Neurotrophic Factor (BDNF), increased adenosine triphosphate (ATP), and reduced Interleukin-1 beta (IL-1β), tumor necrosis factor-alpha (TNF-α), and NLRP3 levels. For brains 110 perfused with blarcamesine, efficacy of the drug is determined based on detecting an increase in pCREB and BDNF, reduced neuroinflammation, and increased ATP. For brains 110 perfused with JNJ-54175446, efficacy of the drug is determined based on detecting a reduction in neuroinflammation, particularly IL-1β and IL-18, and a detection of NLRP3 pathway activation. Concentration of each of the drugs in the respective perfused brains is measured in the brain by LC-MS. Efficacy of each of the three drugs for treating Alzheimer's disease will be determined based on post-perfusion measurements indicating improved synaptic healthy, indicated by increased synaptophysin staining, increased synaptic protein expression, synaptic biofluid markers that correlate with cognition (YWHAG:NPTX2 ratio), and reduction of biofluid NfL levels, indicating improved neuronal survival. Safety of each of the three drugs in the experiment will be determined based on histopathological staining indicating no significant tissue damage across doses and molecular markers of toxicity remaining within predefined acceptable thresholds.

Once the efficacy and safety of each of the three drugs (semaglutide, blarcamesine, and JNJ-54175446) has been determined based on the initial perfusion procedures, the perfusion system 100 can be used to test and identify the optimal combination of the drugs for treating Alzheimer's disease. Perfusion procedures are performed using the perfusion system 100 to test all pairwise combinations of the drugs and all three combined (n=6 per group, 24 runs total). The dosages of each of the drugs in the combinations can be determined based on a efficacy versus safety balance determined based on the measurements generated in the single-agent perfusion runs. Post-perfusion measurements will be performed to test the pharmacodynamic, efficacy and safety endpoints described above for each combination of the drugs in order to identify synergy between the drugs. Drug combinations with synergy demonstrate greater than additive effects of the two individual drugs. For example, efficacy of the various combinations of the drugs can be determined based on determining whether any combinations of the drugs improves synaptic and neuroinflammatory metrics compared to perfusion using one of the drugs individually. Safety of the various combinations of drugs can be determined based on histopathological staining indicating no significant tissue damage across doses and molecular markers of toxicity remaining within predefined acceptable thresholds Further, safety of the various combinations of drugs can be determined based on determining that the combinations of drugs did not result in toxicity greater than the toxicity profiles for the individual drug perfusion procedures. Based on the perfusion testing of the combination of drugs using the perfusion system 100, one or more combination therapies can be recommended for clinical trials.

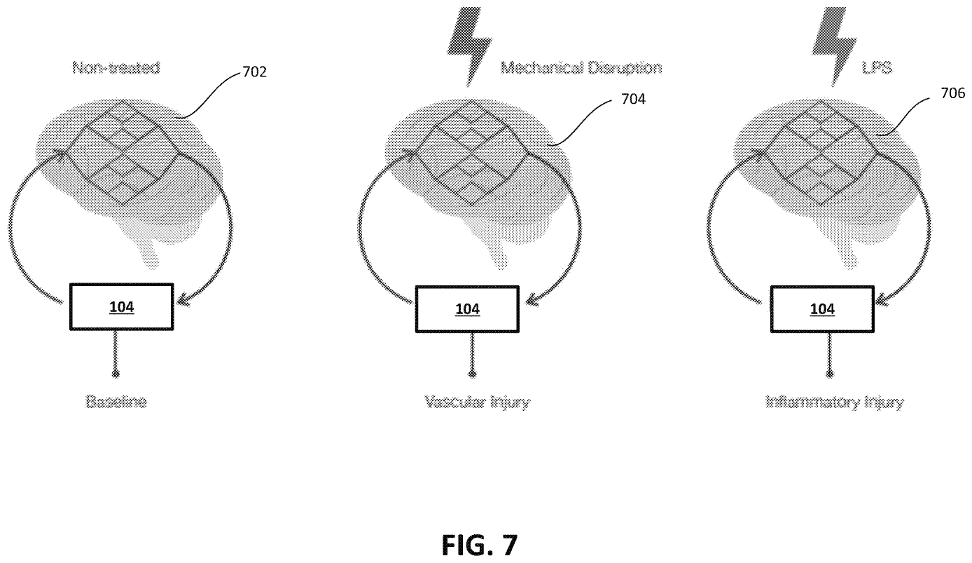

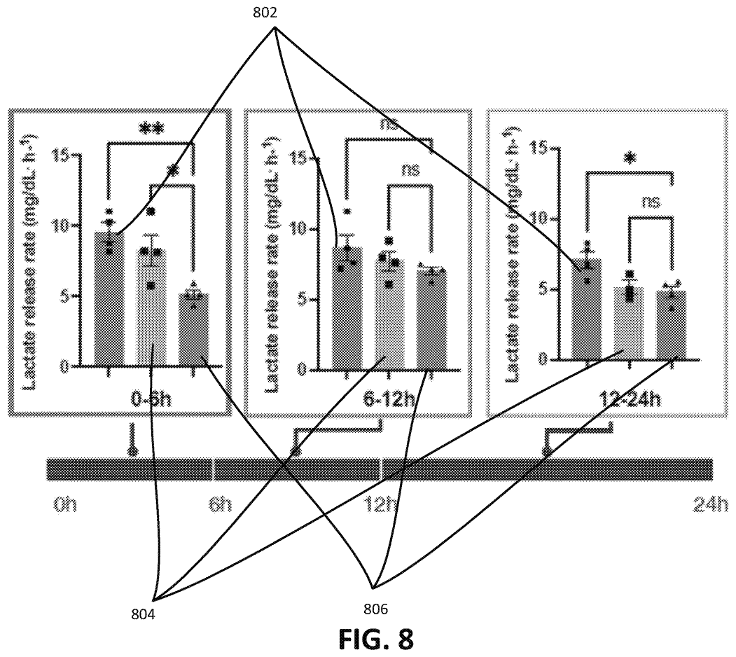

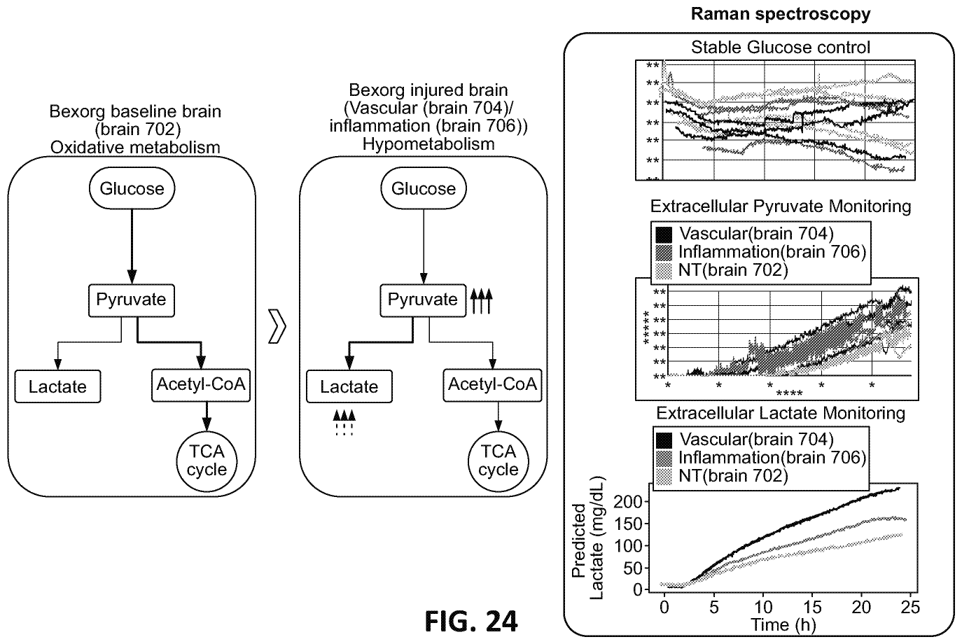

In addition, while the perfusion system 100 has been described as being used to perfuse and test brains that have not otherwise been mechanically or chemically altered, the perfusion system 100 can be used to perfuse and tests brains that have been mechanically or chemically altered (e.g., to simulate vascular and inflammatory injuries) prior to perfusing the brain with the system 100. For example, referring to FIGS. 7 and 8, an experiment was conducted in which three brains 702, 704, 706 were each perfused using the perfusion system 100 and the rate of lactate released by each of the brains 702, 704, 706 was monitored throughout the perfusion process (e.g., using the Raman spectroscopy system 107 of FIG. 1). The first brain 702 (“baseline brain 702”) was not mechanically or chemically altered prior to being perfused by the perfusion system 100. The second brain 704 was mechanically modified to mimic vascular disruptions prior to perfusion of the second brain 704 using the perfusion system 100, effectively replicating the impact of vascular injury on brain tissue. The third brain 706 was treated with lipopolysaccharide (LPS) during perfusion using the perfusion system 100 to mimic pathophysiological conditions associated with vascular impairments and inflammation in the brain. The rate of lactate release by each of the three brains 702, 704, 706 was tested throughout the 24 hour perfusion process (e.g., by performing Raman spectroscopy on the used perfusate exiting the brains 702, 704, 706 using the Raman spectroscopy system 107). As can be seen in FIG. 8, the rate of lactate release 802 for the untreated brain 702 was higher than both the rate of lactate release 804 for the mechanically altered brain 704 and the rate of lactate release 806 for the brain 706 perfused with lipopolysaccharide (LPS). FIG. 24 shows real-time metabolite monitoring for the three brains 702, 704, 706. Specifically, glucose, pyruvate, and lactate were monitored in real-time in the perfusate using the Raman spectroscopy system 107 of FIG. 1. Glucose levels were maintained at stable levels in all conditions using the homeostatic maintenance function of the perfusion system 100. However, pyruvate and lactate levels increased to a greater degree in injured brain compared to non-treated brains. These data indicated that there was a shift from aerobic respiration through the TCA cycle in brain 702 to glycolytic energy production in brains 704 and 706. By utilizing the perfusion system to perfuse and test brains that are mechanically or chemically altered to simulate vascular and inflammatory injuries, the perfusion system 100 can be used to effectively study mechanisms and potential treatments for brain injuries.

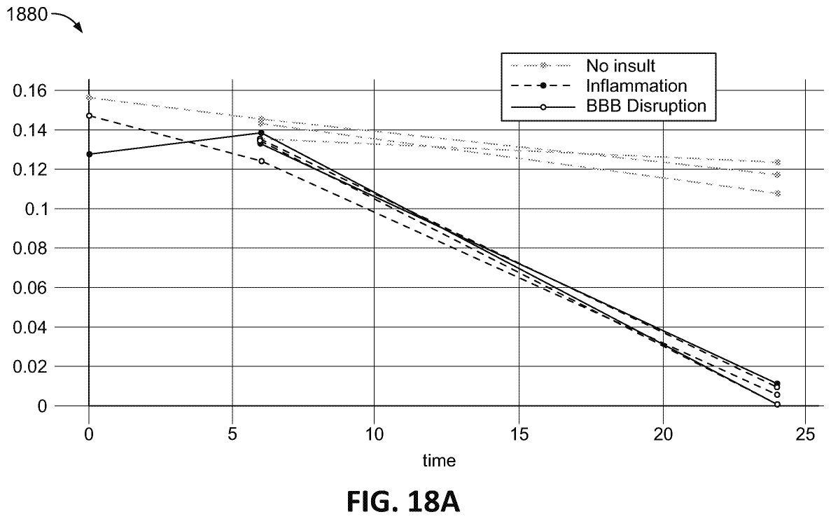

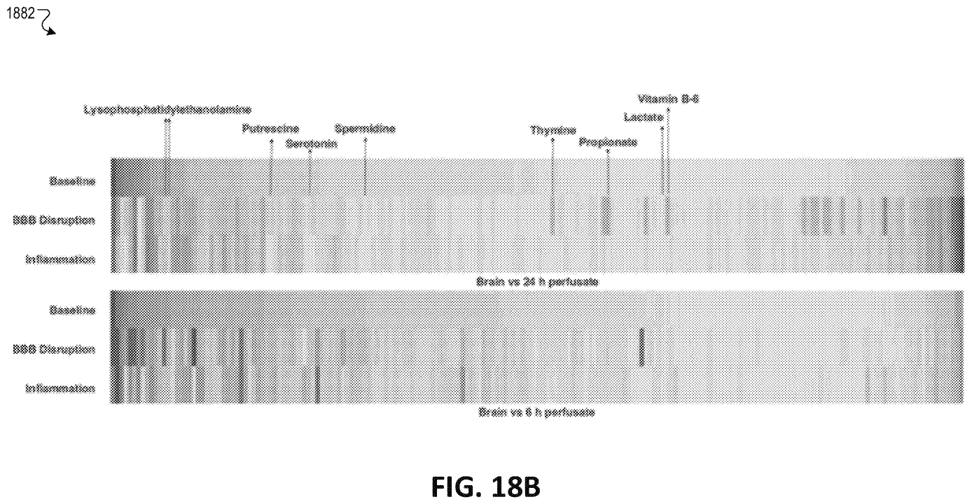

Further, referring to FIGS. 18A and 18B, an experiment was conducted in which nine brains were perfused using the perfusion systems described herein. Of the nine brains perfused during the experiment, three of the brains were unaltered, three of the brains were altered to mimic an inflamed brain, and three of the brains were mechanically altered to mimic a brain with disruption to the blood-brain barrier. During each perfusion process, the adenosine levels produced by the brains (as depicted in the graph 1880 in FIG. 18A) and the amount of vitamin B-6 leaked from the brain into the perfusate (as depicted in the heat map 1882 in FIG. 18B) was measured. As can be seen in FIGS. 18A and 18B, injury to the brain (e.g., due to inflammation or blood-brain barrier disruption) resulted in decreased adenosine levels and an increased amount of vitamin B-6 leakage out of the brain tissue into the perfusate. As a result, adenosine and vitamin B-6 were identified as potential biomarkers for identifying brain injury during perfusion. In addition, supplementation of adenosine and vitamin B-6 through the perfusate could be used to improve brain health during perfusion and extend the length of perfusion.

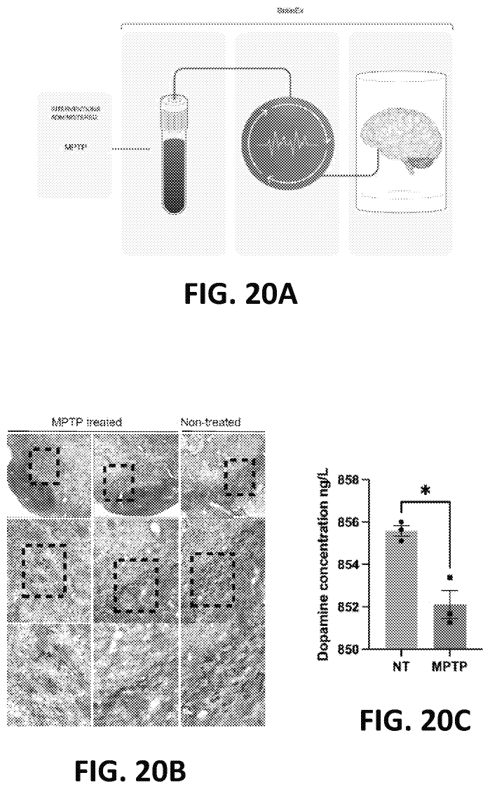

Additionally, referring to FIGS. 20A-20C, brains were chemically modified during perfusion thereby generating a Parkinsonian model system. As shown in FIG. 20A, a brain was treated with 1-methyl-4-phenyl-1,2,3,6-tetrahydropyridine (MPTP) during perfusion using the perfusion system 100. Tissue samples 406 were collected from the perfused brain 24 hours after MPTP perfusion treatment. As depicted in FIG. 20B, hematoxylin and eosin (H&E) staining of the tissue samples 406 demonstrated loss of dopaminergic (DA) neurons in the MPTP treated brain compared to an untreated brain. As depicted in FIG. 20C, plasma dopamine concentration was also reduced in the MPTP treated brain compared to plasma collected from an untreated brain (FIG. 20C).

While the perfusate system 100 has been described as being used to perfuse a human brain 110, the perfusion system 100 can be used to perfuse other mammalian brains, such as pig brains. In some implementations, the perfusate used in the perfusate system 100 may be altered in order to more effectively perfuse non-human mammalian brains. In addition, an alternate brain housing 102 may be used to better accommodate and support a pig brain. For example, in some implementations, when the perfusion system 100 is being used to perfuse a pig brain, the brain housing 102 is replaced with a pig brain housing that is smaller than the brain housing 102 used to house the human brain 110.

FIGS. 11A-11D depict an example brain housing 1102 that can be used to support a pig brain during a perfusion process performed using the system 100 of FIG. 1. As can be seen in FIGS. 11A-11D, the brain housing 1102 includes a basin 1104 and lid 1106. The lid 1106 is configured to releasably couple to the basin 1104 and can be separated from the basin 1104 to allow a pig brain to be positioned inside the interior of the brain housing 1102.

When the brain housing 1102 is fluidly connected to the system 100 of FIG. 1, the housing outlet line 176 extends from the brain housing 1102 and fluidly couples the brain housing 1102 to the perfusate reservoir 104. Referring to FIGS. 11A, 11B, and 11D, the brain housing 1102 includes a coupling 1142 connected to the side of the basin 1104. The coupling 1142 fluidly connects the interior of the basin 1104 of the brain chamber 1102 to the housing outlet line 176. In some implementations, the coupling 1142 is a quick-turn tube coupling. In some implementations, the brain housing 1102 includes a second coupling 1144 that can be coupled to the outlet line 176 or to another outlet line in order to increase the rate of drainage of fluid out of the brain housing 1102 to the perfusate reservoir 104.

The brain housing 1102 also includes a fluid coupling 1146 that fluidly connects the shunt line 156 to the interior of the brain chamber 1102. In some implementations, the coupling 1146 is a quick-turn tube coupling.

The brain housing 1102 also includes a three arm knob 1108 releasably coupled to the lid 1106 of the brain housing 1102. The knob 1108 serves as an ergonomic handle for opening the lid 1106 of the brain housing 1102.

The brain housing 1102 includes a level sensor 180 to detect the level of fluid (e.g., used perfusate) inside the basin 1104 of the brain housing 1102, and the solenoid valve 178 along the housing outlet line 176 is controlled to open or close based on signals generated by the level sensor 180. As depicted in FIG. 11D, the level sensor 180 is coupled to the basin 1104 of the brain chamber 1102. In some implementation, the level sensor 180 is coupled to the basin 1104 at the same or similar height along the basin 1104 as the fluid coupling 1146 that fluidly connects the shunt line 156 to the interior of the brain chamber 1102. In some implementations, the level sensor 180 is an optical level sensor, such as an infrared level sensor. For example, the solenoid valve 178 is controlled to remain in a closed state and prevent flow of used perfusate from the brain housing 1102 into the perfusate reservoir 104 until the level sensor 180 generates a signal indicating that the fluid level inside the brain housing 1102 has exceeded a threshold fluid level. In response to the level sensor 180 generating a signal indicating that the fluid level inside the brain housing 1102 has exceeded a threshold fluid level, the solenoid valve 178 and the solenoid valve 1121 along the venous sensor line 1129 are opened and the sensor pump 1122 is operated to pump used perfusate from the brain housing 1102 along the housing outlet line 176 and the sensor line 1124 to the perfusate reservoir 104. Once the level sensor 180 detects that the fluid level inside the brain housing 1102 has fallen below the threshold level, the solenoid valves 178, 1121 are closed, which prevents fluid from flowing from the brain housing 1102 to the perfusate reservoir 104 or the sensor line 1124.

The brain housing 1102 is configured to maintain the pig brain under normothermic conditions. For example, when the basin 1104 and lid 1106 of the brain housing 1102 are securely coupled together, a warm, humid environment is maintained within the brain housing 1102. In some implementations, the brain housing 1102 includes temperature and humidity controls configured maintain a particular temperature and humidity level to prevent drying or other injury to the brain.

Prior to placement of the pig brain in the brain housing 1102 for perfusion, the pig brain is fluidly coupled to a brain interface 1170 that is configured to fluidly couple the vasculature of the pig brain to the organ line 144 of the system 100. FIGS. 12A-12C depict an example brain interface 1170 for fluidly coupling a pig brain to the organ line 144. The brain interface 1170 includes a base plate 1172, two fluid input ports 1174, 1176, and two fluid output ports 1178, 1180.

In some implementations, the base plate 1172 is formed of a titanium alloy. In some implementations, the base plate 1172 is formed through laser sintering a titanium alloy. In some implementations, the base plate 1172 is formed through 3D printing. Referring to FIG. 12A, the base plate 1172 defines a plurality of drain holes 1182 therethrough that allow perfusate flowing out of the pig brain to flow through the base plate 1172 into the basin 1104 of the brain housing 1102 such that the perfusate flowing out of the pig brain is collected in the basin 1104.

Each of the fluid inlet ports 1174, 1176 of the brain interface 1170 are fluidly coupled to the organ line 144 using a y-connector and each inlet port 1174, 1176 of the brain interface 1170 receives fresh perfusate flowing out of the organ line 144 into the brain interface 1170. Referring to FIG. 12C, the brain interface 1170 defines flow paths 1184 fluidly coupling each of the fluid inlets ports 1174, 1176 of the brain interface 1170 to a respective outlet port 1178, 1180 of the brain interface 1170.

Prior to placing a pig brain in the brain housing 1102 and beginning perfusion of the pig brain, each of the carotid arteries of the pig brain is sutured to a respective fluid outlet port 1178, 1180 of the brain interface 1170. Once the carotid arteries of the pig brain are sutured to the fluid outlet ports 1178, 1180 of the brain interface 1170 and the inlet ports 1174, 1176 of the brain interface 1170 are fluidly coupled to the organ line 144, the pig brain and the brain interface 1170 can be positioned within the basin 1104 of the brain housing 1102. The lid 1106 of the brain housing 1102 is coupled to the basin 1104 to enclose the pig brain and brain interface 1170 within the brain housing 1102 throughout the perfusion process. Referring to FIG. 11B, the basin 1104 of the brain housing 1102 defines a slot 1110 that allows a portion of the organ line 144 to enter the basin 1104 with minimal air gaps when the lid 1106 is coupled to the basin 1104. In some implementations, the brain housing 1102 can be pressurized via the brain housing pressure line 1131 using the same methods as those described above to pressurize the brain housing 102.

As fresh perfusate flows along the organ line 144, the perfusate enters the fluid inlet ports 1174, 1176 of the brain interface 1170 and flows through the flow paths 1184 defined by the brain interface 1170, through the fluid outlet ports 1178, 1180 of the brain interface 1170, and into the carotid arteries of the pig brain coupled to the brain interface 1170. As previously discussed, the pressure and flow rate of the perfusate provided to the pig brain are controlled to enable the perfusate entering the carotid arteries of the brain to perfuse the entire vasculature of the brain, including the penetrating arterioles, pre-capillary arterioles, and capillaries of the brain.

While the brain interface 1170 has been depicted as having two fluid outputs, in some implementations, in order to perfuse a pig brain using the perfusion system 100, a brain interface having a single fluid output is provided, and a single artery of the pig brain is sutured to the single fluid output of the brain interface to fluidly couple the pig brain to the brain interface.

In addition, while the perfusion systems described herein have been described as being used to perfuse brains, in some implementations, the perfusion system can be used to perfuse other mammalian (e.g., human) organs. For example, the perfusion system can be used to perfuse kidneys, hearts, livers, lungs, pancreases, etc. In some implementations, an alternate interface is used to fluidly couple the non-brain mammalian organ to the perfusions system 100. Further, in some implementations, an alternate housing device is used to contain and collect perfusate expelled from the non-brain mammalian organ perfused by the system 100. In addition, the perfusate used in the perfusate system 100 may be altered in order to more effectively perfuse non-brain mammalian organs. For example, in some implementations, perfusate used to perfuse non-brain mammalian organs has a higher concentration of oncotically active particles compared to perfusate of brains in order to accommodate the higher capillary filtration rates that occur in non-brain tissue.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. An ex-vivo brain perfusion system for perfusion of a brain, the system comprising:
a reservoir configured to receive perfusate;
a housing configured to contain the brain;
a housing stand configured to automatically adjust a position of the brain housing;
an arterial circuit configured to fluidly couple the reservoir to the brain; and
a venous circuit fluidly coupling the housing to the reservoir.

2. The system of claim 1, the housing stand comprises:
a base;
a plurality of legs extending from the base; and
at least one linear actuator, wherein the at least one linear actuator is configured to extend and retract to automatically adjust the position of the brain housing.

3. The system of claim 2, wherein the at least one linear actuator is configured to extend and retract at predefined intervals throughout the perfusion of the brain.

4. The system of claim 3, wherein:
the system comprises at least one sensor configured to generate data indicate tissue viability of the brain; and
the at least one linear actuator is configured to extend and retract based on data received from the at least one sensor.

5. The system of claim 4, wherein the at least one sensor comprises at least one of a camera, a Raman spectrometer, a colorimeter, a fluorimeter, or a photometer.

6. An ex-vivo brain perfusion system for perfusion of a brain, the system comprising:
a reservoir configured to receive perfusate;
a housing configured to contain the brain;
an arterial circuit configured to fluidly couple the reservoir to the brain;
a venous circuit fluidly coupling the housing to the reservoir;
at least one sensor configured to generate data indicating one or more characteristics of the perfusate;
one or more processors configured to execute a machine learning model to process the data generated by the at least one sensor and determine one or more chemical components to be added to the perfusate to extend the length of perfusion; and
a syringe pump manifold fluidly coupled to the reservoir and configured to automatically introduce the one or more chemical components determined by the machine learning model.

7. The system of claim 6, wherein the at least one sensor comprises at least one of a Raman spectrometer, a colorimeter, a fluorimeter, or a photometer, a conductivity sensor, a pH sensor, a temperature sensor, or a viscosity sensor.

8. The system of claim 6, wherein the syringe pump manifold is configured to automatically introduce the one or more chemical components based on one or more command signals generated by the machine learning model.

9. The system of claim 6, wherein:
the system comprises a filtration system;
the machine learning model is configured to determine one or more chemical components to be removed to the perfusate to extend the length of perfusion; and
the filtration system is configured to be operated to remove the one or more chemical components from the perfusate in the reservoir.

10. The system of claim 9, wherein:
the filtration system comprises a dialyzer manifold comprising a plurality of dialyzers; and
the dialyzer manifold is configured to automatically divert flow of perfusate from a first dialyzer of the plurality of dialyzers to a second dialyzer of the plurality of dialyzers based on a sensor of the filtration system generating data indicating that the first dialyzer is clogged or malfunctioning.

11. The system of claim 10, wherein:
the filtration system comprises:
a dialysis solution receptacle configured to receive fluid flowing out of the dialyzer manifold; and
a pump configured to pump fluid from the dialysis solution receptacle to the reservoir;
the data generated by the at least one sensor indicates a ratio of small molecules to large molecules present in the perfusate in the reservoir; and
the pump is controlled to pump fluid from the dialysis solution receptacle to the reservoir in response to a determination that the ratio of small molecules to large molecules present in the perfusate in the reservoir is below a first threshold.

12. The system of claim 9, wherein:
the filtration system comprises a filter manifold comprising a plurality of mechanical filters; and
the filter manifold is configured to automatically divert flow of perfusate from a first mechanical filter of the plurality of mechanical filters to a second mechanical filter of the plurality of mechanical filter based on a sensor of the filtration system generating data indicating that the first mechanical filter is clogged or malfunctioning.

13. The system of claim 6, wherein:
the data generated by the at least one sensor is configured to be processed by the machine learning model to determine viability of tissue of the brain;
the machine learning model is configured to determine at least one chemical component to be added to the perfusate to improve viability of the tissue of the brain; and
the syringe pump manifold is configured to introduce the at least one chemical component identified by the machine learning model.

14. The system of claim 13, wherein:
the at least one sensor comprises a camera configured to take image data depicting the brain; and
the machine learning model is configured to perform machine learning based image processing on the image data to determine the viability of the tissue of the brain in real time.

15. The system of claim 6, comprising an impeller positioned within the reservoir and configured to mix the perfusate contained in the reservoir.

16. The system of claim 6, wherein the perfusion system comprises:
a supplemental reservoir configured to contain fresh perfusate; and
a supplement pump configured to pump fresh perfusate from the supplemental reservoir to the reservoir.

17. The system of claim 16, wherein the supplement pump is configured to pump fresh perfusate from the supplemental reservoir to the reservoir at predetermined intervals throughout perfusion of the brain. or the supplement pump is configured to pump fresh perfusate from the supplemental reservoir to the reservoir based on data generated by a viscosity sensor indicating that a viscosity of the perfusate in the reservoir is above a threshold viscosity.

18. The system of claim 6, comprising a microdialysis system fluidly coupled to the housing and configured to perform dialysis on interstitial fluid of the brain.

19. The system of claim 6, comprising:
a housing pressurization system comprising:
an air supply;
a fluid line fluidly coupling the air supply to the housing; and
a pressure regulator configured to control the pressure of air flowing from the air supply into the housing.

20. The system of claim 19, wherein:
the housing pressurization system comprises a pressure sensor configured to measure pressure inside the housing; and
the pressure regulator is an electronic pressure regulator configured to be controlled based on one or more signals generated by the pressure sensor.
